(19) United States
(12) Patent Application Publication
(10) Pub. No.: US 2024/0008994 A1
(43) Pub. Date: Jan. 11, 2024

(54) HUMERAL IMPLANT AND SYSTEMS AND METHODS FOR IMPLANTING THE SAME

(71) Applicant: HOWMEDICA OSTEONICS CORP., Mahwah, NJ (US)

(72) Inventors: Benjamin DASSONVILLE, Saint Hilaire du Touvet (FR); Gilles HENRY, Le Pont de Claix (FR); Vincent GABORIT, Saint Martin d'Hères (FR); Vincent COULANGE, Lyon (FR); George ATHWAL, London (CA); Pascal BOILEAU, Nice (FR); Christopher CHUINARD, Traverse City, MI (US); Philippe CLAVERT, Illkirch Graffenstaden (FR); Luc FAVARD, Montlouis (FR); James D. KELLY, II, San Francisco, CA (US); François SIRVEAUX, Villers Les Nancy (FR)

(73) Assignee: HOWMEDICA OSTEONICS CORP., Mahwah, NJ (US)

(21) Appl. No.: 18/369,138

(22) Filed: Sep. 15, 2023

Related U.S. Application Data
(63) Continuation-in-part of application No. PCT/US22/70304, filed on Jan. 24, 2022.
(60) Provisional application No. 63/200,608, filed on Mar. 17, 2021.

(51) Int. Cl. A61F 2/40; A61F 2/30; A61F 2/46

(52) U.S. Cl. CPC A61F 2/4059; A61F 2/30771; A61F 2/4612; A61F 2/30749; A61F 2/3094; A61F 2002/30784; A61F 2002/30827; A61F 2002/30433; A61F 2002/4658; A61F 2002/4681; A61F 2002/4631

(57) ABSTRACT

Various embodiments disclosed herein relate to stemmed and stemless humeral anchors, and implanting tools for use in shoulder arthroplasty procedures. For example, the humeral anchor can include a distal shaft portion, a proximal portion, and a metaphyseal portion connecting the distal shaft portion and the proximal portion. The distal shaft portion can include multiple apertures configured to receive a screw or one or more plugs. The proximal portion can include a stem face configured to be removably attached to an implanting tool or to couple to an anatomic insert or a reverse insert.

### CROSS-REFERENCE TO RELATED APPLICATION

This application is a continuation-in-part of International Application No. PCT/US2022/070304, filed on Jan. 24, 2022, which claims benefit to U.S. Provisional Application No. 63/200,608, filed Mar. 17, 2021, the disclosures of which are incorporated herein by reference in their entirety.

### BACKGROUND

### Field

The present application relates to reverse and anatomic shoulder prostheses for fracture repair.

### Description of the Related Art

Arthroplasty is the standard of care for the treatment of shoulder joint arthritis. A typical anatomical shoulder joint replacement attempts to mimic anatomic conditions. For example, a metallic humeral stem and a humeral head replacement are attached to the humerus of the arm and replace the humeral side of the arthritic shoulder joint. Such humeral head replacement can articulate with the native glenoid socket or with an opposing glenoid resurfacing device.

For more severe cases of shoulder arthritis, the standard treatment is a reverse reconstruction, which includes reversing the kinematics of the shoulder joint. A reverse shoulder prosthesis can be provided by securing a semi-spherical device (sometimes called a glenoid sphere) to the glenoid and implanting a humeral stem with a cavity capable of receiving the glenoid sphere.

As patient disease may progress after anatomic treatment, revision surgery may be necessary to perform a reverse reconstruction of the shoulder. In the known art, the change in the type of prosthesis is addressed either below the plane of resection or above the plane of resection. In prostheses that are converted from anatomic to reverse by a modularity below the plane of resection, removal of anatomic devices that have integrated into the patient's bony anatomy proves to be difficult for the surgeon, and could potentially cause excessive patient bone loss. One advantage of such conversion is that the reverse insert could partially reside below the resection plane and therefore reduce the distance between the cavity and the lateral contour of the humerus. Such position has proven to be beneficial to a reversed kinematics. In the contrary, in prostheses that are converted from anatomic to reversed above the plane of resection, using an adaptor, reverse kinematics are altered as the position of the cavity is further pushed out of the humerus by the addition of the adaptor above the resection plane. Such constructs are typically made of three components that present an extra modularity in comparison to two component constructs and could potentially cause disassembly or breakage of the construct. One possibility to limit the alteration of the kinematics and limit the modularity is to inverse the bearing surface material by having a harder cavity within the humerus and a softer semi-spherical device secured to the glenoid. But the proven clinical design and preferred embodiment is usually that the cavity is softer than the semi-spherical device.

In cases of displaced or dislocated 3- and 4-part proximal humeral fractures, the proximal humerus also needs to be reconstructed. Although hemi-arthroplasty procedures may be used for the treatment of such displaced fractures, the functional outcomes of these procedures are often reported as poor and unpredictable.

### SUMMARY

A convertible prosthesis that can be converted from an anatomic replacement to a reverse reconstruction without removal of parts integrated into the patient's bony anatomy is highly desirable. For improved patient outcomes, such a convertible prosthesis should respect the biomechanics of a true anatomic replacement while also performing well when converted into a reverse reconstruction. In some cases, it may also be desirable for the convertible prosthesis to be configured for use in a humeral fracture repair procedure.

Improved humeral anchors, components, assemblies, and methods are needed to provide more flexibility in working with soft tissue around the shoulder joint. Such anchors may benefit from multiple apertures that can each receive a plug or a screw for securing the humeral anchor in the patient. Such anchors may benefit from having a V-shaped profile to reduce the amount of bone the clinician has to remove from the patient to implant the humeral anchor.

In some aspects of the disclosure, a stem for a shoulder prosthesis is disclosed. The stem can include a medial side, a lateral side opposite the medial side, and a plurality of apertures. Each aperture of the plurality of apertures can be adapted to receive a screw or one or more plugs. The plurality of apertures can include a first aperture and a second aperture. The first aperture can be positioned proximal to the second aperture. Each of the first and second apertures can include a first opening on the medial side, a second opening on the lateral side, and a length measured along a longitudinal centerline therebetween. The longitudinal centerline of at least one of the first and second apertures can be angled relative to a longitudinal plane extending in a medial-lateral direction of the stem. Alternatively, the longitudinal centerline of each of at least one of the first and second apertures can be angled relative to a longitudinal plane extending in an anterior-proximal direction of the stem or relative to any plane of the stem.

The stem of the preceding paragraphs or as described further herein can also include one or more of the following features. Each of the first and second apertures can be angled in an anterior-posterior direction relative to the medial-lateral longitudinal plane. Alternatively, each of the first and second apertures can be angled in medial-lateral direction relative to the anterior-posterior longitudinal plane. The first and second apertures can be angled in opposite directions relative to the longitudinal plane. Alternatively, the first and second apertures can be angled in the same direction relative to the longitudinal plane. The stem can further include a distal shaft portion, a proximal portion, and a metaphyseal portion. The distal shaft portion can be adapted to be anchored in a medullary canal of a humerus. The proximal portion can having a stem face. The metaphyseal portion can extend between and connect the distal shaft portion and the proximal portion. The metaphyseal portion can include a medial portion and first and second lateral arms. Alternatively, the metaphyseal portion can include only one lateral arm or more than two lateral arms. The distal shaft portion can include the plurality of apertures. The distal shaft portion can include a plurality of grooves. The plurality of grooves can extend in a longitudinal direction. The plurality of grooves can be circumferentially spaced apart. Each of the plurality of grooves can narrow toward a distal tip of the stem. The first aperture can be positioned proximal to the plurality of grooves. The second aperture can extend through at least one of the plurality of grooves. Alternatively, the first and second apertures can both be positioned proximal to the plurality of grooves or extend through at least one of the plurality of grooves. The plurality of apertures can further include a third aperture positioned distal to the second aperture. The longitudinal centerline of each of the first and second apertures can be angled about 30° relative to the longitudinal plane of the stem. Alternatively, the longitudinal centerline of each of the first and second apertures can be angled at less than or more than about 30° relative to the longitudinal plane of the stem. For example, the angle can be about 15° or about 45°. The longitudinal centerline of each of the first and second apertures can be angled at different angles. For example, the longitudinal centerline of the first aperture can be angled at about 15° and the longitudinal centerline of the second aperture can be angled at about 45°.

A system including the stem of any of the preceding paragraphs and/or any of the stem described herein is disclosed. Each aperture of the plurality of apertures can include a first opening, a second opening, and a length measured along a longitudinal centerline therebetween. The system can include at least one plug adapted to be received by one or more apertures of the plurality of apertures of the stem.

The system of the preceding paragraphs or as described further herein can also include one or more of the following features. The at least one plug can include at least one elongate plug. A width of the at least one elongate plug can be less than a length thereof. The length of the one or more apertures of the plurality of apertures can be less than the length of the at least one elongate plug. Alternatively, the length of the one or more apertures of the plurality of apertures can be equal to or greater than the length of the at least one elongate plug. The one or more apertures of the plurality of apertures can be adapted to receive the at least one elongate plug along the entire length of the one or more apertures. A width of the at least one plug can be greater than a length thereof. The length of the one or more apertures of the plurality of apertures can be greater than the length of the at least one plug. Two or more of the plugs can be adapted to be inserted into an aperture of the one or more apertures along the longitudinal centerline.

A kit including the stem of any of the preceding paragraphs and/or any of the stem described herein is disclosed. The kit can include a reverse insert, an anatomical articular component, and/or a spacer. The reverse insert can have a proximal portion and a distal portion. The proximal portion of the reverse insert can include a concave surface configured to receive a glenosphere. The distal portion can include a protrusion. The reverse insert can be adapted to directly couple to the stem. The anatomical articular component can have a proximal portion and a distal portion. The proximal portion of the anatomical articular component can include a convex surface. The distal portion of the anatomical articular component can include a protrusion. The anatomical articular component can be adapted to directly couple to the stem. The spacer can include a proximal portion and a distal portion. The spacer can be adapted to couple the reverse insert or the anatomical articular component to the stem. The proximal portion of the spacer can be symmetric or asymmetric.

In some aspects of the disclosure, a kit for a shoulder prosthesis is disclosed. The kit can include a stem, a reverse insert, an anatomical articular component, and/or a spacer. The stem can include a distal shaft portion, a proximal portion, and a metaphyseal portion. The distal shaft portion can be adapted to be anchored in a medullary canal of a humerus. The proximal portion can have a stem face. The metaphyseal portion can include a medial portion and first and second lateral arms. Alternatively, the metaphyseal portion can include only one lateral arm or more than two lateral arms. The first and second lateral arms can extend between and connect the distal shaft portion and the proximal portion. The reverse insert can have a proximal portion and a distal portion. The proximal portion of the reverse insert can include a concave surface adapted to receive a glenosphere. The distal portion of the reverse insert can include a protrusion. The reverse insert can be adapted to directly couple to the stem face. The anatomical articular component can have a proximal portion and a distal portion. The proximal portion of the anatomical articular component can include a convex surface. The distal portion of the anatomical articular component can include a protrusion. The anatomical articular component can be adapted to directly couple to the stem face. The spacer can include a proximal portion, a distal portion, and a protrusion. The protrusion of the spacer can extend from a distal facing surface of the spacer. The spacer can be adapted to couple the reverse insert or the anatomical articular component to the stem. The proximal portion of the spacer can be asymmetric. The protrusion can be adapted to provide rotational alignment between the spacer and the stem.

The kit of the preceding paragraphs or as described further herein can also include one or more of the following features. The proximal portion of the spacer can be symmetric. The stem face can include a central cavity. The central cavity of the stem face can be adapted to receive the reverse insert or the anatomical articular component. The distal shaft portion of the stem can include a plurality of apertures. The plurality of apertures of the distal shaft portion can be adapted to receive a screw or a plug. The spacer can include an engagement feature. The engagement feature can project from the distal facing surface of the spacer. The engagement feature can extend distally of the protrusion. The distal portion of the spacer can include first and second lateral cutouts. The first cutout can be positioned opposite the second cutout. Alternatively, the spacer can include a single lateral cutout or more than two lateral cutouts. The proximal portion of the spacer can include a proximal edge and a distal edge. The proximal edge can be angled relative to the distal edge. The proximal edge can be angled about 5° relative to the distal edge of the proximal portion of the spacer. Alternatively, the proximal edge can be angled less than or more than about 5° relative to the distal edge of the proximal portion of the spacer. For example, the proximal edge can be angled about 3° or about 10°. Alternatively, the distal edge can be angled relative to the proximal edge. The kit can further include a second stem. A distal shaft portion of the second stem can be longer than the distal shaft portion of the first stem. The kit can further include a plug or a plurality of plugs. The plug can be adapted to be received by one of the plurality of apertures. The plug can include a polyethylene material or any suitable material. For example, the plug can include a bone graft.

The kit of the preceding paragraphs or as described further herein can also include one or more of the following features. The kit can further include a second spacer. The second spacer can include a proximal portion and a distal portion. The second spacer can be adapted to couple the reverse insert or the anatomical articular component to the stem.

In some aspects of the disclosure, a stem for a shoulder prosthesis is disclosed. The stem can include a distal shaft portion, a proximal portion, a metaphyseal portion, and a suture groove. The distal shaft portion can be adapted to be anchored in a medullary canal of a humerus. The proximal portion can have a stem face. The stem face can be surrounded by a proximal rim. The metaphyseal portion can include a medial portion and first and second lateral arms. Alternatively, the metaphyseal portion can include only one lateral arm or more than two lateral arms. The first and second lateral arms can extend between and connect the distal shaft portion and the base portion of the proximal portion. The suture groove can be adapted to engage a suture. The suture groove can extend between the proximal rim and the metaphyseal portion along a medial side of the proximal portion. The suture groove can also extend around at least a portion of a circumference of the proximal portion. The suture groove can include a first concave curvature, a second concave curvature, and a convex portion. The second concave curvature can be distal to the first curvature. The convex portion can be positioned between the first concave curvature and the second concave curvature.

The stem of the preceding paragraphs or as described further herein can also include one or more of the following features. A height of the suture groove can be between about 0.5 cm and about 1.0 cm. The stem can further include a plurality of grooves. The plurality of grooves can be positioned on a lateral side of the proximal portion. The plurality of grooves can extend in an anterior-posterior direction. The stem can further include a plurality of grooves on lateral surfaces of the first and second lateral arms of the metaphyseal portion.

In some aspects of the disclosure, a stem for a shoulder prosthesis is disclosed. The stem can include a distal shaft portion, a proximal portion, a metaphyseal portion, and an aperture or a plurality of apertures. The distal shaft portion can be adapted to be anchored in a medullary canal of a humerus. The proximal portion can have a stem face. The stem face can include a central recess, a peripheral wall, and a base portion. The peripheral wall can be positioned along a periphery of the central recess. The base portion can be positioned distal to the peripheral wall. The metaphyseal portion can include a medial portion and first and second lateral arms. Alternatively, the metaphyseal portion can include one lateral arm or more than two lateral arms. The first and second lateral arms can extend between and connect the distal shaft portion and the base portion of the proximal portion. The medial portion can include an arm or a plurality of arms. The arm can have a lateral edge. The first and second lateral arms can have medial edges. A fenestration or a plurality of fenestrations can be defined between the lateral edge of the medial arm and the medial edges of the first and second lateral arms. The aperture can be adapted to receive a screw or a plug. The aperture can be positioned distal to the fenestration of the metaphyseal portion and extend in an anterior-posterior direction.

The stem of the preceding paragraphs or as described further herein can also include one or more of the following features. A longitudinal centerline of the aperture can be less than about 1.0 cm from a distal edge of the fenestration. The aperture can include a circular cross-section. The stem can further include additional apertures. The additional apertures can be positioned distal to the aperture. Each of the additional apertures can be adapted to receive a screw or a plug.

In some aspects of the disclosure, a kit for a shoulder prosthesis is disclosed. The kit can include a stem, a stem holder, and a jig. The stem can be adapted to be implanted into a shoulder of a patient. The stem can include a proximal portion and a plurality of apertures. The proximal portion can have a stem face. Each of the plurality of apertures can be adapted to receive cement or a screw to secure the stem within the shoulder of the patient. The stem holder can be adapted to implant the stem into the shoulder of the patient when the stem is being secured with the cement. The jig can be adapted to implant the stem into the shoulder of the patient when the stem is being secured with one or more screws.

The kit of the preceding paragraphs or as described further herein can also include one or more of the following features. The kit can further include a second stem. The second stem can include a second length and a plurality of apertures. The stem can include a first length less than the second length. The jig can be adapted to implant the second stem into the shoulder of the patient when the second stem is being secured with the one or more screws. The jig can include a distal arm extension adapted to guide the one or more screws into one or more apertures of a plurality of apertures of the second stem. The distal arm extension of the jig can be adapted to be moveable between a first side of the jig and a second side of the jig. The distal arm extension can be positioned on the first side of the jig to implant the second stem into a left shoulder of the patient. The distal arm extension can be positioned on the second side of the jig to implant the second stem into a right shoulder of the patient. The jig can include an interfacing portion. The interfacing portion can be adapted to be removably coupled to the stem face of the second stem. The jig can include an impaction head. The impaction head of the jig can be adapted to receive impaction forces from a tool to implant the second stem into the shoulder of the patient. The impaction head of the jig can be located proximal to the interfacing portion. The jig can include a height gauge. The height gauge of the jig can be adapted to determine a height positioning of the second stem when implanting the second stem into the shoulder of the patient. The stem holder can include an impaction head. The impaction head of the stem holder can be adapted to receive impaction forces from a tool to implant the stem into the shoulder of the patient. The stem holder can include a height gauge. The height gauge of the stem holder can be adapted to determine a height positioning of the stem when implanting the stem into the shoulder of the patient.

In some aspects of the disclosure, a system for implanting a shoulder prosthesis is disclosed. The system can include a stem and a jig. The stem can be adapted to be implanted into a shoulder of a patient. The stem can include a plurality of apertures. The plurality of apertures can be adapted to receive one or more screws to secure the stem within the shoulder of the patient. The jig can be adapted to introduce the stem into the shoulder of the patient. The jig can include a distal arm extension. The distal arm extension can be adapted to guide the one or more screws into one or more apertures of the plurality of apertures of the stem. The distal arm extension of the jig can be adapted to be moveable between a first side of the jig and a second side of the jig. The distal arm extension can be positioned on the first side of the jig to implant the stem into a left shoulder of the patient. The distal arm extension can be positioned on the second side of the jig to implant the stem into a right shoulder of the patient. The distal arm extension can include a screw guide. The screw guide can be adapted to align the one or more screws with the one or more apertures of the stem. The screw guide can include a first aperture, a second aperture, and a sliding plate. Alternatively, the screw guide can include only a single aperture or more than two apertures. The sliding plate can be adapted to cover the first or second aperture of the screw guide. The first aperture of the screw guide can be adapted to align a screw of the one or more screws with a first aperture of the one or more apertures of the stem when the distal arm extension is on the first side of the jig. The second aperture of the screw guide can be adapted to align the screw of the one or more screws with a second aperture of the one or more apertures of the stem when the distal arm extension is on the second side of the jig. The sliding plate can cover the first aperture of the screw guide when the distal arm extension is on the second side of the jig. The sliding plate can cover the second aperture of the screw guide when the distal arm extension is on the first side of the jig.

The system of the preceding paragraphs or as described further herein can also include one or more of the following features. The jig can further include an inserter portion. The inserter portion can include an impaction head. The impaction head can be adapted to receive impaction forces from a tool. The inserter portion can further include an interfacing portion. The interfacing portion can be adapted to removably couple to a proximal portion of the stem. The jig can further include a height gauge. The height gauge can be adapted to determine a height positioning of the stem when implanting the stem into the shoulder of the patient. The jig can further include a vertical support structure. The vertical support structure can extend between the height gauge and the distal arm extension. The vertical support structure can include a proximal end and a distal end. The distal arm extension can be adapted to rotate about the distal end of the vertical support structure to move between the first and second sides of the jig. The distal arm extension can include a first portion and a second portion. The first portion of the distal arm extension can be coupled to the distal end of the vertical support structure and extend radially outward from a longitudinal axis of the jig. The second portion of the distal arm extension can include the screw guide and a second screw guide. The second screw guide can include an aperture. The aperture can be adapted to align a second screw of the one or more screws with a third aperture of the one or more apertures of the stem. The second screw guide can include an aperture adapted to align a second screw of the one or more screws with a third aperture of the one or more apertures of the stem. The first aperture of the one or more apertures of the stem can be adjacent a distal tip of the stem. The second aperture of the one or more apertures of the stem can be proximal to the first aperture of the one or more apertures of the stem. The third aperture of the one or more apertures of the stem can be proximal to the second aperture of the one or more apertures of the stem. The distal arm extension can include a curvature or a bend. The curvature or bend can extend between the first portion and the second portion to align the screw guide and the second screw guide with the one or more apertures of the stem. The sliding plate can move between a first position and a second position along a longitudinal axis of the screw guide. The sliding plate can be in the first position when the distal arm extension is on the first side of the jig. The sliding plate can be in the second position when the distal arm extension is on the second side of the jig. The sliding plate can be adapted to move between the first and second positions by gravitational forces. Alternatively, the sliding plate can be adapted to move between the first and second positions by other forces. For example, a user can manually move the sliding plate between the first and second positions.

In some aspects of the disclosure, a method for positioning a stem for a shoulder prosthesis into a medullary canal of a humerus of a patient is disclosed. The method can include: attaching a stem face of a stem to an interfacing portion of a stem holder; inserting the stem into the medullary canal of the humerus; and securing the stem in the medullary canal of the humerus. The stem can include a proximal portion and a distal shaft portion. The proximal portion can have the stem face. The distal shaft portion can have a plurality of apertures.

The method of the preceding paragraphs or as described further herein can also include one or more of the following features. The method can further include: inserting a plug into an aperture of the plurality of apertures of the stem and cutting the length of the plug. The aperture can include a first opening, a second opening, and a length measured along a longitudinal centerline therebetween. The plug can include a length and a width. The width can be less than the length of the plug. The length of the plug can be greater than the length of the aperture. The method can further include: inserting a first plug into one of the plurality of apertures of the stem; and inserting a second plug into said one of the plurality of apertures of the stem. The aperture can include a first opening, a second opening, and a length measured along a longitudinal centerline therebetween. Each of the first and second plugs can include a length and a width. The width can be greater than the length of each of the first and second plugs. The length of the aperture can be greater than the length of each of the first and second plugs. Securing the stem can include providing bone cement in the medullary canal of the humerus. The method can further include applying impaction forces to an impaction head of the stem holder. Securing the stem in the medullary canal can include inserting a screw into one of the plurality of apertures of the stem. The method can further include aligning a screw guide of the stem holder with the plurality of apertures. The screw guide can be carried by a distal arm extension of the stem holder. The method can further include: positioning the distal arm extension of the stem holder on a first side of the stem holder when the stem is inserted into the humerus of a left shoulder and positioning the distal arm extension of the stem holder on a second side of the stem holder when the stem is inserted into the humerus of a right shoulder. When the distal arm extension is on the second side of the stem holder, the distal arm extension can be inverted compared to when the distal arm extension is on the first side of the stem holder. When the distal arm extension is on the first side of the stem holder, the screw guide can cover a first aperture of the plurality of apertures. When the distal arm extension is on the second side of the stem holder, the screw guide can cover a second aperture of the plurality of apertures. The method can further include sliding a plate on the screw guide to a first position to cover the first aperture or a second position to cover the second aperture of the plurality of apertures. The plate can slide by gravitational forces. Alternatively, a user can manually move the sliding plate between the first and second positions.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No aspects of this disclosure are essential or indispensable.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments. The following is a brief description of each of the drawings.

FIG. 1A illustrates an anatomical shoulder prosthesis disposed in the humerus of a shoulder;

FIG. 1B illustrates a reverse shoulder prosthesis disposed in the humerus of a shoulder;

FIG. 2 illustrates a schematic diagram of a shoulder arthroplasty system including an arthroplasty kit that can be used in performing anatomic or reverse arthroplasty, in converting from one of anatomic to reverse, or reverse to anatomic arthroplasty;

FIG. 3 illustrates a shoulder arthroplasty system for fracture repair;

FIG. 4A is a perspective view of a reverse shoulder prosthesis;

FIG. 4B is a sideview of a reverse shoulder prosthesis with symmetric spacer;

FIG. 4C is a sideview of a reverse shoulder prosthesis with asymmetric spacer;

FIG. 5A is a perspective view of a humeral stem that is used in the shoulder prosthesis of FIG. 3;

FIG. 5B is a plan view of the stem of FIG. 5A;

FIG. 5C is another plan view of the stem of FIG. 5A;

FIG. 5D is another plan side view of the stem of FIG. 5A;

FIG. 5E is another plan side view of the stem of FIG. 5A;

FIG. 5F is a plan view of the stem of FIG. 5A illustrating different dimensions;

FIG. 5G is another plan view of the stem of FIG. 5A illustrating different dimensions;

FIG. 6 is a cross-sectional view of the stem of FIG. 5A taken through the section plane 6-6 in FIG. 5D;

FIGS. 7A and 7B illustrate a plan view (FIG. 7A) and a perspective view (FIG. 7B) of a face of the stem of FIG. 5A, the face being configured to couple with an anatomic insert to form the anatomic shoulder prosthesis of FIG. 1A and with a reverse insert to form the reverse shoulder prosthesis of FIG. 1B;

FIG. 8A is a perspective view of another embodiment of a humeral stem;

FIG. 8B is a plan view of the stem of FIG. 8A;

FIG. 8C is another plan view of the stem of FIG. 8A;

FIG. 8D is another plan view of the stem of FIG. 8A;

FIG. 8E is another plan view of the stem of FIG. 8A;

FIG. 8F is a cross-sectional view of the stem of FIG. 8A taken through the section plane 8F-8F in FIG. 8D;

FIG. 9A is a bottom perspective view of a spacer that can be used with a humeral stem;

FIG. 9B is a side view of the spacer shown in FIG. 9A;

FIG. 9C is a top perspective view of the spacer shown in FIG. 9A;

FIG. 10A is a bottom perspective view of another spacer that can be used with a humeral stem;

FIG. 10B is a side view of the spacer shown in FIG. 10A;

FIG. 10C is a top perspective view of the spacer shown in FIG. 10A;

FIG. 11A is a bottom perspective view of yet another spacer that can be used with a humeral stem;

FIG. 11B is a side view of the spacer shown in FIG. 11A;

FIG. 11C is a top view of the spacer shown in FIG. 11A;

FIG. 12A is a top perspective view of an anatomic adaptor that can couple an anatomic insert to a humeral stem to form an anatomic shoulder prosthesis;

FIG. 12B is a bottom perspective view of the anatomic adaptor shown in FIG. 12A;

FIG. 12C is a side view of the anatomic adaptor shown in FIG. 12A;

FIGS. 13A-13B illustrate a screw that can be used to secure a humeral stem into the humerus of a shoulder;

FIG. 14 is a perspective view of the humeral stem shown in FIG. 5A with a plurality of plugs in the plurality of apertures of the humeral stem;

FIG. 15 is a plug that can be received by the plurality of apertures of a humeral stem, as shown in FIG. 14;

FIG. 16 is another plug that can be received by the plurality of apertures of a humeral stem, as shown in FIG. 14;

FIG. 17A is a perspective view of a graft tool;

FIG. 17B is a cross-sectional view of the graft tool shown in FIG. 17A;

FIG. 17C is a perspective view of a tip of the graft tool shown in FIG. 17A;

FIG. 17D is a side view of the tip of the graft tool shown in FIG. 17A;

FIG. 17E is another side view of the tip of the graft tool shown in FIG. 17A;

FIG. 17F is a cross-sectional view of the tip of the graft tool shown in FIG. 17A;

FIG. 17G is a cross-sectional view of a tool inserted into the tip of the graft tool shown in FIG. 17A;

FIG. 18A is a top perspective view of a stem holder;

FIG. 18B is bottom perspective view of the stem holder shown in FIG. 18A;

FIG. 18C is a side view of a moveable assembly of the stem holder shown in FIG. 18A;

FIG. 18D is a partially transparent side view of the stem holder shown in FIG. 18A;

FIG. 18E is a perspective view of the stem holder shown in FIG. 18A and the humeral stem shown in FIG. 5A;

FIG. 18F is another perspective view of the stem holder and the humeral stem shown in FIG. 18E;

FIG. 18G is a side view of the stem holder and the humeral stem shown in FIG. 18E;

FIG. 18H is another side view of the stem holder and the humeral stem shown in FIG. 18E;

FIG. 18I illustrates the stem holder and the humeral stem shown in FIG. 18E with the humeral stem disposed in a humerus of a shoulder;

FIG. 19A is a perspective view of a jig and the humeral stem shown in FIG. 5A;

FIG. 19B is another perspective view of the jig and the humeral stem shown in FIG. 19A;

FIG. 19C is a bottom perspective view of the jig shown in FIG. 19A;

FIG. 19D is a side view of a moveable assembly of the jig shown in FIG. 19A;

FIG. 19E is a partially transparent side view of the jig shown in FIG. 19A;

FIG. 19F illustrates the jig and the humeral stem shown in FIG. 19A with the humeral stem disposed in a humerus of a shoulder;

FIG. 20A is a perspective view of a first configuration of another jig and the humeral stem shown in FIG. 8;

FIG. 20B is another perspective view of the first configuration of the jig and the humeral stem shown in FIG. 20A;

FIG. 20C is a side view of the first configuration of the jig and the humeral stem shown in FIG. 20A;

FIG. 20D is another perspective view of the first configuration of the jig and the humeral stem shown in FIG. 20A;

FIG. 20E illustrates the first configuration of the jig and the humeral stem shown in FIG. 20A with the humeral stem disposed in a humerus of a shoulder;

FIG. 21A is a perspective view of a second configuration of the jig and the humeral stem shown in FIG. 20A;

FIG. 21B is another perspective view of the second configuration of the jig and the humeral stem shown in FIG. 21A;

FIG. 21C is a side view of the second configuration of the jig and the humeral stem shown in FIG. 21A;

FIG. 21D is another perspective view of the second configuration of the jig and the humeral stem shown in FIG. 21A; and

FIG. 21E illustrates the second configuration of the jig and the humeral stem shown in FIG. 21A with the humeral stem disposed in a humerus of a shoulder.

### DETAILED DESCRIPTION

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

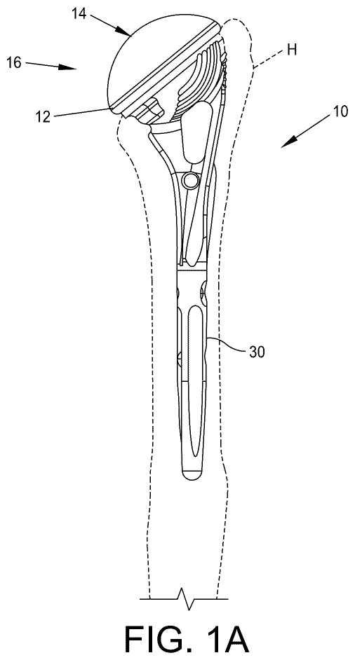

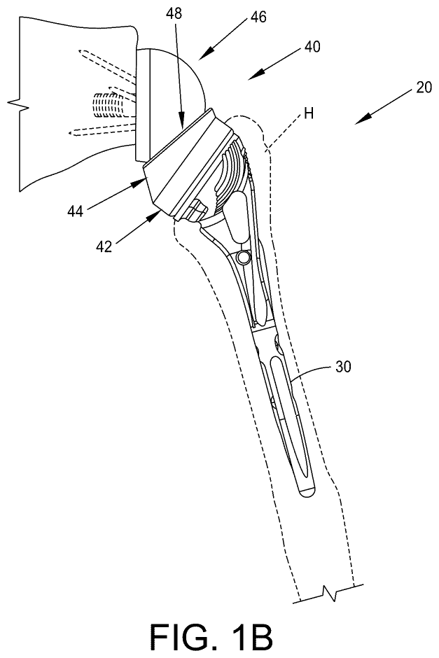

FIGS. 1A and 1B show two conventional approaches to total shoulder arthroplasty. FIG. 1A shows an implant system 10 of an anatomic approach in which a natural humeral head of a natural human humerus H is replaced with an anatomical insert or articular component 16 that includes an articular body 12 having a convex articular surface 14. In some configurations, the glenoid of the scapula can be modified with an implant providing a concave surface for articulation of the humeral articular body 12. The humeral articular body 12 is secured to the humerus H using a humeral stem 30. The humeral articular body 12 may be secured to the humeral stem 30 using an adaptor.

FIG. 1B shows an implant system 20 of a reverse approach in which the humerus H is fitted with a reverse insert or articular component 40 including an articular body 44 having a concave articular surface 48. The glenoid region of the scapula is fitted with a spherical articular body, commonly called a glenosphere 46. In this case, the concave articular surface 48 placed on the humerus H articulates the glenosphere 46, which is fixed relative to the scapula. The reverse articular body 44 is mounted to a spacer 42 that is disposed between the reverse humeral articular body 44 and a humeral stem 30 that is surgically implanted in the humerus H. The humerus H is prepared by providing access to the medullary canal of the humerus H.

One can see that the anatomic and reverse approaches generally use different hardware to secure the articular components. For example, the presence of the spacer 42 may require more joint space. Thus, the reverse configuration may only be suitable for some patients with large joint space or following more invasive preparation of the humerus and/or the scapula.

With a standard shoulder prosthesis, when a surgeon desires to convert a primary anatomic shoulder prosthesis into a reverse shoulder prosthesis, the surgeon must typically remove the entire prosthesis, thereby risking further weakening the bone. However, using a modular system such as implant systems 10 and 20 illustrated in FIGS. 1A and 1B, the surgeon may leave the stem 30 implanted in the bone and simply replace the anatomic insert 16 with the reverse insert 40. For example, the anatomic insert 16 may be removed using a wedge or similar instrument. The reverse insert 40 can be inserted into direct engagement with the stem face of the humeral stem 30, for example, impacted into place using an impactor or similar tool.

### I. Systems and Kits with Shared Implant Components

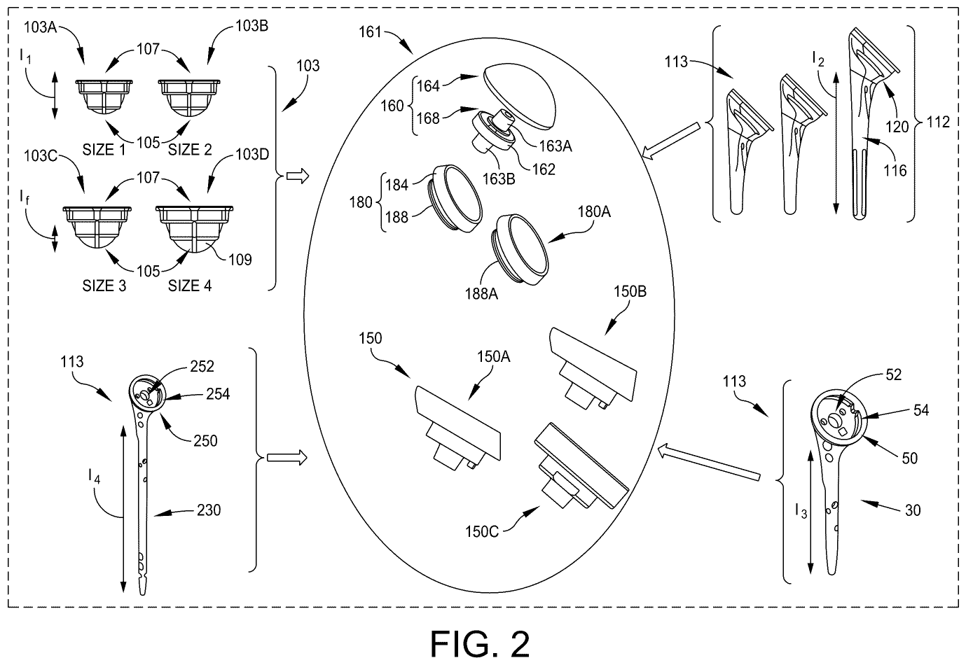

FIG. 2 is a schematic diagram of a total arthroplasty system comprising an arthroplasty kit 100 that can be used to perform anatomic or reverse arthroplasty, or to convert from one of anatomic to reverse or reverse to anatomic arthroplasty, according to various embodiments. The kit 100 can comprise one or a plurality of stemless humeral anchors 103, one or a plurality of stemmed humeral anchors 113, one or a plurality of articular components 161, and/or one or more spacers and adapters 150 configured to couple a stemless humeral anchor 103 or a stemmed humeral anchor 113 with an anatomic insert or a reverse insert. The stemless humeral anchors 103 can have a distal portion 105 and a proximal portion 107. The distal portion 105 of the anchors 103 shown in FIG. 2 can have one or a plurality of fins 109 extending distally. The fins 109 can be configured to secure the anchors 103 into the humerus. The stemless anchors 103 can have a tapered profile in which the anchors 103 narrow from the proximal portion 107 to the distal portion 105.

As shown in FIG. 2, the stemless anchors 103 can be provided in a plurality of sizes to accommodate patients of different sizes, different degrees of bone damage to the humerus, etc. In some embodiments, the lateral size of the stemless anchors 103 may vary so as to fit within different-sized resections of the humerus. For example, the kit 100 can comprise a plurality of stemless anchors 103A, 103B, 103C, 103D . . . 103n, with n being the number of different sizes. Although four sizes are illustrated in FIG. 2 (e.g., n=4, with anchors 103A-103D), in other embodiments, the kit can include any suitable number of anchors. In some embodiments, a length l₁ of the stemless anchors 103A-103D may also vary so as to extend into the humerus by a depth that the clinician selects based on the particular patient being treated. Furthermore, the anchors 103A-103D can have different fin lengths l_{f} of the fins 109 to accommodate different sizes of the humerus.

In various embodiments, the fin lengths l_{f} of the anchors 103A-103D can differ substantially so as to beneficially provide a wide range of anchor strengths to the humerus and accommodate patients with different levels of bone damage. In the arrangement of FIG. 2, for example, the first anchor 103A can have the shortest overall length l₁ and the shortest overall fin length l_{f}. The fourth anchor 103D can have the longest overall length l₁ and the longest overall fin length l_{f}. In various embodiments, a ratio of an overall length l₁ of one anchor 103 (for example, the largest anchor 103) to an overall length l₁ of another anchor 103 (for example, the smallest anchor 103) in the kit 100 can be in a range of 1.15 to 2.5, in a range of 1.18 to 2.5, in a range of 1.2 to 2.5, in a range of 1.2 to 2, in a range of 1.2 to 1.8, in a range of 1.2 to 1.6, in a range of 1.3 to 1.6, or in a range of 1.25 to 1.4.

The kit 100 can also include one or a plurality of stemmed humeral anchors 113. The kit 100 can include one or more humeral stem anchors 112, each of which includes a proximal metaphysis portion 120 and an elongate diaphysis portion 116 extending therefrom. The diaphysis portion 116 is sometimes referred to herein as a stem or stem portion. The stemmed humeral anchors 113 may be used in patients in which stemless anchors 103 may not be adequately secured to the humerus, for example, in patients that have experienced severe bone loss. As with the stemless anchors 103, the kit 100 can include humeral stem anchors 113 (sometimes referred to herein as a stemmed anchor) having a plurality of different sizes, e.g., different lateral sizes and/or different lengths l₂. For example, as shown in FIG. 2, the stemmed humeral anchors 113 can have respective lengths l₂ that are longer than the lengths l₁ of the stemless anchors 103. Beneficially, the inclusion of differently-sized stemmed anchors 113 in the kit 100 can enable the clinician to select the appropriate size for a particular patient to ensure a secure implant of the anchor 113 into the patient, in view of the patient's bone size and health. In various embodiments, the lengths l₂ of the stemmed humeral anchors can be in a range of about 55 mm and about 175 mm. By contrast, the shorter lengths l₁ of the stemless humeral anchors 103 can be in a range of about 16 mm and about 28 mm. In various embodiments, stemmed humeral anchors 113 can be configured to reach into the intramedullary canal of the humerus H for additional anchorage.

In some embodiments, the stemmed humeral anchors 113 can include trauma or fracture stem anchors or humeral stems 30, 230, which can be used in patients that have experienced a fracture of the humerus H. The trauma or fracture stems 30, 230 may be used where the humerus has fractured into one or more pieces. Moreover, the shaft portions of the fracture stems 30, 230 may also have respective length l₃, l₄ such that the length l₄ of the shaft portion of the longer fracture stem 230 can be longer than the length l₃ of the shaft portion of the shorter fracture stem 30. In various embodiments, the lengths l₄ of the shaft portion of the longer fracture stem 230 can be in a range of about 125 mm and about 175 mm, in a range of about 150 mm and about 175 mm, or about 168 mm. By contrast, the shorter lengths l₃ of the shaft portion of the shorter fracture stem 30 can be in a range of about 50 mm and about 100 mm, a range of about 75 mm and about 100 mm, or about 88 mm.

Beneficially, the kit 100 can comprise one or a plurality of shared humeral components that be used with either the stemless humeral implants 103 or the stemmed humeral implants 113, depending on which implant 103 or 113 would be more appropriate for a particular patient's humeral anatomy. For example, the shared humeral components of the kit 100 can comprise a plurality of articular components or assemblies 161 that can be used in conjunction with either the stemless implants 103 or the stemmed implants 113. As explained herein, both the stemless humeral anchors 103 and the stemmed humeral anchors 113 can include shared engagement features that can be used with the same set of tools and/or articular components. For example, as described herein, the stemless anchors 103 and stemmed anchors 113 can include convex and concave locking features configured to engage with the same set of articular components.

For example, the kit 100 can include an anatomic articular component 160 configured to mechanically couple to both the stemless humeral implants 103 and the stemmed humeral implants 113. The clinician may select the anatomic articular component 160 for procedures in which an anatomic reconstruction is suitable. The anatomic articular component 160 can comprise a coupler 168 and an articular body 164 (anatomical) configured to mechanically engage the coupler 168. As shown in FIG. 2, the articular body 164 for the anatomic articular component 160 can comprise a rounded, convex surface configured to engage a glenoid surface of the patient. The coupler 168 can serve to mechanically connect the anatomical articular body 164 (e.g., a rounded or essentially spherical surface) to either a stemless humeral implant 103 or a stemmed humeral implant 113, depending on the patient's humeral bone structure. The articular body 164 and the coupler 168 can comprise a metal, such as cobalt, chrome, or titanium. In some embodiments, the articular body comprises a pyrocarbon layer on at least the articular surface. In various embodiments, the kit 100 can include anatomic articular components 160 having a plurality of sizes.

The kit 100 can also include a reverse articular component 180 configured to mechanically couple to both the stemless humeral implants 103 and the stemmed humeral implants 113. The clinician may select the reverse articular component 180 for procedures in which a reverse anatomic reconstruction is suitable. The reverse articular component 180 can comprise a reverse articular body 184 and a locking device 188 configured to secure the reverse articular component 180 to a stemless humeral implant 103 or a stemmed humeral implant 113, depending on the clinician's recommendation during the procedure. As shown, the reverse articular body 184 can comprise a rounded concave surface (e.g., essentially spherical) configured to engage with a glenosphere connected to the glenoid of the patient (not shown but in some cases combined with the kit into a larger surgical kit). In addition, in some embodiments, the kit 100 can include a wear resistant reverse articular component 180A, which may be generally similar to the reverse articular component 180 but may further be formed to include vitamin E to promote long-term compatibility with the patient's bone structure. The reverse components 180, 180A can comprise a polymer, including, for example, ultra-high molecular weight polyethylene. In various embodiments, the kit 100 can include reverse articular components 180, 180A having a plurality of sizes.

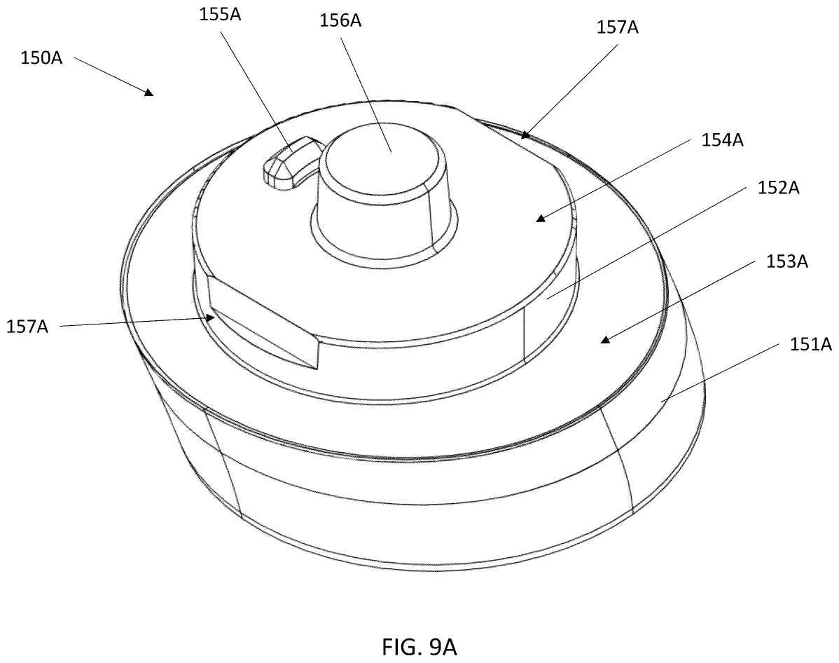

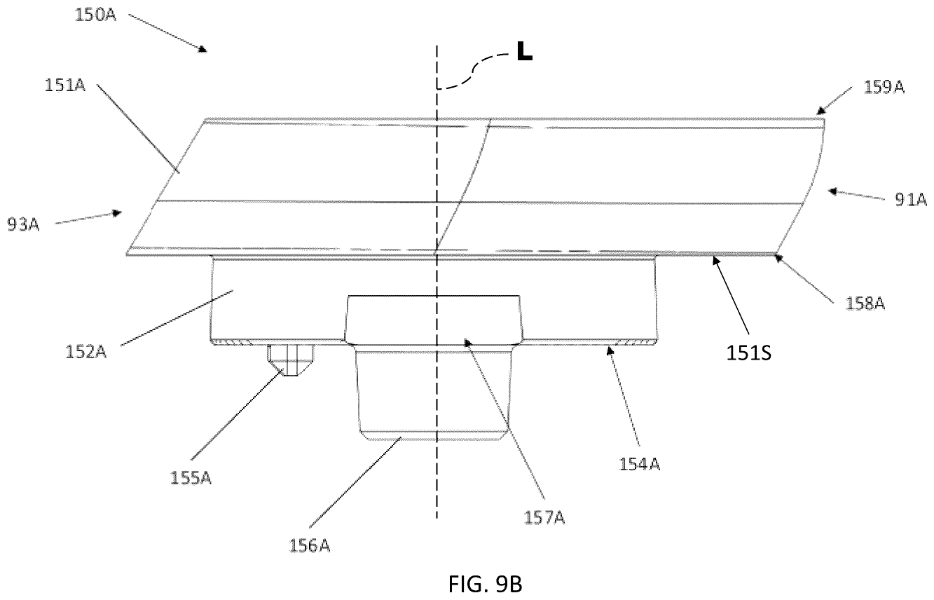

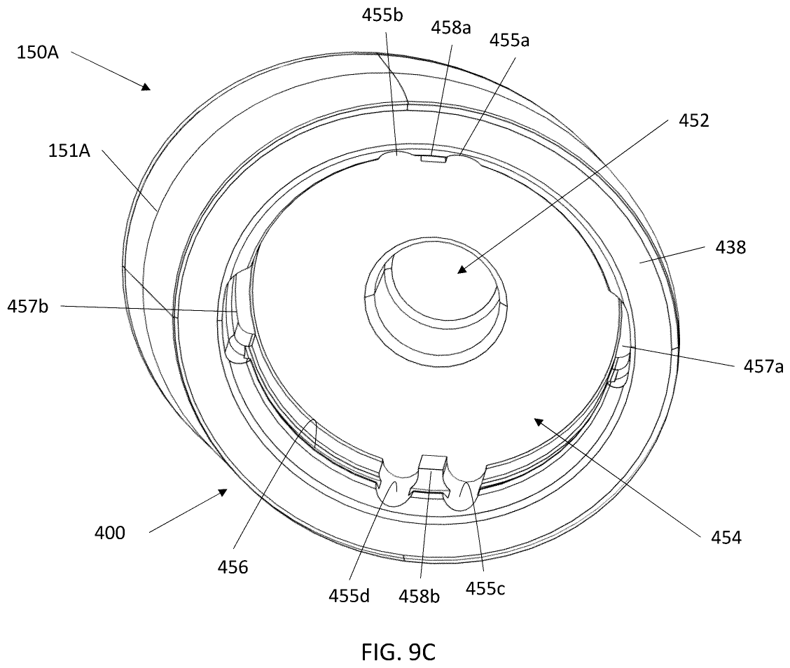

The kit 100 can also include one or more spacers 150 that can mechanically couple the reverse articular component 180 or the anatomical articular component 160 to the stemless humeral implants 103 or the stemmed humeral implants 113. As shown in FIG. 2, the one or more spacers 150 can be provided in a plurality of sizes to accommodate patients of different sizes, different degrees of bone damage to the humerus, etc. For example, the kit 100 can comprise a plurality of spacers 150A, 150B, 150C . . . 150n, with n being the number of different sizes. Although four sizes are illustrated in FIG. 2 (e.g., n=4, with spacers 150A-150C), in other embodiments, the kit can include any suitable number of spacers. Additionally, the one or more spacers 150 can be symmetric or asymmetric. The one or more spacers and adapters 150 are further described below in relation to FIGS. 9A-12C.

During an arthroplasty procedure, the clinician may inspect the bone structure of the humerus and/or the scapula to determine whether the anatomy is suitable for a stemless or stemmed humeral anchor, and whether the anatomy is suitable for an anatomical or reverse anatomical reconstruction. Beneficially, the kit 100 shown in FIG. 2 can provide the clinician with a total arthroplasty system including components that are compatible with stemless or stemmed anchors, and with anatomical or reverse anatomical constructions. For example, during a procedure, the clinician may observe that the patient has sufficient humeral bone structure so that a stemless anchor 103 may be used to reduce the damage to the patient's anatomy. The clinician may also elect whether to proceed with an anatomical reconstruction or a reverse construction, and can accordingly select either the anatomical articular component 160 or the reverse articular component 180, 180A.

Similarly, if during a shoulder arthroplasty procedure, the clinician determines that the patient's bone structure is damaged or otherwise more suited to a stemmed anchor 113, then the clinician can select an appropriately sized stemmed anchor 113. The clinician can further select whether to proceed with an anatomical reconstruction or a reverse construction, and can accordingly select either the anatomical articular component 160 or the reverse articular component 180, 180A. Beneficially, the kit 100 of FIG. 2 includes interchangeable or interoperable components that can be used in stemmed or stemless anchors, and with anatomical or reverse anatomical reconstructions. Because the shared humeral articular components 161 (e.g., anatomical or reverse anatomical articular bodies) can be used with either the stemless or stemmed anchors 103, 113, the clinician can make, or change, reconstruction decisions during surgery. The kit 100 can accordingly enable the clinician to quickly determine the reconstruction procedure most suitable for a patient and can provide the clinician with the components to be used for that reconstruction procedure.

As explained above, for humeral fractures, the kit 100 can also include one or more trauma stems 30, 230. Beneficially, the trauma stem(s) 30, 230 can include engagement features generally similar to or the same as the engagement features in the stemless anchors 103 and humeral stem anchors 113, such that the trauma stem(s) 30, 230 can be used with a common set of shared articular components 161 and tools. Beneficially, therefore, the kit 100 can provide a shared set of implantation tools and a shared set of articular components 161 that can be used with either stemless or stemmed humeral anchors 103, 113, and that can be used for anatomical or reverse anatomical reconstructions.

In some embodiments, the coupler 168 can comprise a proximal extension 163A configured to connect to the articular body 164 and a distal extension 163B. The distal extension 163B for can be received within a first recess 52, 252 of a stem face 50, 250 of the fracture stem 30, 230 for anatomical reconstructions. In some embodiments, the recess 52, 252 is can be recessed from (e.g., extends distally from) a distal end of a second recess 54, 254. In these embodiments, the disc or middle portion 162 can provide a spacer function in use in the trauma stem 30, 230. In some configurations, the recess 52. 252 can be elevated toward the resection plane, and the disc or middle portion 162 disposed between the proximal extension 163A and the distal extension 163B can be eliminated. Additional details of trauma stems may be found throughout International Application No. PCT/US2015/065126, titled “CONVERTIBLE STEM/FRACTURE STEM,” filed Dec. 10, 2015, the entire contents of which are included in the Appendix.

The final implant can take any suitable configuration, such as any that are described in International Application No. PCT/US2019/054007, titled “SHOULDER PROSTHESIS COMPONENTS AND ASSEMBLIES,” and International Application No. PCT/US2019/054023, titled “MODULAR HUMERAL HEAD,” which were filed on Apr. 9, 2020. The final implant can take any configuration as disclosed in International Application No. PCT/US2020/053629, titled “SHOULDER PROSTHESIS COMPONENTS AND ASSEMBLIES,” filed on Sep. 30, 2020. The articular components can take any configuration as disclosed in International Application No. PCT/US2020/053625, titled “REVERSE SHOULDER SYSTEMS,” filed Sep. 30, 2020. The entire contents of each of the applications listed in this paragraph are included in the Appendix.

### II. Examples of Fracture Stems

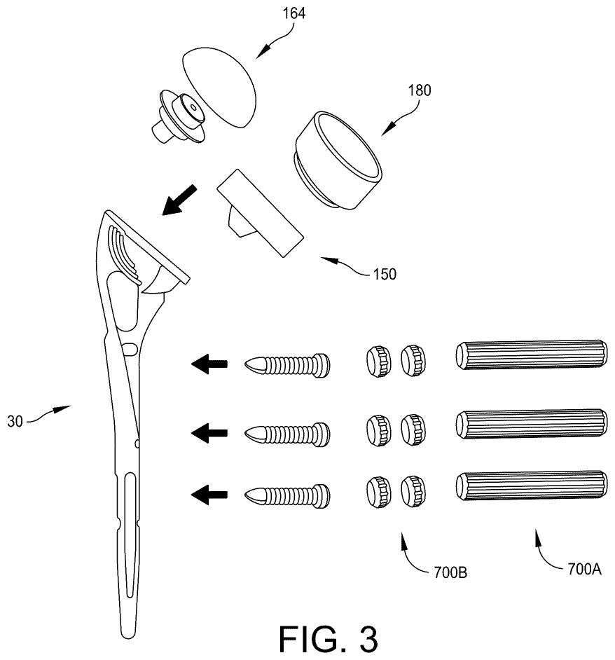

FIG. 3 illustrates a shoulder arthroplasty system for treating a patient with a fractured humerus. The arthroplasty system may include a fracture stem 30, an anatomic articular component 160, a reverse articular component 180, a plurality of screws 170 configured to secure the fracture stem 30 within the humerus of the patient, and/or a plurality of plugs 700A, 700B configured to be received by a plurality of apertures of the stem 30, 230. As discussed above, a clinician may couple the anatomic articular component 160 to the fracture stem 30 when an anatomic reconstruction is suitable. The coupler 168 may mechanically couple the articular body 164 to the stem face 50 of the fracture stem 30.

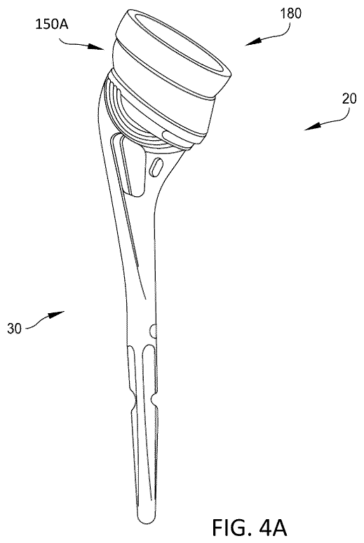

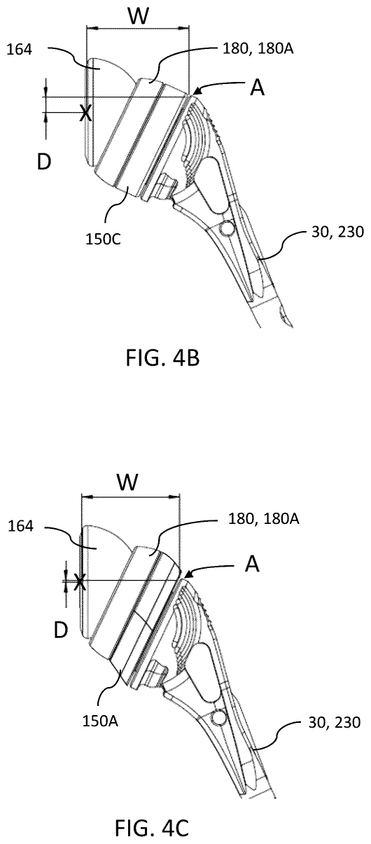

When a reverse reconstruction is suitable, the clinician may couple the reverse articular component 180 to the fracture stem 30. The clinician may directly couple the reverse articular component 180 to the stem face 50 of the fracture stem 30 or the clinician may use a spacer 150 to couple the reverse articular component 180 to the stem face 50 of the fracture stem 30. For example, FIG. 4 illustrates an assembled reverse shoulder prosthesis 20 with a spacer 150A coupling the reverse articular component 180 to the fracture stem 30.

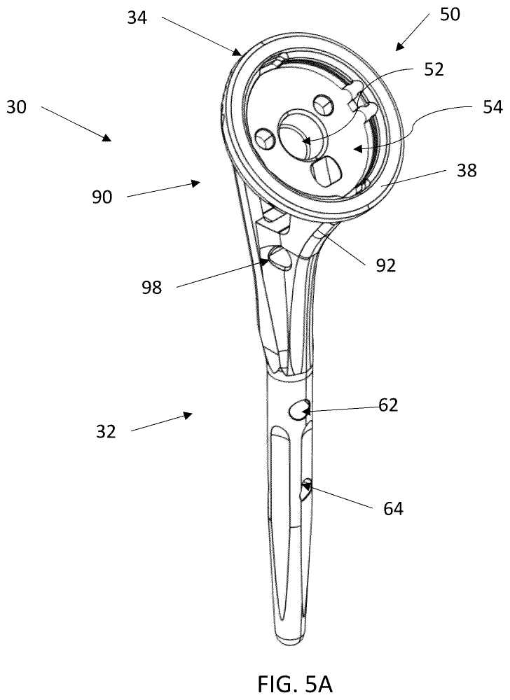

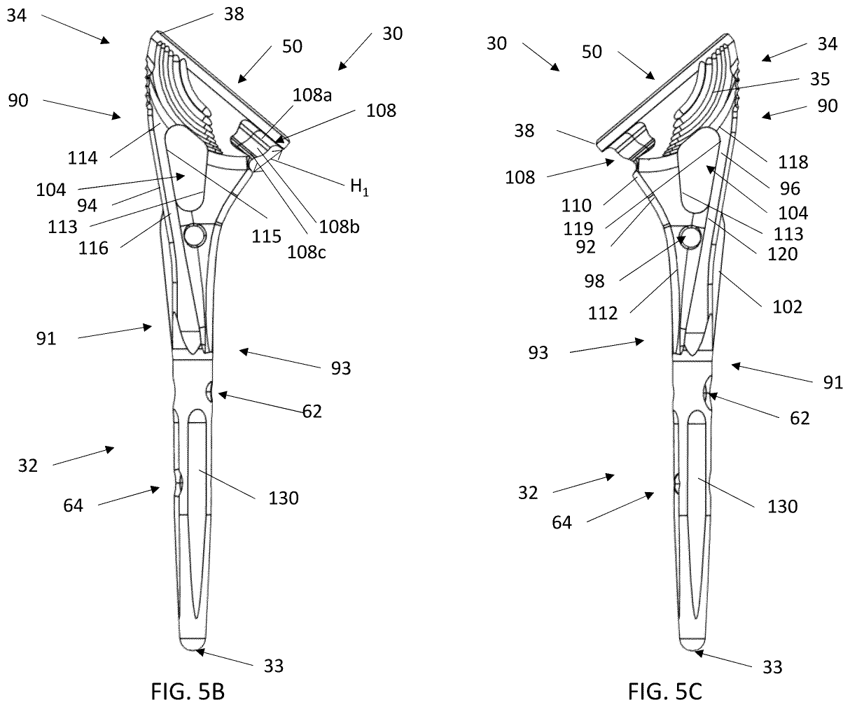

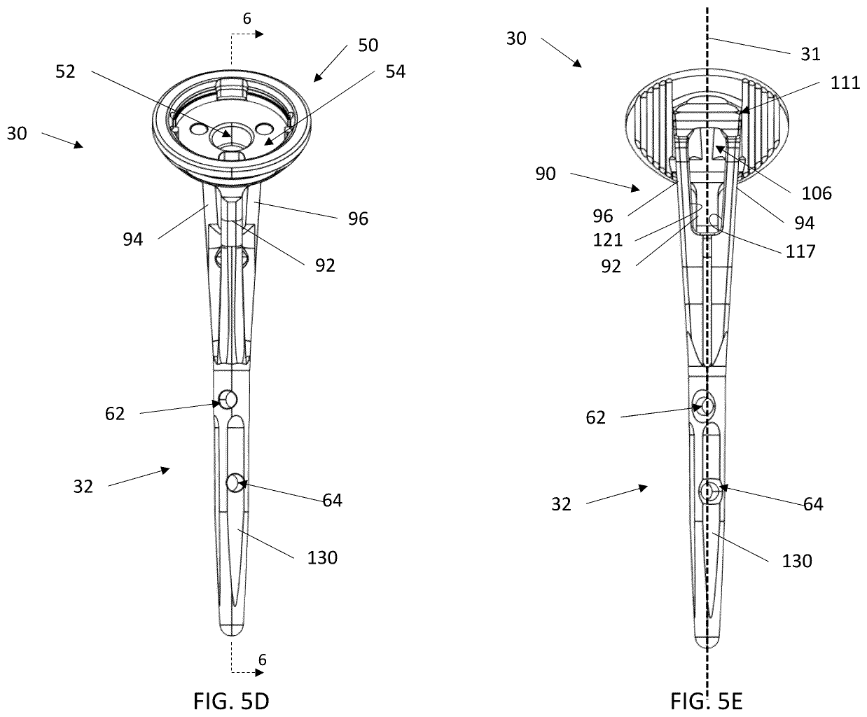

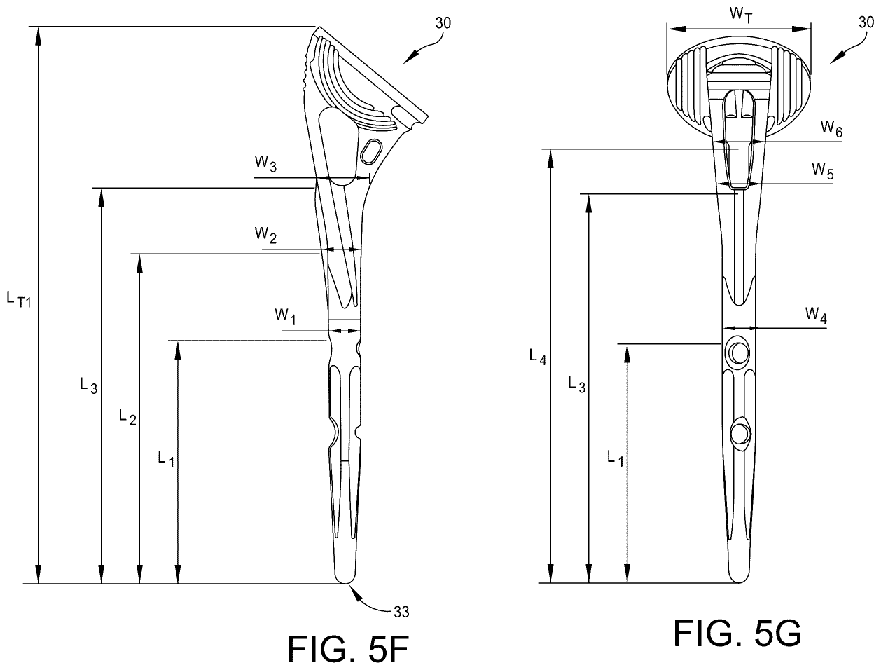

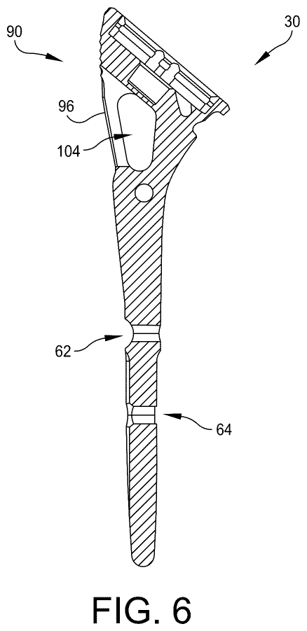

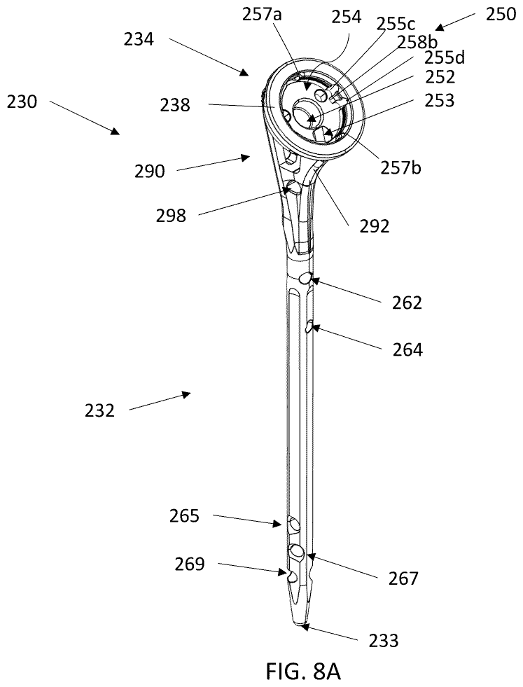

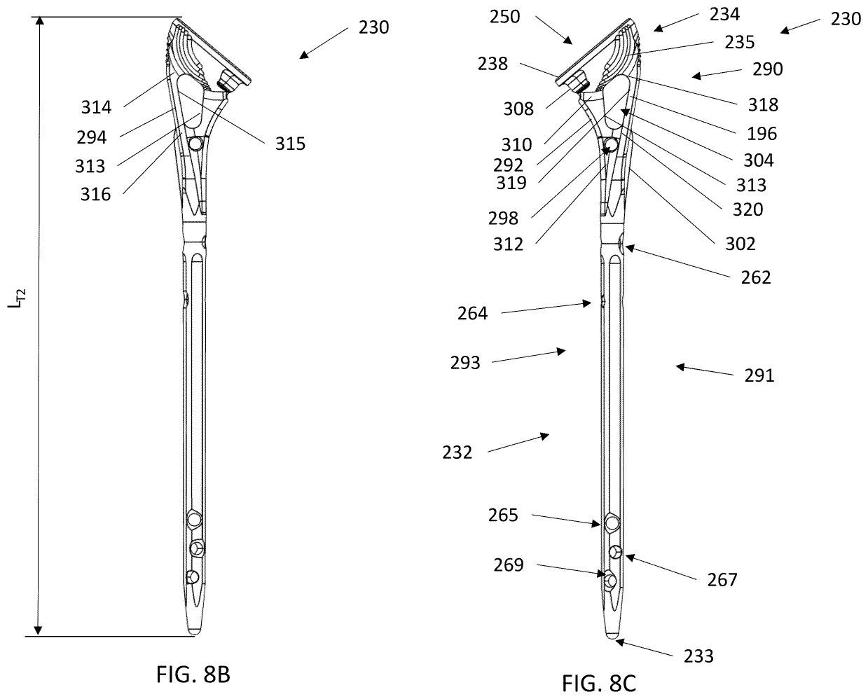

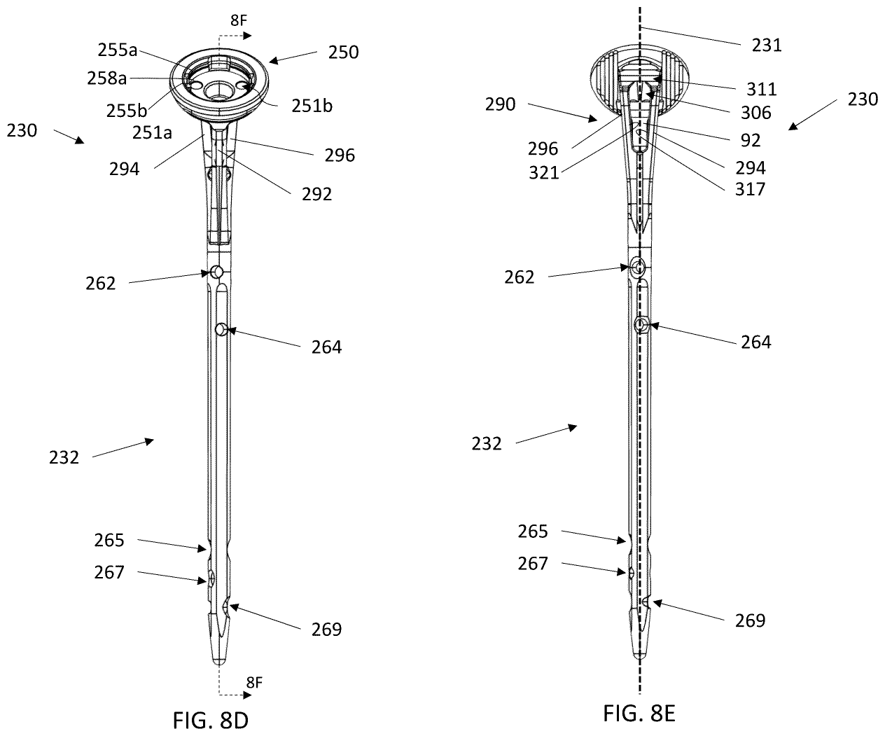

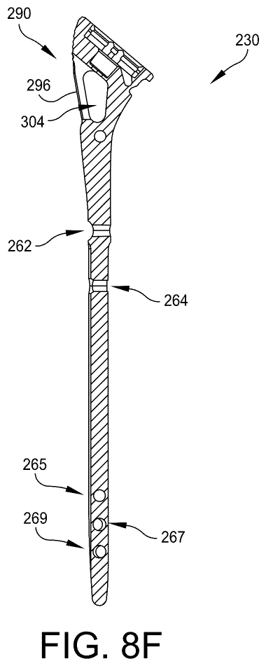

FIGS. 5A-7B illustrate an embodiment of a fracture stem 30. The stem 30 of FIGS. 1A and 2A is a fracture stem configured to be used in humeral fracture repair procedures as described herein. The stem 30 is configured to be anchored in a medullary canal of a humerus of a patient. As shown in FIG. 5A, the stem 30 includes a distal portion 32, a proximal portion 34, a medial side 93 and a lateral side 91. In some embodiments, the stem 30 is a unitary body. Accordingly, the stem 30 can be monolithic, and the distal portion 32 and proximal portion 34 can be integrally formed. In some embodiments, the stem 30 and/or other humeral anchors herein can have a distal portion that includes a taper. For example, the distal portion 32 can have a gradually tapered overall shape to better fit the humerus bone into which it is implanted. A length of the distal portion 32 of the stem 30 can vary, as further described below in relation to FIGS. 8A-8F.

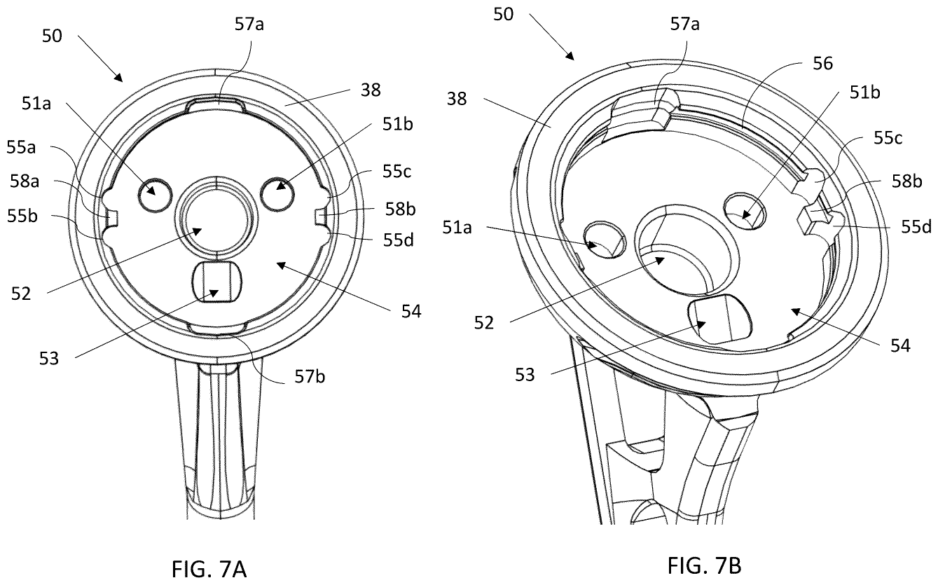

In some embodiments, the proximal portion 34 includes a spherical portion. For example, as shown in FIG. 5C, the outer surface 35 of the proximal portion 34 can be shaped generally as a half-sphere. A proximal end of the proximal portion 34 can include a stem face 50, which is further described below in relation to FIG. 7. The stem 30 can further include a metaphyseal portion 90 between the distal portion 32 and the proximal portion 34, as shown in FIGS. 5A and 5C. The metaphyseal portion 90 can include three or more arms extending between and connecting the distal portion 32 and the proximal portion 34. In the illustrated embodiment, the metaphyseal portion 90 includes three arms: a medial arm 92, a first lateral arm 94, and a second lateral arm 96. The medial arm 92 can be positioned near the calcar. The medial arm 92 can be angled medially. Accordingly, a proximal end 110 of the medial arm 92 can be positioned medially relative to a distal end 112 of the medial arm 92 as shown in FIG. 5C. For example, the proximal end 110 can be angled between about 10° and about 15°, or about 12° medially relative to the distal end 112. The first lateral arm 94 and second later arm 96 can be configured and positioned to support the tuberosities. The first lateral arm 94 and second lateral arm 96 can be angled outwardly or laterally. Accordingly, a proximal end 114 of the first lateral arm 94 can be positioned laterally relative to a distal end 116 of the first lateral arm 94, and a proximal end 118 of the second lateral arm 96 can be positioned laterally relative to a distal end 120 of the second lateral arm 96 as shown in FIG. 5C. The angle and shape of each arm 94, 96 can be selected based on virtual surgery and/or a numerical simulation of bone strain due to the prosthesis to adapt to the particular patient bony anatomy. For example, the proximal ends 114, 118 of the first and second lateral arms 94, 96 can be angled between about 5° and about 10°, or about 8° laterally relative to the distal ends 116, 120 of the first and second lateral arms 94, 96. The use of the fracture stem 30 of the present disclosure in a fracture repair procedure advantageously helps promote tuberosity healing and inhibit or reduce tuberosity resorption.

As shown in FIGS. 5B and 5C, the stem 30 can include a notch 108. The notch 108 can be at or near a location the medial side 93 of the metaphyseal portion 90 meets the proximal portion 34. For example, the notch 108 can be at or near a location where the medial side 93 of the medial arm 92 meets the proximal portion 34. The notch 108 can extend from this location along the medial side 93 of the proximal portion 34 to a peripheral rim 38 of the proximal portion 34. For example, a height H₁ of the notch 108 can be between about 5 mm and about 10 mm, between about 7 mm and about 8 mm, or about 8.6 mm. In some configurations, the height H₁ of the notch 108 can be between about 20% and about 50% or about 30% and about 40% of a height of the metaphyseal portion 90. The notch 108 can also extend along the outer surface 35 of the medial side 93 of the proximal portion 34 in an anterior to posterior direction. For example, the notch 108 can extend along at least a portion of a circumference of the proximal portion 34. In some configurations, the notch 108 only extends along the medial side of the proximal portion 34. In some configurations, the notch 108 can include a convex portion 108b between two concave portions 108a, 108c. The notch 108 can be configured to engage a suture in a fracture repair procedure and can help inhibit the suture from sliding or slipping out of position.

The stem 30 can also include a fin 102 protruding from the lateral side 91 of the distal portion 32. In the illustrated embodiment, the fin 102 extends from a proximal portion of the distal portion 32 distally along a portion of a length of the distal portion 32. The fin 102 can help promote correct positioning of the stem 30 during stem placement. In some configurations, a length of the fin 102 can be between about 20 mm and about 40 mm, between about 25 mm and about 35 mm, or about 30.8 mm. In some configurations, the length of the fin 102 can be between about 10% and about 40% or about 20% and about 30% of a total length L_{T1} (shown in FIG. 5F) of the stem 30.

In some configurations, a fenestration or window 104 can be defined between a lateral edge 113 of the medial arm 92 and medial edges 115, 119 of the first 94 and second 96 lateral arms, respectively. In some embodiments of a fracture repair procedure, a bone graft can be placed in the fenestration 104 to help promote bone-to-stem fixation. A space or gap 106, shown in FIGS. 5E, can be defined or formed between an inner edge 117 of the first lateral arm 94 and an inner edge 121 of the second lateral arm 96. In the illustrated embodiment, the gap 106 has an elongated, rounded rectangular shape, although other shapes or configurations are also possible. As shown, the gap 106 can extend from the proximal portion of the distal portion 32 to the proximal portion 34. The space 106 may enable increased bone growth and fixation, for example enabling bone graft materials to be placed within the fenestration 104, within the space 106, and/or on a lateral side of the lateral arms 94, 96. For example, in some procedures, the stem 30 can be used in a tuberosity fixation procedure using a horseshoe graft. An example of such a procedure is described in Levy, Jonathan C. and Badman, Brian, Reverse Shoulder Prosthesis for Acute Four-Part Fracture: Tuberosity Fixation Using a Horseshoe Graft, J Orthop Trauma, Volume 25, Number 5, May 2011. In such a procedure, the horseshoe graft can be placed on the lateral surface of the metaphyseal portion 90. The design of the metaphyseal portion can help provide stability to the horseshoe graft. The space 106 can allow the horseshoe graft to form or grow into or through the window 106 and improve fixation and tuberosity repair. In particular, resorption of the tuberosities can be reduced or prevented because more pathways for formation or growth of bone are provided to bridge between the fractured portions. The peripheral rim 38 of the stem face 50 can also help support and stabilize the tuberosities. The shape and size of the horseshoe graft can be selected based on a numerical simulation to accurately restore the tuberosities positions using virtual surgery.

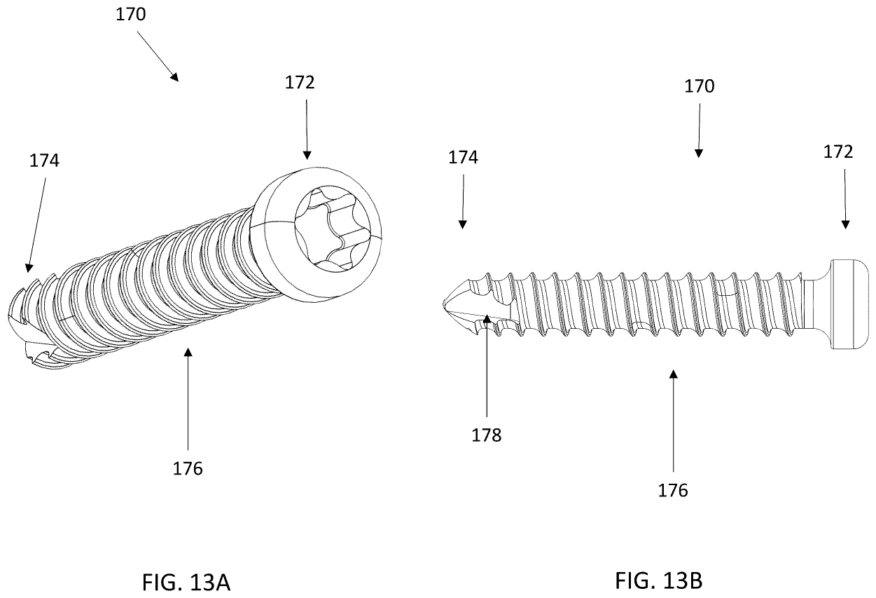

The metaphyseal portion 90 can include one or more through holes 98. For example, the one or more through holes 98 may be positioned below the fenestration 104. The through holes 98 can be configured to receive one or more screws 170 or plugs 700A, 700B, which are further described below in relation to FIGS. 13-16. The illustrated configuration shows only a single through hole 98 extending in the anterior-posterior direction. Advantageously, inserting a screw into the through hole 98 when implanting the stem 30 into the patient can improve stability and the mechanical strength of the stem 30. For example, using this additional screw can decrease the stress exerted on a weak point of the stem 30. The weak point may be located near or along the through hole 98 and the additional screw can decrease the stress exerted on this point by between about 30% and about 50%, or about 40% compared to the stress exerted on this point without the additional screw inserted in the through hole 98.

In some configurations, the distal portion 32 can include a plurality of grooves 130 extending in a longitudinal direction and are circumferentially spaced apart. For example, the distal portion 32 can have only four grooves 130. Each of the four grooves 130 can have a narrow distal end and a wider proximal end. In some configurations, the distal portion 32 of the stem 30 can include one or more apertures 62, 64 configured to receive one or more screws 170 or plugs 700A, 700B, which are further described below in relation to FIGS. 13-16. In the illustrated configuration, the distal portion 32 of the stem 30 includes only two apertures: a proximal aperture 62 and a distal aperture 64. In some configurations, at least one of the apertures 62, 64 can extend through at least one of the longitudinal grooves 130. In some configurations, each aperture 62, 64 may have a diameter of between about 2 mm and about 10 mm, about 4 mm and about 8 mm, or about 4.4 mm. In some configurations, each aperture 62, 64 may have a length of between about 2 mm and about 10 mm, about 4 mm and about 8 mm, or about 5.4 mm.

In some configurations, the apertures 62, 64 may be spaced apart from one another. For example, a distance between the two apertures 62, 64 can be between about 10 mm and about 30 mm, or about 15 mm and about 25 mm. In some configurations, the distance between the two apertures 62, 64 can be between about 10% and about 40% of a length of the distal shaft portion 32 or between about 20% and about 30% of the length of the distal shaft portion 32. In some configurations, the distal aperture 64 can be positioned between about 25 mm and about 45 mm or about 30 mm and about 40 mm from a distal tip 33 of the stem 30. In some configurations, the distance between the distal aperture 64 and the distal tip 33 can be between about 20% and about 50% of the length of the distal shaft portion 32, or about 30% and about 40% of the length of the distal shaft portion 32. In some configurations, the proximal aperture 62 can be positioned between about 45 mm and about 65 mm or about 50 mm and about 60 mm from the distal tip 33 of the stem 30. In some configurations, the distance between the proximal aperture 62 and the distal tip 33 can be between about 40% and about 70% of the length of the distal shaft portion 32, or about 50% and about 60% of the length of the distal shaft portion 32.

As shown in FIG. 5E, the aperture 62, 64 may be angled relative to a longitudinal plane 31 of the stem 30 such that a longitudinal centerline that extends along the length of each aperture 62, 64 is angled from the longitudinal plane 31. The longitudinal plane 31 can extend in a medial-lateral direction of the stem 30. In some configurations, the angle between the longitudinal centerline of the proximal aperture 62 and the longitudinal plane 31 may be between about 15° and about 75°, between about 30° and about 60°, or about 30°. In some configurations, the angle between the longitudinal centerline of the distal aperture 64 and the longitudinal plane 31 may be between about 15° and about 75°, between about 30° and about 60°, or about 30°. In some configurations, the proximal aperture 62 can be angled in the opposite direction from the distal aperture 64.

As shown in FIG. 5E, in some configurations, the lateral side 91 of the outer surface 35 of the proximal portion 34 may include a plurality of horizontal grooves 111. The plurality of horizontal grooves 111 can be configured to increase tuberosities stability. In some configurations, each of the lateral arms 94, 98 can include one or more horizontal grooves 111. In some configurations, the horizontal grooves 111 can be displaced from the proximal ends 114, 118 of the lateral arms 94, 98. For example, an area between the horizontal grooves 111 and the lateral arms 95, 98 may include a smooth surface. A maximum length of the plurality of the horizontal grooves 111 can be between about 10 mm and about 20 mm, about 12 mm and about 18 mm, or about 14 mm and about 16 mm. A minimum length of the horizontal grooves 111 can be between about 1 mm and about 10 mm, about 3 mm and about 8 mm, or about 5 mm and about 6 mm.

FIGS. 5F and 5G illustrate different dimensions of the stem 30. For example, the stem 30 can have a total length L_{T1} of between about 100 mm and about 150 mm, about 110 mm and about 140 mm, about 120 mm and about 130 mm, or about 136 mm. As shown in the illustrated configurations, the stem 30 may have varying widths. For example, the stem 30 may have a maximum width W_{T}, a first width W₁ measured in the medial-lateral direction at a first length L₁ from a distal tip 33 of the stem 30, a second width W₂ measured in the medial-lateral direction at a second length L₂ from the distal tip 33, and a third width W₃ measured in the medial-lateral direction and a third length L₃ from the distal tip 33. Additionally, the stem 30 may have a fourth width W₄ measured in the anterior-posterior direction at the first length L₁ from the distal tip 33, a fifth width W₅ measured in the anterior-posterior direction at the third length L₃ from the distal tip 33, and a sixth width W₆ measured in the anterior-posterior direction at a fourth length L₄ from the distal tip 33.

In some configurations, the first length L₁ can be between about 30 mm and about 70 mm, about 40 mm and about 60 mm, or about 59 mm. In some configurations, the first length L₁ can be between about 30% and about 60% of the total length L_{T1}, about 40% and about 50% of the total length L_{T1}, or about 43% of the total length L_{T1}. In some configurations, the second length L₂ can be between about 60 mm and about 100 mm, about mm and about 90 mm, or about 80 mm. In some configurations, the second length L₂ can be between about 40% and about 70% of the total length L_{T1}, about 50% and about 60% of the total length L_{T1}, or about 59% of the total length L_{T1}. In some configurations, the third length L₃ can be between about 80 mm and about 120 mm, about 90 mm and about 110 mm, or about 96 mm. In some configurations, the third length L₃ can be between about 50% and about 80% of the total length L_{T1}, about 60% and about 70% of the total length L_{T1}, or about 71% of the total length L_{T1}. In some configurations, the fourth length L₄ can be between about 90 mm and about 130 mm, about 100 mm and about 120 mm, or about 107 mm. In some configurations, the fourth length L₄ can be between about 60% and about 90% of the total length L_{T1}, about 70% and about 80% of the total length L_{T1}, or about 79% of the total length L_{T1}.

In some configurations, the maximum width W_{T} can be less than the total length L_{T1}. For example, the maximum width W_{T} can be between about 20 mm and about mm, about 30 mm and about 40 mm, or about 38 mm. In some configurations, the maximum width W_{T} can be between about 10% and about 40% of the total length L_{T1}, about 20% and about 30% of the total length L_{T1}, or about 28% of the total length L_{T1}. In some configurations, the first width W₁ can be between about 5 mm and about 20 mm, or about 10 mm and about 15 mm. In some configurations, the first width W₁ can be between about 10% and about 60% of the maximum width W_{T}, about 20% and about 50% of the maximum width W_{T}, or about 30% and about 40% of the maximum width W_{T}. In some configurations, the second width W₂ can be between about 7 mm and about 25 mm, or about 10 mm and about 20 mm. In some configurations, the second width W₂ can be between about 10% and about 70% of the maximum width W_{T}, about 20% and about 60% of the maximum width W_{T}, or about 30% and about 50% of the maximum width W_{T}. In some configurations, the third width W₃ can be between about 10 mm and about 25 mm, or about 15 mm and about 20 mm. In some configurations, the third width W₃ can be between about 20% and about 70% of the maximum width W_{T}, about 30% and about 60% of the maximum width W_{T}, or about 40% and about 50% of the maximum width W_{T}. In some configurations, the fourth width W₄ can be between about 5 mm and about 20 mm, or about 10 mm and about 15 mm. In some configurations, the fourth width W₄ can be between about 10% and about 60% of the maximum width W_{T}, about 20% and about 50% of the maximum width W_{T}, or about 30% and about 40% of the maximum width W_{T}. In some configurations, the fifth width W₅ can be between about 8 mm and about 20 mm, or about 10 mm and about 15 mm. In some configurations, the fifth width W₅ can be between about 10% and about 60% of the maximum width W_{T}, about 20% and about 50% of the maximum width W_{T}, or about 30% and about 40% of the maximum width W_{T}. In some configurations, the sixth width W₆ can be between about 10 mm and about 20 mm, or about mm and about 15 mm. In some configurations, the first width W₁ can be between about 20% and about 70% of the maximum width W_{T}, about 30% and about 60% of the maximum width W_{T}, or about 40% and about 50% of the maximum width W_{T}. The table below provides example widths for different sizes of the stem 30.

| | W₁ | W₂ | W₃ | W₄ | W₅ | W₆ |
|---|---|---|---|---|---|---|
| Size | (mm) | (mm) | (mm) | (mm) | (mm) | (mm) |
| Size 1 | 6.6 | 8.7 | 12.2 | 6.6 | 9.1 | 11.3 |
| Size 2 | 8.0 | 9.5 | 12.5 | 8.0 | 10.2 | 12.0 |
| Size 3 | 8.6 | 10.0 | 12.6 | 8.6 | 12.3 | 13.7 |
| Size 4 | 8.6 | 10.5 | 14.1 | 8.6 | 12.3 | 13.7 |
| Size 5 | 9.6 | 11.4 | 14.6 | 9.6 | 15.4 | 16.2 |
| Size 6 | 10.6 | 12.2 | 15.2 | 10.6 | 15.6 | 16.2 |
| Size 7 | 11.6 | 13.4 | 15.8 | 11.6 | 15.7 | 16.2 |
| Size 8 | 12.6 | 14.6 | 16.5 | 12.6 | 15.8 | 16.2 |
| Size 9 | 13.6 | 15.5 | 17.3 | 13.6 | 15.9 | 16.2 |
| Size 10 | 14.6 | 16.3 | 18.0 | 14.6 | 16.0 | 16.2 |
| Size 11 | 15.6 | 17.8 | 19.8 | 15.6 | 17.0 | 17.2 |
| Size 12 | 16.6 | 19.2 | 21.5 | 16.6 | 18.0 | 18.2 |

FIGS. 7A and 7B illustrates the stem face 50 of the stem 30. As previously described, the stem face 50 can include a first recess 52 recessed from a distal end of a second recess 54. As shown in FIGS. 7A-7B, for example, the second recess 54 may be wider and larger than the first recess 52. In some embodiments, the second recess 54 may be defined by generally cylindrical or only slightly tapered walls. Similarly, the first recess 52 may be defined by generally cylindrical or only slightly tapered walls. In some embodiments, the second recess 54 can be sized and shaped to receive an insert 161. For example, the first recess 52 can be sized and shaped to receive a portion of the coupler 168 to convert the reverse anatomical reconstruction device of FIG. 4 to the anatomical reconstruction device of FIG. 1A. Moreover, the first recess 52 can be sized and shaped to receive an engagement feature 156A, 156B, 156C of the one or more spacers and adapters 150.

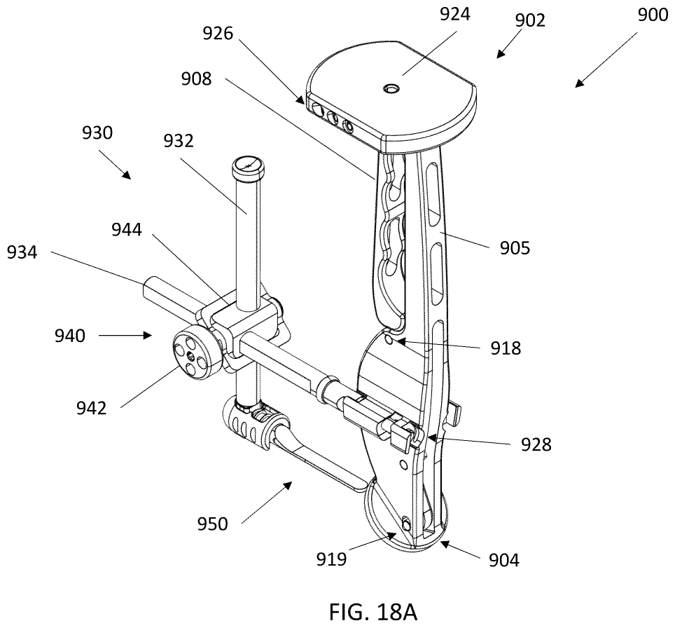

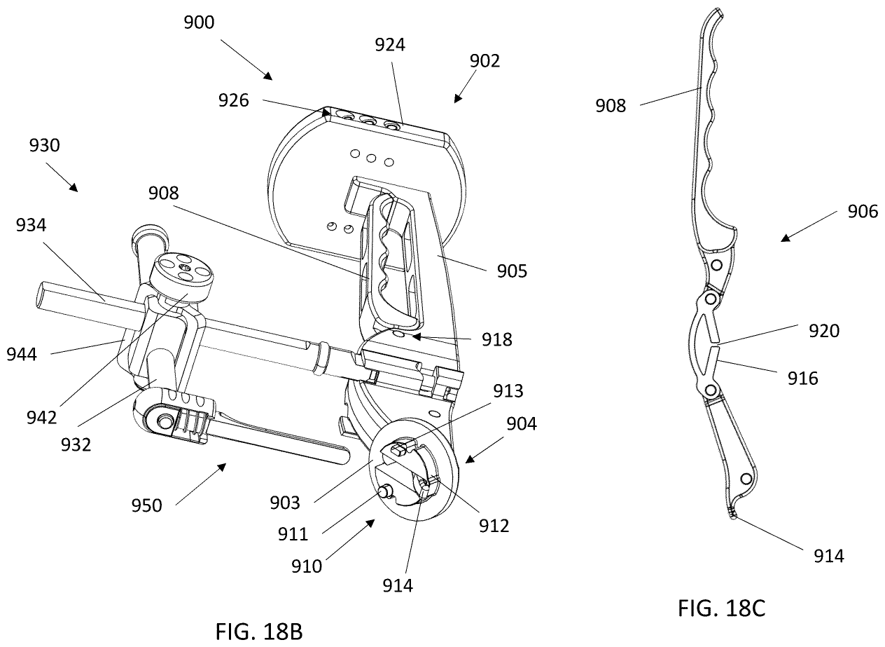

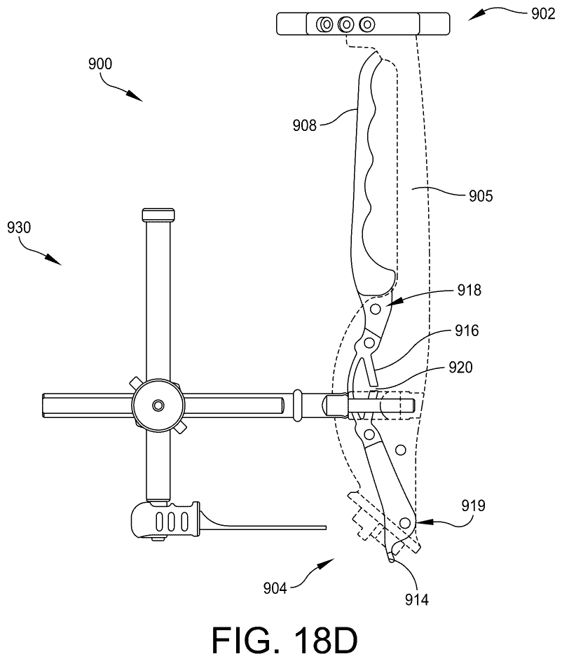

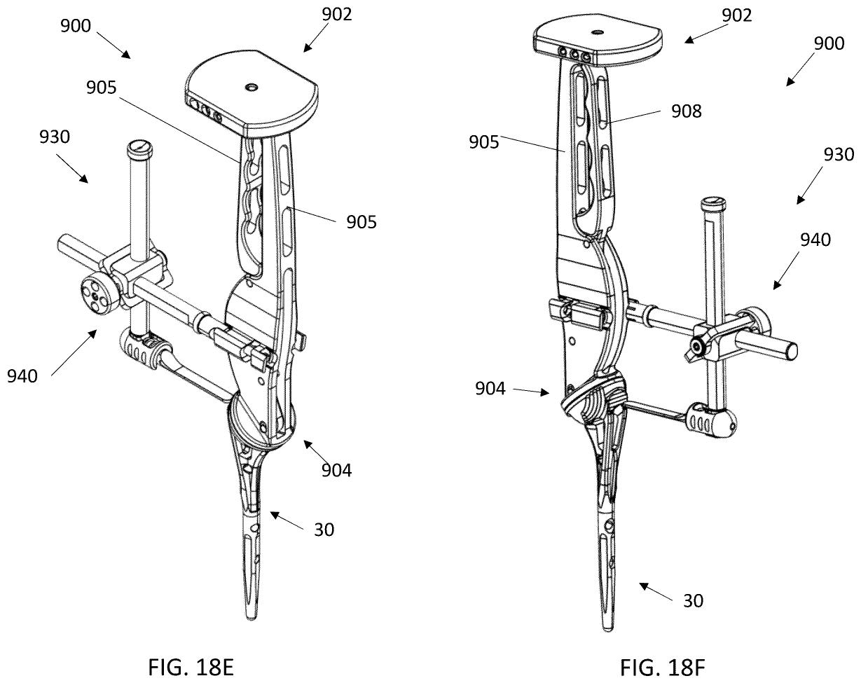

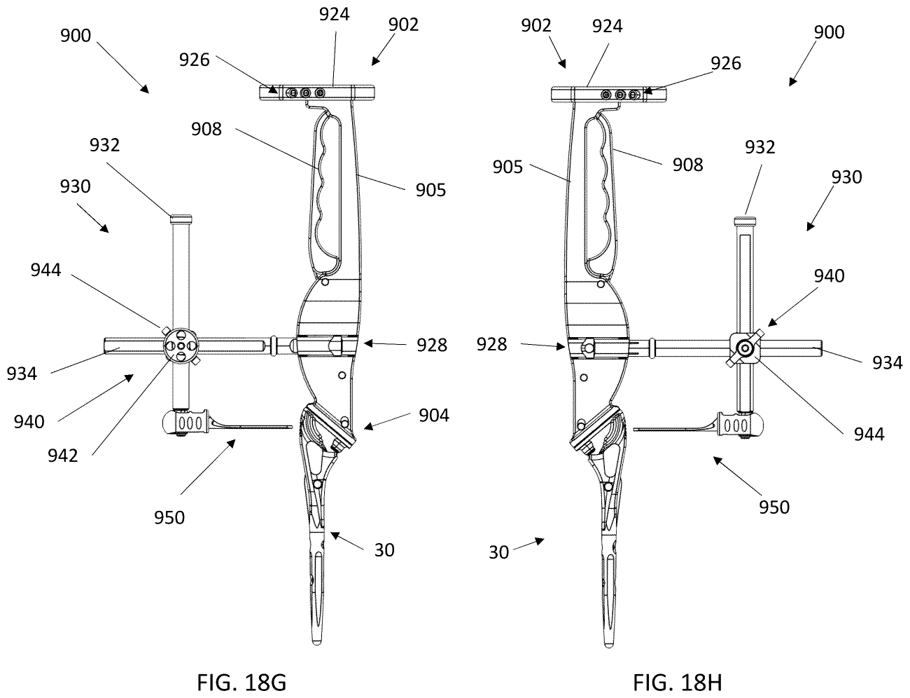

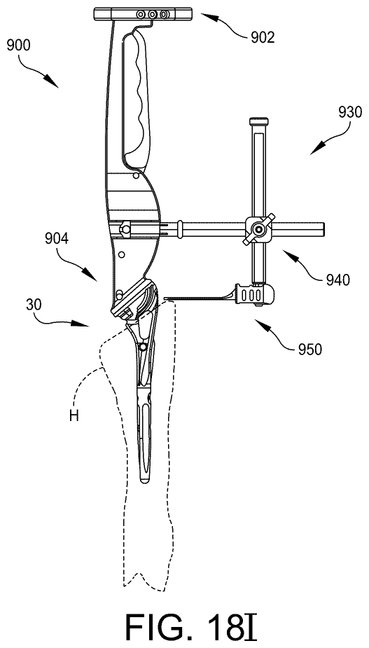

As shown in FIGS. 7A and 7B, the stem face 50 can comprise one or more interfacing components such as one or more apertures 51a, 51b, 53, a groove 56, and one or more slots 55a, 55b, 55c, 55d, 57a, 57b. The one or more apertures 51a, 51b, 53 can include first and second aperture 51a, 51b and an anti-rotation aperture 53. In some configurations the one or more apertures 51a, 51b, 53 can be configured to engage with certain portions of the anatomic articular component 160, the reverse articular component 180, the one or more spacers 150, and/or a tool configured to insert the stem 30 into the bone of patient (e.g., a stem holder 900 or a jig 1000). For example, the anti-rotation aperture 53 may be configured to engage with a protrusion 155A, 155B of the one or more spacers and adapters 150. When assembled, the protrusion 155A, 155B can extend into the anti-rotation aperture 53 to minimize or eliminate rotation of the spacer or adapter 150 in relation to the stem face 50 of the stem 30. In some configurations, the one or more apertures 51a, 51b, 53 can be configured to engage with a tool, such as the stem holder 900 or the jig 1000, which are further described below in relation to FIGS. 18A-21E.

In some configurations, the one or more slots 55a, 55b, 55c, 55d, 57a, 57b can be sized and configured to engage an insert 161 (such as the articular components 160, 180). For example, the slots 55a, 55b, 55c, 55d, 57a, 57b can engage or receive corresponding ridges of an insert 161. As explained herein, the slots 55a, 55b, 55c, 55d, 57a, 57b can limit rotation of the insert 161 relative to the anchor 30. The slots 55a, 55b, 55d, 57a, 57b can also guide the advancement of the insert 161 into an upper portion of the second recess 54. The slots 55a, 55b, 55c, 55d, 57a, 57b can be disposed vertically along the stem face 50 and can be circumferentially spaced from one another. For example, first and second slots 55a, 55b may be positioned adjacent each other and opposite third and fourth slots 55c, 55d. Further, fifth and sixth slots 57a, 57b may be positioned opposite one another. In some configurations, the fifth and sixth slots 57a, 57b may have a greater width than the first, second, third, and fourth slots 55a, 55b, 55c, 55d. In the embodiment of FIGS. 7A and 7B, the slots 55a, 55b, 55c, 55d, 57a, 57b can extend from a location proximate the peripheral rim 38 towards the bottom of the second recess 54. In some configurations, the stem face 50 may include one or more protrusions 58a, 58b between adjacent slots 55a, 55b, 55c, 55d. For example, the one or more protrusions 58a, 58b may include a first protrusion 58a between adjacent slots 55a, 55b and a second protrusion 58b between adjacent slots 55c, 55d. The first and second protrusions 58a, 58b may be configured to engage with a portion of the insert 161 (e.g., a distal portion 152A, 152B, 152C of the one or more spacer and adapter 150). Further, the stem face 50 can include the groove 56 extending circumferentially about the second recess 54. The groove 56 can be sized and configured to receive a locking ring of an articular body assembly (e.g., any of the inserts 161 described herein).

FIGS. 8A-8F illustrate another embodiment of the stem 230 similar to the embodiments of the stem 20 illustrated in and described in relation to FIGS. 3-7B. Reference numerals of the same or substantially the same features may share the same last two digits. As shown in FIG. 8B, the stem 230 may have a total length L_{T2} greater than the total length L_{T1} of the stem 30. For example, the total length L_{T2} of the stem 230 can be between about 150 mm and about 250 mm, about 160 mm and about 240 mm, about 170 mm and about 230 mm, or about 215 mm. In some configurations, the total length L_{T2} of the longer stem 230 can be between about 130% and about 180%, about 140% and about 170%, about 150% and about 160%, or about 158% of the total length L_{T1} of the shorter stem 30.

As shown in FIGS. 8C-8F, the distal portion 232 of the stem 230 can comprise one or more apertures 262, 264, 265, 267, 269 configured to receive one or more screws 170 or plugs 700A, 700B, which are further described below in relation to FIGS. 13-16. In the illustrated configuration, the distal portion 232 of the stem 230 can include only five apertures: a first aperture 262, a second aperture 264, a third aperture 265, a fourth aperture 267, and a fifth aperture 269. The first and second apertures 262, 264 can be the same as or similar to the proximal and distal apertures 62, 64 of the stem 30. For example, as shown in FIG. 8E, the first and second apertures 262, 264 may be angled relative to a longitudinal plane 231 of the stem 230 such that a longitudinal centerline that extends along the length of each aperture 262, 264 is angled from the longitudinal plane 231. The longitudinal plane 231 can extend in a medial-lateral direction of the stem 230. In some configurations, the angle between the longitudinal centerline of the first aperture 262 and the longitudinal plane 231 may be between about 15° and about 75°, between about 30° and about 60°, or about 30°. In some configurations, the angle between the longitudinal centerline of the second aperture 264 and the longitudinal plane 231 may be between about and about 75°, between about 30° and about 60°, or about 30°. In some configurations, the first aperture 262 can be angled in the opposite direction from the second aperture 264.

As shown in FIG. 8E, the third, fourth, and fifth apertures 265, 267, 269 may be angled relative to a second longitudinal plane (not shown) that is substantially normal to the longitudinal plane 231. For example, the longitudinal centerline of the third aperture 265 may extend in the same direction as the second longitudinal plane such that the third aperture 265 extends in an anterior-posterior direction. In some configurations, the angle between the longitudinal centerline of the fourth aperture 267 and the second longitudinal plane may be between about 10° and about 30°, between about 15° and about or about 20°. In some configurations, the angle between the longitudinal centerline of the fifth aperture 269 and the second longitudinal plane may be between about 10° and about 30°, between about 15° and about 25°, or about 20°. In some configurations, the fourth aperture 267 can be angled in the opposite direction from the fifth aperture 269.

In some configurations, the first and second apertures 262, 264 may be spaced apart from one another. For example, the distance between the two apertures 62, 64 can be between about 4% and about 20% of the total length L_{T2} of the stem 230 or about 10% and about 15% of the total length L_{T2} of the stem 230. In some configurations, the first aperture 262 can be positioned between about 130 mm and about 160 mm or about 140 mm and about 150 mm from a distal tip 233 of the stem 230. In some configurations, the distance between the first aperture 262 and the distal tip 233 can be between about 45% and about 75% of the total length L_{T2} of the stem 230, or about 55% and about 65% of the total length L_{T2} of the stem 230. In some configurations, the second aperture 262 can be positioned between about 105 mm and about 135 mm or about 115 mm and about 125 mm from the distal tip 233 of the stem 230. In some configurations, the distance between the second aperture 264 and the distal tip 233 can be between about 40% and about 80% of the total length L_{T2} of the longer stem 230, or about 50% and about 70% of the total length L_{T2} of the longer stem 230.

In some configurations, the first and second apertures 262, 264 may be spaced apart from the third, fourth, and fifth apertures 265, 267, 269. For example, a distance between the second aperture 264 and the third aperture 265 can be between about mm and about 100 mm, or about 70 mm and about 90 mm. In some configurations, the distance between the second aperture 264 and the third aperture 265 can be between about 20% and about 60% of the total length L_{T2} of the stem 230 or about 30% and about 50% of the total length L_{T2} of the stem 230.

In some configurations, the third aperture 265 can be positioned between about 20 mm and about 60 mm or about 30 mm and about 50 mm from a distal tip 233 of the stem 230. In some configurations, the distance between the third aperture 265 and the distal tip 233 can be between about 5% and about 40% of the total length L_{T2} of the stem 230, or about 10% and about 30% of the total length L_{T2} of the stem 230. In some configurations, the fourth aperture 267 can be positioned between about 10 mm and about mm or about 20 mm and about 40 mm from the distal tip 233 of the stem 230. In some configurations, the distance between the fourth aperture 267 and the distal tip 233 can be between about 5% and about 30% of the total length L_{T2} of the longer stem 230, or about 10% and about 20% of the total length L_{T2} of the longer stem 230. In some configurations, the fifth aperture 269 can be positioned between about 5 mm and about 40 mm or about 10 mm and about 30 mm from the distal tip 233 of the stem 230. In some configurations, the distance between the fifth aperture 269 and the distal tip 233 can be between about 5% and about 30% of the total length L_{T2} of the longer stem 230, or about 10% and about 20% of the total length L_{T2} of the longer stem 230.

In some configurations, the third aperture 265 can be configured to receive a screw 170 when securing the stem 230 in a left or right shoulder of a patient. In some configurations, the fourth aperture 257 can be configured to receive the screw 170 when securing the stem 230 in the right shoulder of the patient. In some configurations, the fifth aperture 259 can be configured to receive the screw 170 when securing the stem 230 in the left shoulder of the patient. In other configurations the fourth aperture 257 can be configured to receive the screw 170 when securing the stem 230 in the left shoulder of the patient and the fifth aperture 259 can be configured to receive the screw 170 when securing the stem 230 in the left shoulder of the patient.

### III. Examples of Components of the Humeral Assembly

As explained above in relation to FIG. 3, the shoulder arthroplasty system or humeral assembly can include a number of components, such as an adapter 168, a spacer 150, a plurality of screws 170, and/or a plurality of plugs 700A, 700B.

FIGS. 9A-11C illustrate different embodiments of the spacer 150. FIGS. 9A-9C illustrate an embodiment of a spacer 150A that is asymmetric. The spacer 150A can include a proximal portion 151A and a distal portion 152A. The proximal portion 151A may have a diameter greater than the diameter of the distal portion 152A such that the proximal portion 151A extends radially outward from the distal portion 152A. In some configurations, the distal portion 152A extends from a distal facing surface 153A of the proximal portion 151A.

As shown in FIGS. 9A and 9B, the distal portion 152A can include a distal facing surface 154A. The distal facing surface 154A can include a protrusion 155A and an engagement feature 156A. In some configurations, the protrusion 155A and the engagement feature 156A can extend distally from the distal facing surface 154A. As described above, the protrusion 155A can be configured to engage with the anti-rotation aperture 53, 253 of the stem 30, 230. When the spacer 150A is coupled to the stem 30, 230, the protrusion 155A can extend into the anti-rotation aperture 53, 253 to minimize or eliminate rotation of the spacer 150A in relation to the stem face 50, 250 of the stem 30, 230. The engagement feature 156A can be configured to engage with the first recess 52, 252 of the stem face 50, 250 of the stem 30, 230. When the spacer 150A is coupled to the stem 30, 230, the engagement feature 156A can extend into the first recess 52, 252. In some configurations, the engagement feature 156A can have a substantially cylindrical shape or any other suitable shape. In some configurations, the engagement feature 156A can be positioned at or near the center of the distal facing surface 154A. In some configurations, the protrusion 155A may be positioned medially or laterally from the engagement feature 156A.

As shown in FIGS. 9A and 9B, the distal portion 152A can include a curved outer surface with one or more cutouts 157A. The one or more cutouts 157A can be positioned on opposite sides of the distal portion 152A. The one or more cutouts 157A can be configured to align with the one or more slots 55a, 55b, 55c, 55d, 255a, 255b, 255c, 255d and the first and second protrusions 58a, 58b, 258a, 258b of the stem face 50, 250. For example, the one or more cutouts 157A can engage with the one or more slots 55a, 55b, 55d, 255a, 255b, 255c, 255d and the first and second protrusions 58a, 58b, 258a, 258b to minimize or eliminate rotation of the spacer 150A in relation to the stem face 50, 250 of the stem 30, 230.

As shown in FIG. 9B, the proximal portion 151A of the asymmetric spacer 150A has a distal surface 1515 that is generally parallel to the stem face 50, 250 of the stem 230 that the spacer 150A is engaging with when the spacer 150A is used with the stem 230. When the spacer 150A is used with the stemless humeral anchor 103, the distal surface 1515 is generally parallel to the engaging face of the stemless humeral anchor 103. A longitudinal axis L of the spacer 150A is defined that is orthogonal to the distal surface 1515. The proximal portion 151A of the asymmetric spacer 150A is asymmetric with respect to the longitudinal axis L.

The asymmetry of the asymmetric spacer 150A is defined as follows. As shown in FIG. 9B, a distal edge 158A of the proximal portion 151A on a medial side 93A of the spacer 150A extends farther medially than a proximal edge 159A of the proximal portion 151A on the medial side 93A of the spacer 150A and the distal edge 158A on the lateral side 91A of the spacer 150A can extend medially relative to the proximal edge 159A on the lateral side 91A of the spacer 150A.

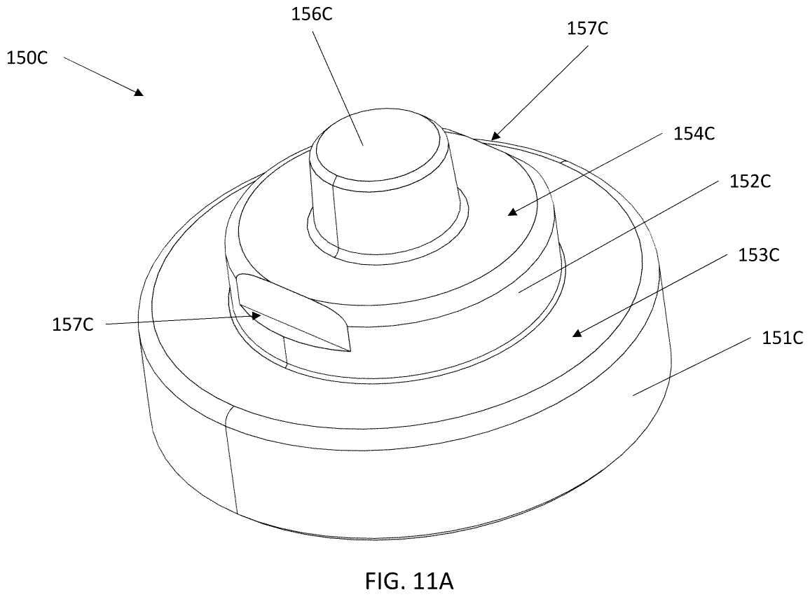

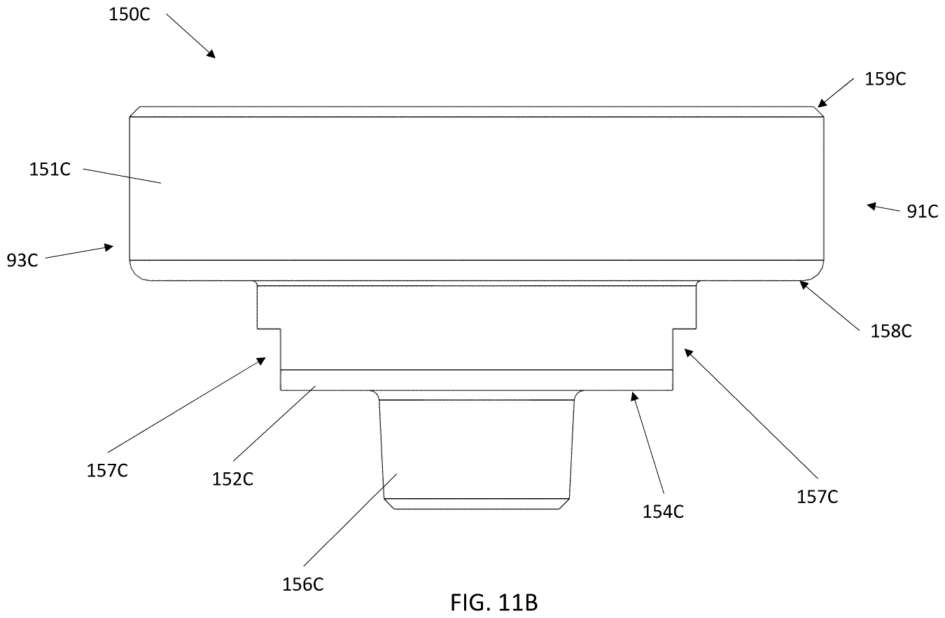

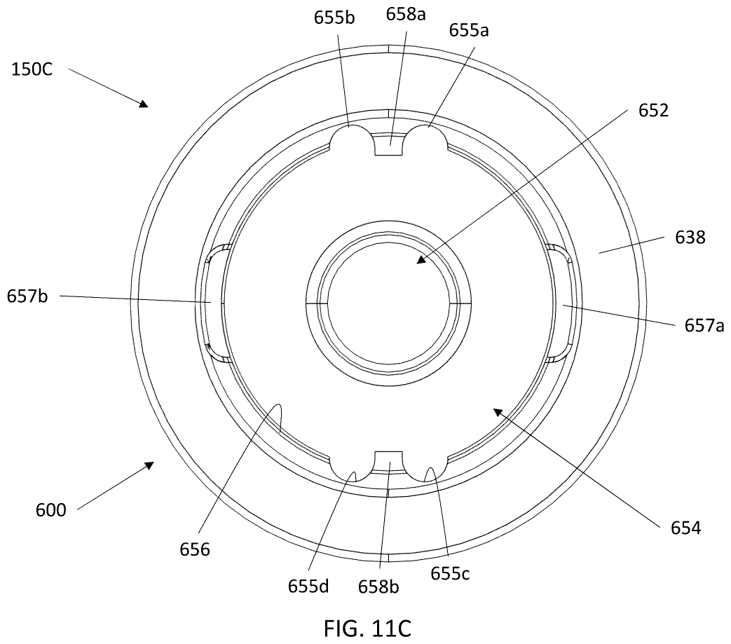

Compared to the symmetric cylindrical spacer 150C shown in FIGS. 11A-11C, the asymmetric configuration of the spacer 150A enables selective distalization of the humerus while minimizing lateralization of the humerus. In other words, the use of the asymmetric configuration of the spacer 150A selectively extends or lengthens the humerus by locating the articulating assembly of reverse prosthetic shoulder joint farther in the proximal direction away from the stem face 50, 250 at the proximal end of the humerus while minimizing the amount of lateralization of the humerus with respect to the shoulder joint. This feature can be useful in treating conditions where the pre-existing humeral stem was healed or cemented too low in the humerus, for example. In such situation, the asymmetric spacer 150A can be used to effectively increase the height of the humeral component of the prosthetic joint without revising the humeral stem. The asymmetric feature can also be useful in allowing tuberosity fixation using the fracture stems 30, 230 without or minimal lateralization.

This beneficial effect of the asymmetric spacer 150A is now described with reference to FIGS. 4B and 4C. FIG. 4B is an illustration of a reverse shoulder prosthesis assembly of a fracture stem 30, 230, a symmetric spacer 150C, a reverse articular component 180, 180A, with an articular body 164 (a glenosphere) engaging the reverse articular component 180, 180A. FIG. 4C for comparison is an illustration of a reverse shoulder prosthesis assembly shown in FIG. 4B except that the spacer 150A is an asymmetric spacer.

FIGS. 4B and 4C show that in the prosthetic assembly of FIG. 4C, the asymmetric shape of the asymmetric spacer 150A has shifted the whole prosthetic assembly generally in the superior direction. This is illustrated by the relative position of the center of the glenosphere 164 marked by “X” with respect to the most proximal point A of the stem 30, 230. In FIG. 4B, the center “X” of the glenosphere 164 is below the most proximal point A of the stem 30, 230 by a distance D. In FIG. 4C, the center “X” of the glenosphere 164 has been moved in the superior direction and is almost at the same level with the most proximal point A of the stem 30, 230. However, the overall width W′ of the prosthetic assembly is reduced because of the angle of the stem face 50, 250 of the stem 230. The width W′ of the asymmetric spacer 150A assembly in FIG. 4C is smaller than the width W of the symmetric spacer 150C assembly in FIG. 4C. The asymmetric spacer 150A can also be used in combination with the stemless humeral anchors 103 or humeral stem 113 in assembling a reverse prosthetic shoulder joint arrangement for the same beneficial reasoning described.

The stem implants 30 and 230 are examples of fracture stem implants. However, the asymmetric spacer 150A can also be used with standard humeral stem implants to achieve the similar beneficial effect of selectively distalizing the humerus while iminizing lateralization of the humerus.

As shown in FIG. 9C, the proximal portion 151A can include a proximal face 400. The proximal face 400 can be the same as or substantially similar to the stem face 50 of the stem 30 illustrated in and described in relation to FIGS. 7A and 7B. Reference numerals of the same or substantially the same features may share the same last two digits. For example, the proximal face 400 can be configured to couple the stem face 230 to the reverse articular component 180, the adapter or coupler 168, or the articular body 164. The proximal face 400 can include a first recess 452, a second recess 454, a peripheral rim 438, a groove 456, one or more slots 455a, 455b, 455c, 455d, 457a, 457b, and first and second protrusions 457a, 457b. As shown in the illustrated configurations, the proximal face 400 may include only the aforementioned features. In other configurations, the proximal face 400 can also include one or more apertures similar to or the same as the one or more apertures 51a, 51b, 53 of the stem 30.

With the asymmetric spacer 150A in the arthroplasty kit 100, a surgeon can use the asymmetric spacer 150A to adjust the position of the reverse shoulder prosthetic joint. In the case of an initial arthroplasty procedure assembling a reverse prosthetic shoulder joint, after a stemless humeral anchor 103 or a stemmed humeral anchor 113 is positioned in the proximal end of the prepared humerus, the reverse articular component 180 or 180A can be attached to the anchor 103, 113 with an asymmetric spacer 150A in between to position the articulating assembly (comprising the reverse articular component 180 or 180A and a glenosphere 164) to selectively distalize the humerus while minimizing any lateralization of the humerus. An asymmetric spacer 150A having the appropriate thickness and appropriate amount of asymmetry would be selected for a given patient. In the case of a revision arthroplasty, if the position of the originally placed stemless humeral anchor 103 or the originally placed stemmed humeral anchor 113 is too low in the humerus, an appropriately dimensioned asymmetric spacer 150A can be positioned between the humeral anchor 103 or 113 and the reverse articular component 180, 180A.

In some embodiments, a protrusion similar to the protrusion 155A can be placed at a different location on the distal facing surface 154A to function as a locating key for keying the asymmetric spacer to a different rotational position. This can be used to vary the direction of the distalization. In some embodiments, the protrusion 155A on the distal facing surface 154A of the spacer can be omitted to allow arbitrary rotation of the asymmetric spacer. Either way, some examples of benefits from having the ability to dial in the direction of the asymmetry are (but now limited to these): a) pure distalization would allow to tension more deltoid and get additional construct stability of the shoulder without adding tension on fractured tuberosity (in a fracture cases, surgeons generally do not want to tighten too much on the tuberosity to enhance healing without tuberosity migration); b) anterior or posterior offset to accommodate internal rotation/external rotation balance; c) anterior or posterior offset to accommodate bone distortion (Fracture sequalae for instance); d) anterior or posterior offset to accommodate joint subluxation in a tight shoulder. and e) optimizing impingement free range of motion by the ability to place the tray offset in any direction.

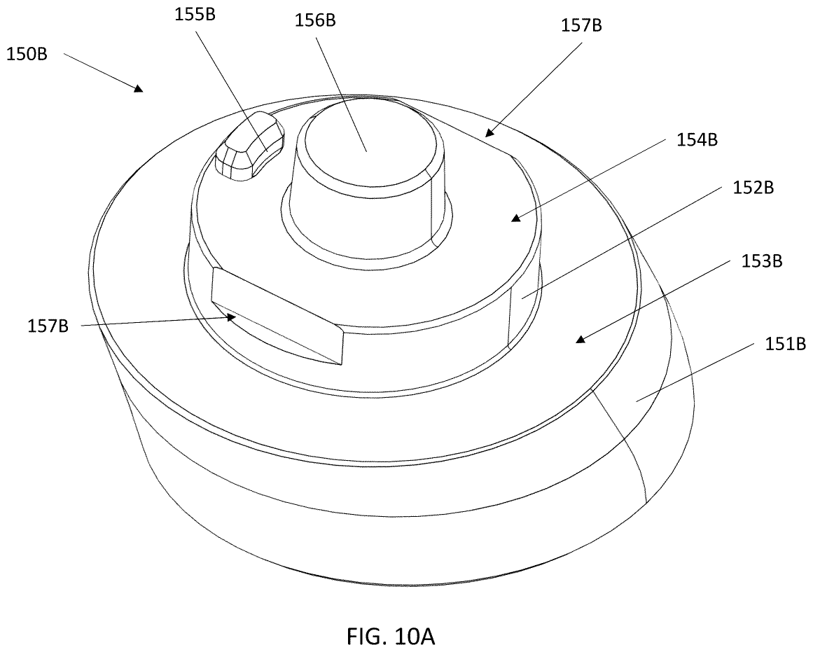

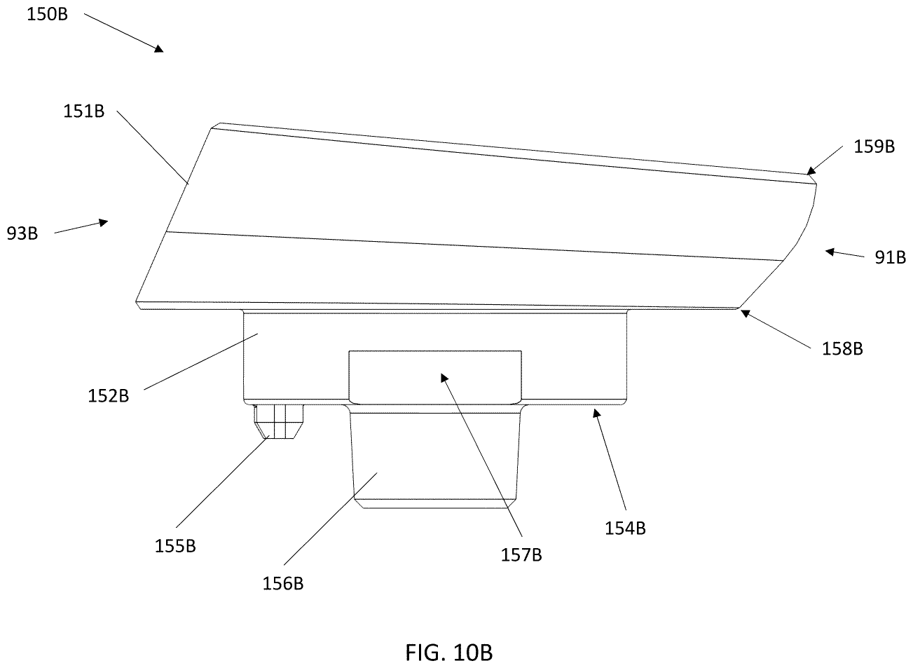

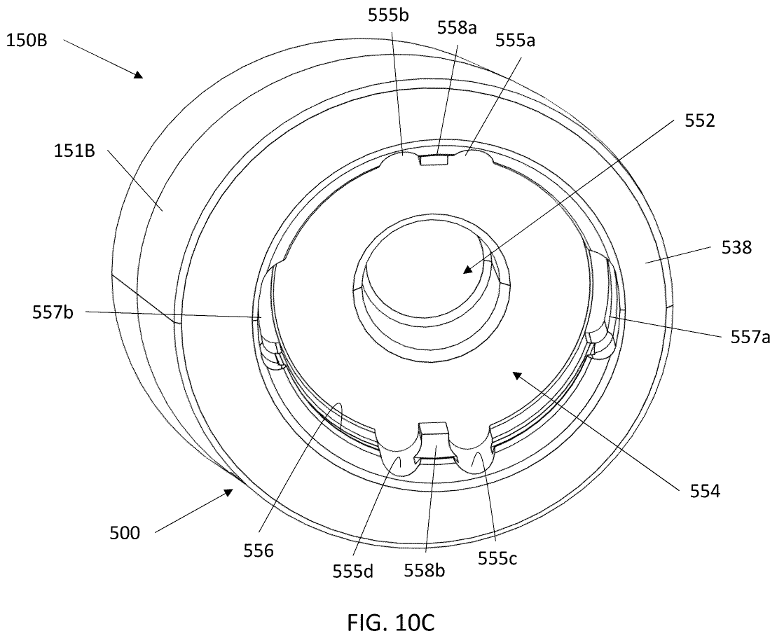

FIGS. 10A-10B illustrate another embodiment of the spacer 150B similar to the embodiments of the spacer 150A illustrated in and described in relation to FIGS. 9A-9B. Reference numerals of the same or substantially the same features may share the same first three digits. As shown in FIG. 10B, the proximal portion 151B may be angled. For example, the proximal edge 159B of the proximal portion 151B may be angled relative to the distal edge 158B of the proximal portion 151B such that the proximal edge 159B on the medial side 93B of the spacer 150B extends farther proximally than the proximal edge 159B on the lateral side 91B of the spacer 150B. For example, the proximal edge 159B can be angled between about 1° and about 20°, or about 5° and about 15° about 5° relative to the distal edge 158B. In some configurations, the proximal edge 159B on the medial side 93B of the spacer 150B can extend farther distally than the proximal edge 159B on the lateral side 91B of the spacer 150B.

FIG. 10C illustrates the proximal face 500 of the spacer 150B, which can be similar to the proximal face 400 of the spacer 150A and the stem face 50 of the stem 30 illustrated in and described in relation to FIGS. 7A-7B and 9C. Reference numerals of the same or substantially the same features may share the same last three digits.

FIGS. 11A-11B illustrate another embodiment of the spacer 150C similar to the embodiments of the spacer 150A illustrated in and described in relation to FIGS. 9A-9B. Reference numerals of the same or substantially the same features may share the same first three digits. As shown in FIGS. 11A and 11B, the distal portion 152C can include only the engagement feature 156C and the one or more cutouts 157C on the curved outer surface of the distal portion 152C. In some configurations, the distal portion 152C can include a protrusion similar to or the same as the protrusion 155A of the spacer 150C. As shown in FIG. 11B, the spacer 150C may be symmetrical about the longitudinal axis and/or non-angled. For example, the distal edge 158C of the proximal portion 151C on both sides 91C, 93C of the spacer 150C aligns with the proximal edge 159C of the proximal portion 151C on the both sides 91C, 93C of the spacer 150C.

FIG. 11C illustrates the proximal face 600 of the spacer 150B, which can be similar to the proximal face 400 of the spacer 150A and the stem face 50 of the stem 30 illustrated in and described in relation to FIGS. 7A-7B and 9C. Reference numerals of the same or substantially the same features may share the same last three digits.

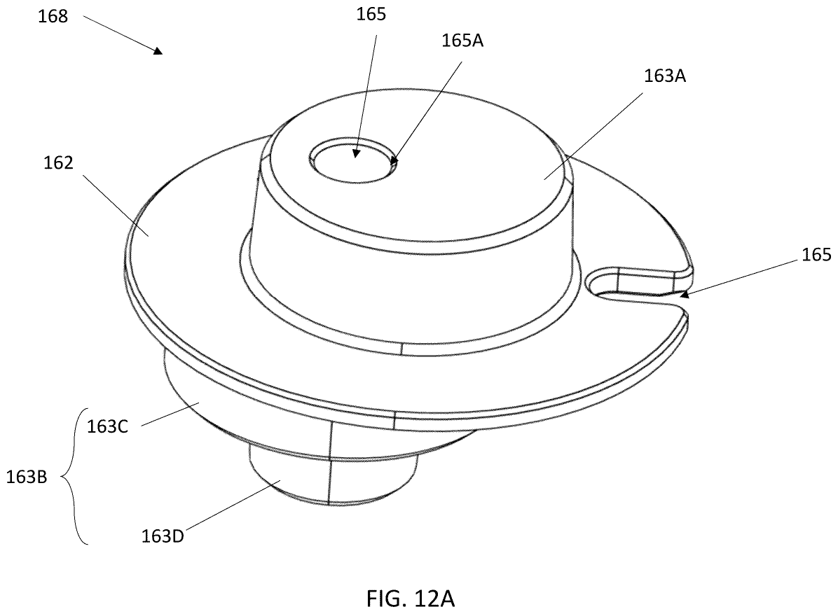

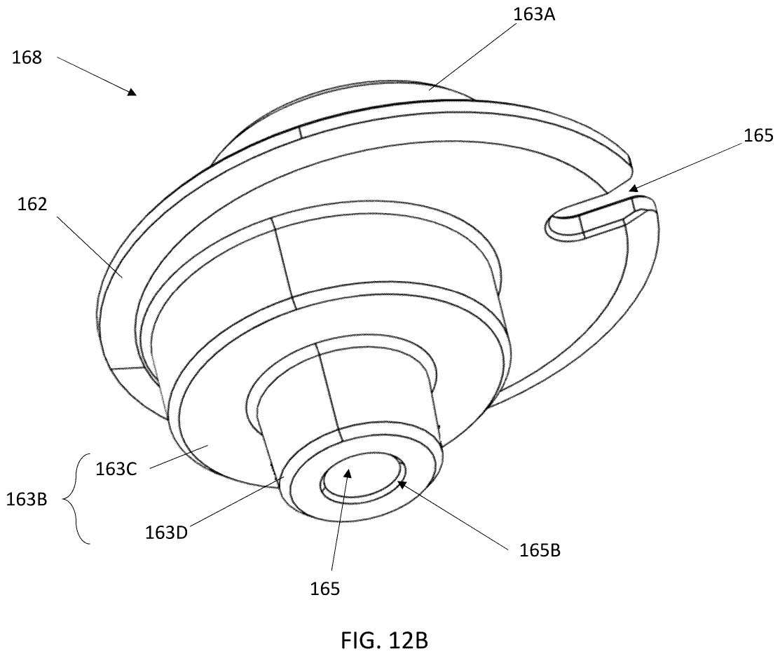

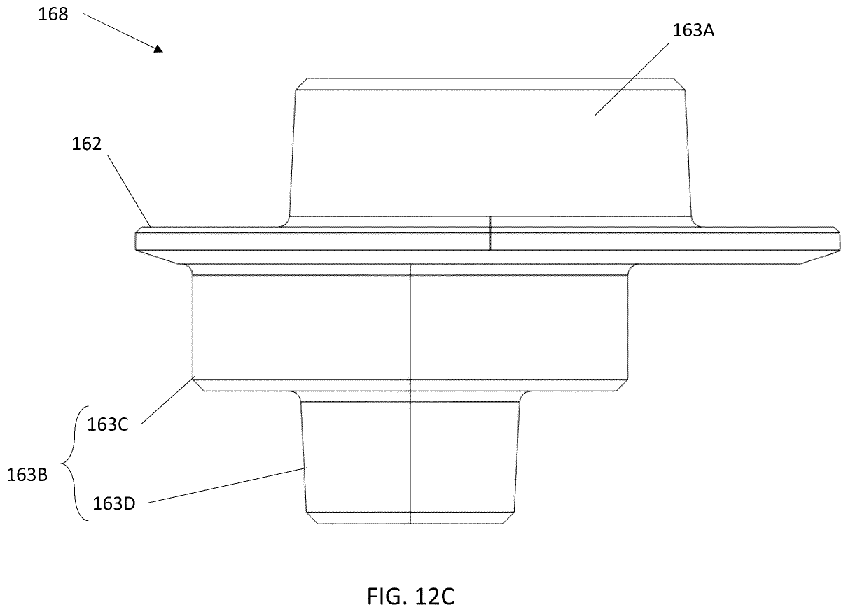

FIGS. 12A-12C illustrate an embodiment of an adapter or coupler 168. As described above, the coupler 168 can include the proximal extension 163A configured to connect to the articular body 164 and the distal extension 163B. In some configurations, the distal extension 163B can include a first distal portion 163C and a second distal portion 163D. The second distal portion 163D can extend distally from the first distal portion 163C. In some configurations, the first distal portion 163C may have a greater diameter than the second distal portion 163D. The first distal portion 163C can be configured to engage within the second recess 54, 254, 454, 554, 564 of the fracture stem 30, 230 or the spacer 150A, 150B, 150C. The second distal portion 163D can be configured to engage within the first recess 52, 252, 452, 552, 562 of the fracture stem 30, 230 or the spacer 150A, 150B, 150C. The disc or middle portion 162 can be disposed between the proximal extension 163A and the distal extension 163B. The disc or middle portion 162 can contact the peripheral rim 38, 238, 438, 538, 638 of the fracture stem 30, 230 or the spacer 150A, 150B, 150C. In some configurations, the disc or middle portion 162 can provide a spacer function in use when the adapter or coupler 168 is coupled to the stem 30, 230. In some configurations, the disc or middle portion 162 may include a window 165. The window 165 can uncover an indicium on a corresponding stem that is indicative of an orientation or a configuration of the articular body 164 relative to the other member of the joint prosthesis (e.g., the anchor 103, 113 or to a glenoid component) or a native glenoid in the case of a hemi-arthroplasty. The adapter or coupler 168 may also include a channel 165 extending between a proximal end 165A and a distal end 165B.

FIGS. 13A and 13B illustrates an embodiment of a screw 170 that can be received by the plurality of apertures 62, 64, 98, 262, 264, 265, 267, 269, 298 of the stem 230. The screw 170 may have a length greater than a width of the screw 170. The length of the screw 170 may be greater the length of the plurality of apertures 62, 64, 98, 262, 264, 265, 267, 269, 298. For example, when the screw 170 is inserted into an aperture of the plurality of apertures 62, 64, 98, 262, 264, 265, 267, 269, 298, a portion of screw 170 may be inserted into the bone of the patient. The screw 170 can be configured to secure the stem 30, 230 to the bone of the patient. In some configurations, the screw 170 can have a continuous thread 176 between a proximal head 172 of the screw 170 and a distal end 174 of the screw 170. In some configurations, the screw 170 can include one or more cutouts 178 at the distal end of the screw 170. The one or more cutouts 178 can extend from the distal end 174 of screw 170 to at least a portion of the thread 176. Advantageously, the screw 170 can be configured to screw into, for example, the bone of the patient faster due to the one or more cutouts 178 compared to a screw 170 without the one or more cutouts 178.

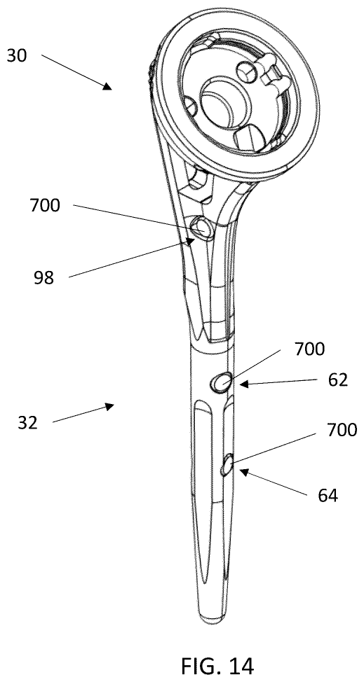

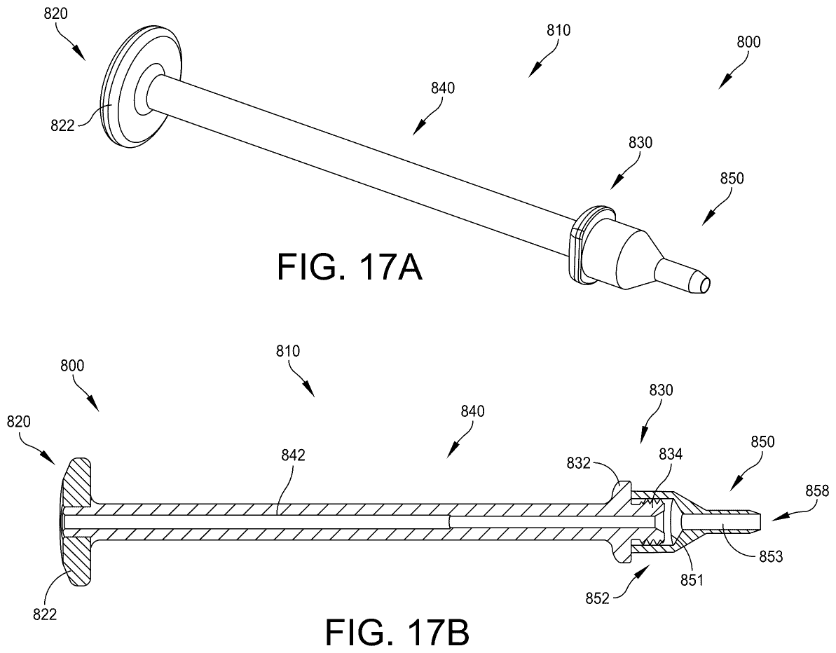

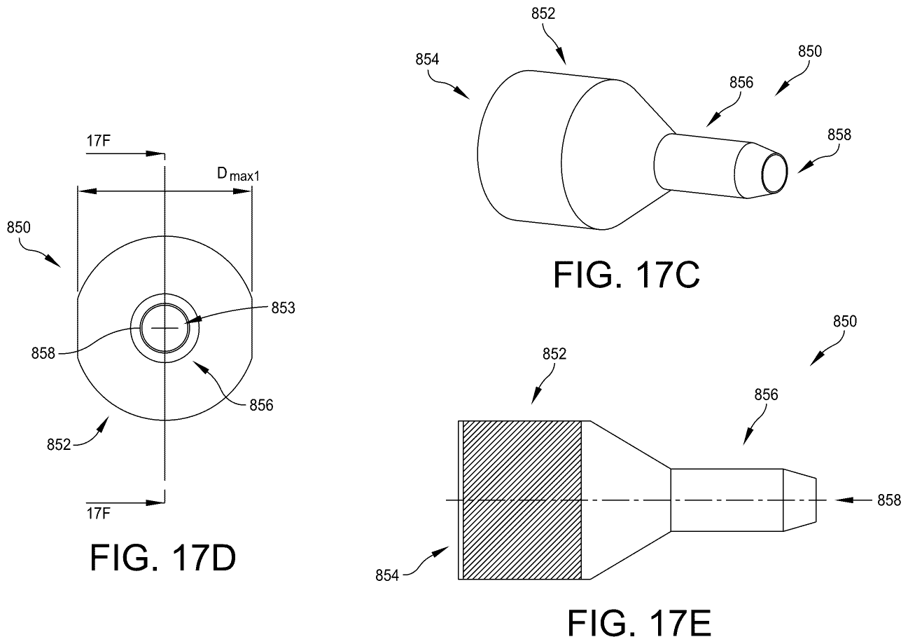

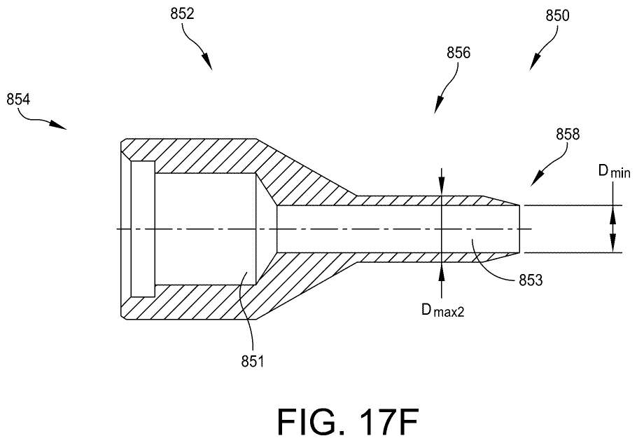

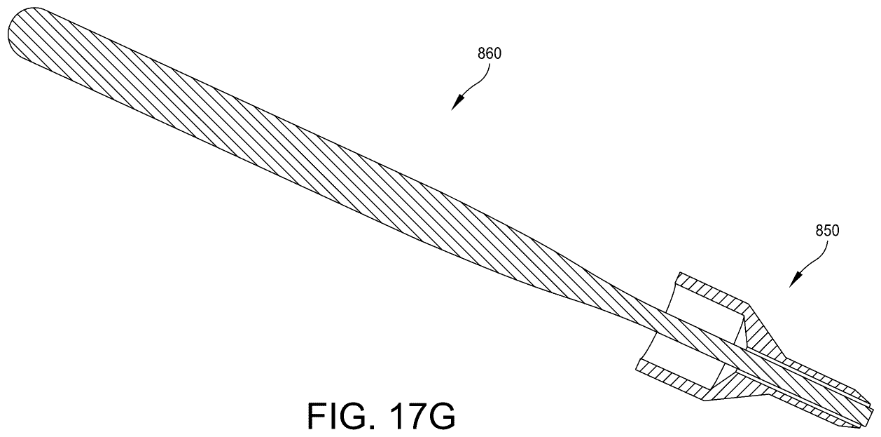

FIGS. 14-16 illustrate various embodiments of a plug 700A, 700B. FIG. 14 illustrates the stem 30 with one or more plugs 700 in each aperture 62, 64, 98. The one or more plugs 700 can include an elongate plug 700A and/or a plug 700B, shown in Figures and 16, respectively. When the stem 30 is being used in cemented applications, the one or more plugs 700 can prevent cement from bridging across an aperture of the plurality of apertures 62, 64, 98. The one or more plugs 700 can comprise a polyethylene material, a bone graft, or a combination thereof. For example, the clinician can use a graft tool 800, which is further described below in relation to FIGS. 17A-17C, to create one or more plugs 700.

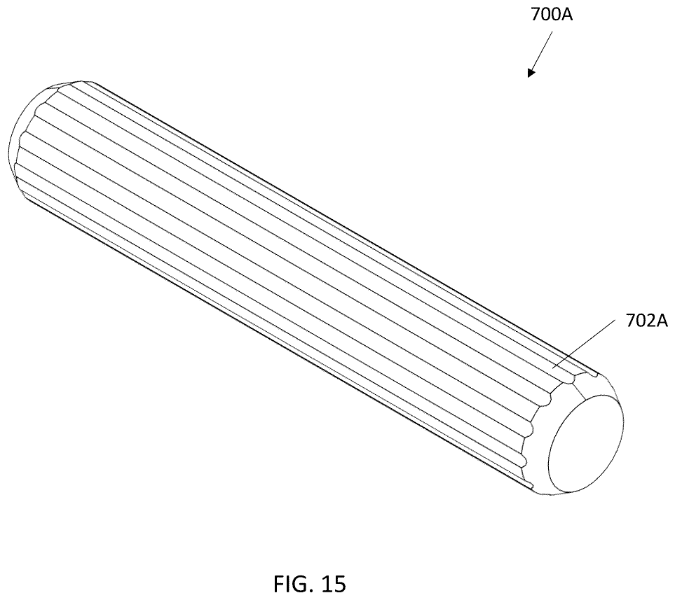

As shown in FIG. 15, the elongate plug 700A can include a length greater than a width of the elongate plug 700A. For example, the length and the width of the elongate plug 700A can be similar to or the same as the length and width of an aperture of the plurality of apertures 62, 64, 98 such that a single elongate plug 700A can be received by the aperture. In some configurations, the length of the elongate plug 700A can be between about 10 mm and about 40 mm, about 20 mm and about 30 mm, or about 25 mm. In some configurations, the width of the elongate plug 700A can be between about 2 and about 10 mm, or about 4.4 mm. In some configurations, the width of the elongate plug 700A can be between about 5% and about 30%, about 10% and about 20%, or about 18% of the length of the elongate plug 700A. The elongate plug 700A may also include a plurality of slots 702A that extend along the length of the elongate plug 700A and are circumferentially spaced apart. A length of each of the plurality of slots 702A may be less than or equal to the length of the elongate plug 700A. A width of each of the plurality of slots 702A can be between about 0.1 mm and about 1.0 mm, or about 0.5 mm.

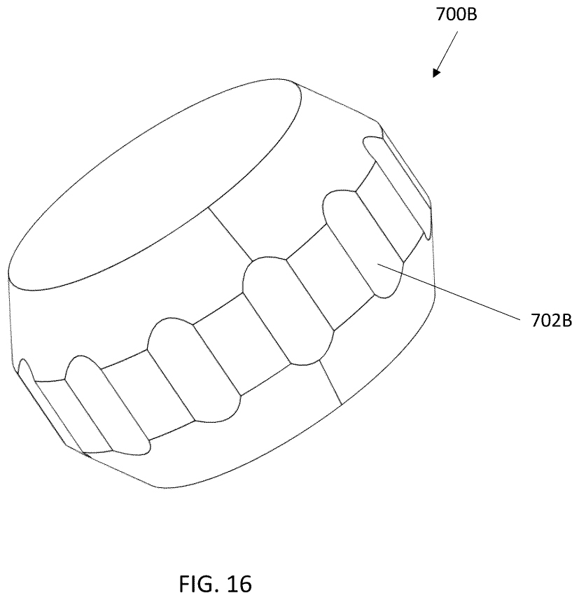

As shown in FIG. 16, the plug 700B can include a width greater than a length of the plug 700B. For example, the length of the plug 700B can be less than the length of an aperture of the plurality of apertures 62, 64, 98 and the width of the plug 700B can be similar to or the width of the aperture of the plurality of apertures 62, 64, 98 such that multiple plugs 700B (e.g., two, three, four or more) can be received by the aperture. In some configurations, the length of the plug 700B can be between about 1.0 mm and about 4.0 mm, about 2.0 mm and about 3.0 mm, or about 2.5 mm. In some configurations, the width of the plug 700B can be between about 2 and about 10 mm, or about 4.4 mm. In some configurations, the length of the plug 700B can be between about 30% and about 70%, about 40% and about 60%, or about 56% of the width of the plug 700B. The plug 700B may also include a plurality of slots 702B that extend along the length of the plug 700B and are circumferentially spaced apart. A length of each of the plurality of slots 702B may be less than or equal to the length of the plug 700B. A width of each of the plurality of slots 702B can be between about 0.1 mm and about 1.0 mm, or about 0.5 mm.

It may be desirable, for example, to use the elongate plug 700A for ease of handling and inserting into one or more of the plurality of apertures 62, 64, 98. On the other hand, it may be desirable to use one or more of the plugs 700B (e.g., two, three, four, five or more plugs 700B) to fill the aperture(s) 62, 64, 98 without needing to cut the length of the plug 700B. Methods of using the plugs 700A, 700B are further described below in relation to FIGS. 18E-18H, 19F, 20E, and 21E.

FIGS. 17A-17F illustrates a graft tool 800 that can be used to create the one or more plugs 700 out of bone. The graft tool 800 can include an impactor 810 and a tip 850. The impactor 810 can include a distal end 820, a proximal end 830, and a middle portion 840 extending between the two ends 820, 830. The distal end 820 can include an impaction head 822 having a larger diameter than the middle portion 840 and/or the proximal end 830. The impaction head 822 can be configured to receive impaction forces from a tool (e.g., a mallet). The proximal end 830 can include a first portion 832 and a second portion 834 proximal to the first portion 832. A perimeter of the first portion 832 can extend beyond a perimeter of the middle portion 840. A diameter or perimeter of the second portion 834 can be similar to or greater than the diameter or perimeter of the middle portion 840. The impactor 810 can also include a channel 842 that extend from the distal end 820 to the proximal end 830.

FIGS. 17C-17F illustrate different views of the tip 850. As shown in FIG. 17C, the tip 850 can include a distal portion 852 with a distal end 854 and a proximal portion 856 with a proximal end 858. The distal portion 852 can have a greater diameter than the proximal portion 856. In some configurations, the distal portion 852 may have a varying diameter. For example, a diameter of the distal end 854 of the distal portion 852 can be greater than a diameter of a proximal end of the distal portion 852. As shown in FIG. 17D, in some configurations, a maximum diameter Dₘₐₓ₁ of the distal portion 852 can be between about 5 mm and about 30 mm, about 10 mm and about 20 mm, or about 15.9 mm, or about 16.9 mm. In some configurations, the distal portion 852 can have a funnel-like shape. The distal portion 852 of the tip 850 can be configured to couple to the proximal end 830 of the impactor 810. For example, as shown in FIG. 17B, the distal portion 852 of the tip 850 can include a recess 851 that can be threaded and the second portion 834 of the proximal end 830 of the impactor 810 can have corresponding threads to engage with the threaded opening of the tip 850. The recess 851 can extend from the distal end 854 of the tip 850 toward the proximal end 858 of the tip 850. In some configurations, a length of the recess 851 can be less than or equal to the length of the distal portion 852. In some configurations, when the impactor 810 is coupled to the tip 850, a proximal facing surface of the first portion 832 of the proximal end 830 of the impactor 810 can abut a distal facing surface of the distal end 854 of the tip 850.

FIG. 17F illustrates a cross sectional view of the tip 850 along the line 17F-17F in FIG. 17D. FIG. 17F illustrates example dimensions of the tip 850. The proximal portion 856 of the tip 850 may have a diameter less than the diameter of the distal portion 852. In some configurations, the proximal portion 856 may have a varying diameter. For example, a diameter of a distal end of the proximal portion 856 can be greater than a diameter of the proximal end 858 of the proximal portion 856. In some configurations, a maximum diameter Dₘₐₓ₂ of the proximal portion can be between about 2 mm and about 20 mm, about 5 mm and about 10 mm, or about 6.4 mm. In some configurations, a minimum diameter Dₘᵢₙ of the proximal portion can be between about 2 mm and about 20 mm, about 5 mm and about 10 mm, or about 4.4 mm. The diameter of the distal end of the proximal portion 856 can be the same as or similar to the diameter of the proximal end of the distal portion 852. The proximal portion 856 can have a channel 853 that extends from the proximal end 858 toward the distal end 854. A length of the channel 853 can be greater than or equal to the length of the proximal portion 850. In some configurations, the channel 853 can at least partially extend into the distal portion 852.

In use, the clinician can thread the impactor 810 into the tip 850. The clinician can position the proximal end 858 of the tip 850 against the bone of a patient. The clinician can use a mallet to apply impacting forces to the impaction head 822, which causes portions of the bone to fill the channel 853 of the tip 850. Once a sufficient amount of bone is in the channel 853, the clinician can remove the bone graft from the tip 850. As shown in FIG. 17G, the clinician can remove the tip 850 from the impactor 810 and use a tool 860, such as a screwdriver or a rod with a flat end, to remove the bone graft from the tip 850 by pushing the tool 860 against one end of the bone graft until the bone graft exits the proximal end 858 of the tip 850. The clinician can remove the bone graft from the tip 850 by inserting a tool, such as a pin or a rod with a flat end, into the channel 842 of the impactor 810 and push the bone graft until the bone graft exits the proximal end 858 of the tip 850. Advantageously, using the rod with the flat end can reduce or prevent breaking in the bone graft. Once the bone graft is removed, the clinician can insert the bone graft into one or more of the apertures 62, 64, 98, 262, 264, 265, 267, 269 of the stem 30, 230.

### IV. Shoulder Arthroplasty Methods and Instrumentation

The humeral anchors described above can be implanted using certain tools and instruments that are described below in connection with FIGS. 18A-21E.

### A. Dual Use Surgical Instruments

One advantage of various kits and systems disclosed herein is that multiple different types of humeral anchors can be implanted using shared instrumentation. Examples of shared instrumentation are discussed below.

1. Stem Holder

As discussed above, a stem 30, 230 may include one or more interfacing features, such as the one or more apertures 51a, 51b, 53, 251a, 251b, 253, configured to engage a tool and enable insertion of the stem 30, 230 into the bone. FIGS. 18A-18I illustrate a stem holder 900 configured to position a stem 30, 230 into the bone (e.g., the humerus H). As discussed in more detail below, the stem holder 900 can be configured to receive impaction forces, for example from a mallet, to properly insert the stem 30, 230 into the bone. For example, the proximal surface of the stem 30, 230 (e.g., the stem face 250) can take most of the impaction force via direct contact with a distal surface 903 of the stem holder 900.

The stem holder 900 may include an elongate body 905. The elongate body 905 may generally extend from a first or proximal end 902 of the stem holder 900 to a second or distal end 904 of the stem holder 900. As shown in FIG. 18B, the elongate body 905 may include a stem interfacing portion 910 at the second end 904 of the stem holder 900. The stem interfacing portion 910 may be configured engage the stem face 50, 250 of a stem 30, 230. For example, the stem interfacing portion 910 can include one or more interfacing features 911, 912, 913, 914. The plurality of interfacing features can include a first interfacing feature 911, a second interfacing feature 913, a third interfacing feature 912, and/or a fourth interfacing feature 914, which is further described below in relation to FIG. 18C. The third interfacing feature 912 can extend distally from a distal surface 903 of the stem holder 900 and be configured to engage with the second recess 54, 254 of the stem 30, 230. For example, the second recess 54, 254 can receive the third interfacing feature 912. In some configurations, the third interfacing feature 912 can be spaced from the perimeter of the distal surface 903 such that a portion of the distal surface 903 can contact the peripheral rim 38, 238 of the stem face 50, 250 when the stem 30, 230 is coupled to the stem holder 900. In some configurations, the third interfacing feature 912 can include two portions that are connected on one end and otherwise spaced apart. The gap between the two portions of the third interfacing feature 912 can have a width corresponding with a width of the fourth interfacing feature 914 such that the fourth interfacing feature 914 can be received by the gap between the two portions. The first and second interfacing features 911, 913 can extend distally from a distal surface of the third interfacing feature 912. For example, the first interfacing feature 911 can extend from the distal surface of one of the two portions of the third interfacing feature 912 and the second interfacing feature 913 can extend from the distal surface of the other one of the two portions of the third interfacing feature 912. The first and second interfacing features 911, 913 can be configured to engage with the one or more apertures 51a, 51b, 53, 251a, 251b, 253 of the stem 30, 230. For example, the first interfacing feature 911 can be received by the second aperture 51b of the stem face 50, 250 and the second interfacing feature 913 can be received by the first aperture 51a of the stem face 50, 250.

The stem holder 900 may also include a moveable assembly 906 (see FIGS. 18C and 18D) coupled with the elongate body 905. FIG. 18D illustrates the stem holder 900 with the elongate body 905 partially transparent such that the internal components (e.g., the moveable assembly 906) are visible. The moveable assembly 906 may include a handle 908 disposed between the first end 902 and the second end 904 of the stem holder 900. The handle 908 may be coupled, for example pivotably coupled, with the elongate body 905 at pivot location 918.

As shown in FIGS. 18C and 18D, the moveable assembly 906 may also include the fourth interfacing feature 914 disposed at the second end 904 of the stem holder 900. The fourth interfacing feature 914 may be coupled, for example pivotably coupled, with the elongate body 905 at pivot location 919. In some configurations, the fourth interfacing feature 914 may be a stationary peg that is fixed with respect to the remainder of the stem holder 900 and does not move. The fourth interfacing feature 914 may be configured to engage with one of the one or more apertures 51a, 51b, 53, 251a, 251b, 253 of the stem 30, 230. For example, the fourth interfacing feature 914 may be a peg configured to interface with the anti-rotation aperture 53, 253.

The handle 908 may be directly or indirectly coupled to the fourth interfacing feature 914. For example, the handle 908 may be indirectly coupled to the fourth interfacing feature 914 by a spring linkage 916. The spring linkage 916 may have an arcuate portion and a spring gap 920. The spring linkage 916 may be indirectly coupled to the elongate body 905 by the handle 908 and/or the fourth interfacing feature 914 without a direct connection between the spring linkage 916 and the elongate body 905.

The handle 908 can be configured to move the fourth interfacing feature 914 between a first configuration and a second configuration. A proximal end of the handle 908 can be free to move relative to the elongate body 905. The transition between the first configuration and the second configuration may include rotation and/or translation of the fourth interfacing feature 914 with respect to elongate body 905. For example, actuating (e.g., pivoting) the handle 908 away from the elongate body 905 may move the fourth interfacing feature 914 from the first configuration to the second configuration, while releasing the handle 908 may move the fourth interfacing feature 914 back to the first configuration. In the second configuration, the fourth interfacing feature 914 can be rotated and at least partially retracted with respect to a distal surface 903 of the stem holder 900. In this position, the surgeon may engage the stem face 50, 250 of the stem 30, 230. While the fourth interfacing feature 914 engages the stem face 50, 250 of the stem 30, 230, the handle 908 may be released (e.g., toward the elongate body 905) so as to apply a gripping force to the stem 30, 230. In the first configuration, the spring linkage 916 can be compressed (e.g. the spring gap 920 has been slightly closed), and provide a spring force which can help to hold the fourth interfacing feature 914 closed against the stem 30, 230.

In some configurations, the fourth interfacing feature 914 may be angled relative to the first and second interfacing features 911, 913. For example, longitudinal axes of the first and second interfacing features 911, 913 may extend substantially perpendicularly from the distal surfaces of the third interfacing feature 912. Accordingly, a longitudinal axis of the fourth interfacing feature 914 may be angled relative to the longitudinal axes of the first and second interfacing features 911, 913. When the fourth interfacing feature 914 is moved from the first configuration to the second configuration, the angle between the fourth interfacing feature 914 and the first and second interfacing features 911, 913 can decrease. In some configuration, the angle between the fourth interfacing feature 914 and the first and second features 911, 913 may increase when moving the fourth interfacing feature 914 from the first configuration to the second configuration.

Stem holder 900 may include at least one impaction head 924 configured to receive impaction forces from a tool (e.g., a mallet). For example, the stem holder 900 may include a single impaction head 924 that may be disposed at the first end 902 of the stem holder 900. In some configurations, the at least one impaction head can include two impaction heads with the impaction head 924 and a second impaction head being positioned closer to the second end 904 of the stem holder 900. The impaction head 924 may be coupled with the elongate body 905. In some configurations, the impaction head 924 may be aligned with the longitudinal axis of the elongate body 905. In some configurations, the impaction head 924 may be disposed at an angle relative to the longitudinal axis of the elongate body 905. When a force is applied to the impaction head 924, the impacting force can be directed to the stem 30, 230 in a direction aligned with a longitudinal axis of the stem 30, 230 to embed the stem 30, 230 in the bone.

The stem holder 900 may also be configured to receive a retroversion rod. For example, the retroversion rod may be inserted into one of the openings 926. Each opening may position the retroversion rod at a different angle, corresponding to the desired angle of resection, and allow the surgeon to evaluate the version. If the proximal bone resection was not accurate or for other reasons dictated by surgeon judgment, the surgeon can modify the resection plane.

The stem holder 900 may also include a height gauge 930 configured to determine a height of the stem 30, 230 relative to the humerus or a depth of the stem 30, 230 within the humerus. For example, prior to implanting the stem 30, 230 into the humerus, a clinician can determine the appropriate stem height of the stem 30, 230 relative to the humerus based on x-rays of the humerus, a trial stem, or other suitable methods. The height gauge 930 can include a ruler 932, a connector rod 934, a connector hub 940, and a marker 950. The ruler 932 can include a plurality of markings (not shown) associated with a measurement (e.g., millimeters (mm), centimeters (cm)). In some configurations, the ruler 932 can have an elongate shape (e.g., a cylinder). In some configurations, a longitudinal axis of the ruler 932 can be substantially parallel to the longitudinal axis of the elongate body 905. The connector rod 934 can be configured to couple the height gauge 930 to the elongate body 905. In some configurations, the elongate body 905 can include a connector portion 928 configured to receive the connector rod 934. The elongate body 905 can include the connector portion 928 on one or both sides of the elongate body 905. When the elongate body 905 has connector portion 928 on both sides, the clinician can position the connector rod 934 in either of the connector portions 928. The connector rod 934 can have an elongate shape (e.g., a cylindrical shape). In some configurations, a longitudinal axis of the connector rod 934 can be substantially perpendicular to the longitudinal axis of the ruler 932 and/or the elongate body 905.

The marker 950 can extend perpendicularly from a distal end of the ruler 932. A distal facing surface of the marker 950 can be configured to be positioned on the humerus after the humeral head is removed. The connector hub 940 may be configured to couple the ruler 932 and the connector rod 934. The connector hub 940 can include an adjustment portion 942 and a connector portion 944. The adjustment portion 942 can be configured to move the ruler 932 relative to the connector rod 934. In some configurations, the adjustment portion 942 can be a wheel 942. In use, after the clinician has determined the appropriate stem height and connected the stem 30, 230 to the stem holder 900, the clinician can turn the wheel 942 to move the ruler 932 until the ruler 932 reaches the appropriate stem height. The clinician can apply impaction forces to the impaction head 924 to insert the stem 30, 230 into the humerus until the marker 950 contacts the resected portion of the humerus.

The stem holder 900 may form part of a kit including a stemless bone anchor and/or a stemmed bone anchor. The stemless and/or stemmed bone anchor may include any of the features of the implants described above. The stem interfacing portion 910 may be configured to engage the stem holder interface of the stemless bone anchor and/or the stem face of the stemmed bone anchor.

In use, the same stem holder 900 may engage the stem holder interface of a first, stemless bone anchor or the stem face of a second, stemmed bone anchor. The stemless and/or stemmed bone anchor may include any of the features of the implants described above. For example, the stem holder 900 may engage the stem holder interface of the stemless bone anchor and advance the stemless bone anchor into bone matter exposed at a resection of a bone. When advancing the stemless bone anchor, a force may be applied to the impaction head 924 of the stem holder 900 to apply a force perpendicular to the resection plane of the bone.

The same stem holder 900 may engage the stem face of the stemmed bone anchor and advance the stemmed bone anchor to position the stem of the bone anchor in a medullary canal of the bone. When advancing the stemmed bone anchor, a force may be applied to the impaction head 924 of the stem holder 900 to apply a force aligned with a longitudinal axis of the stemmed bone anchor to embed the stem in the bone.

2. Jig

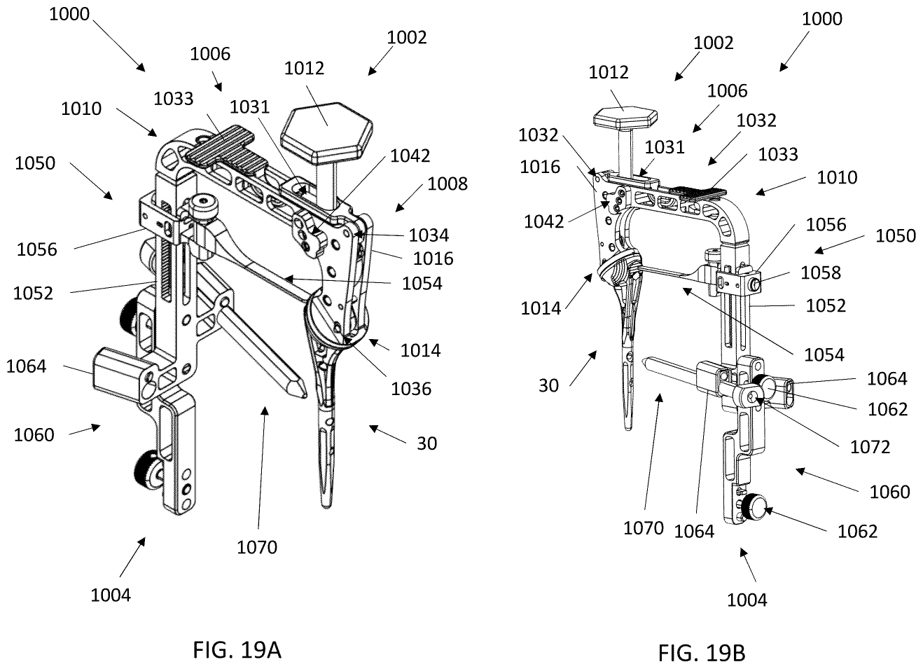

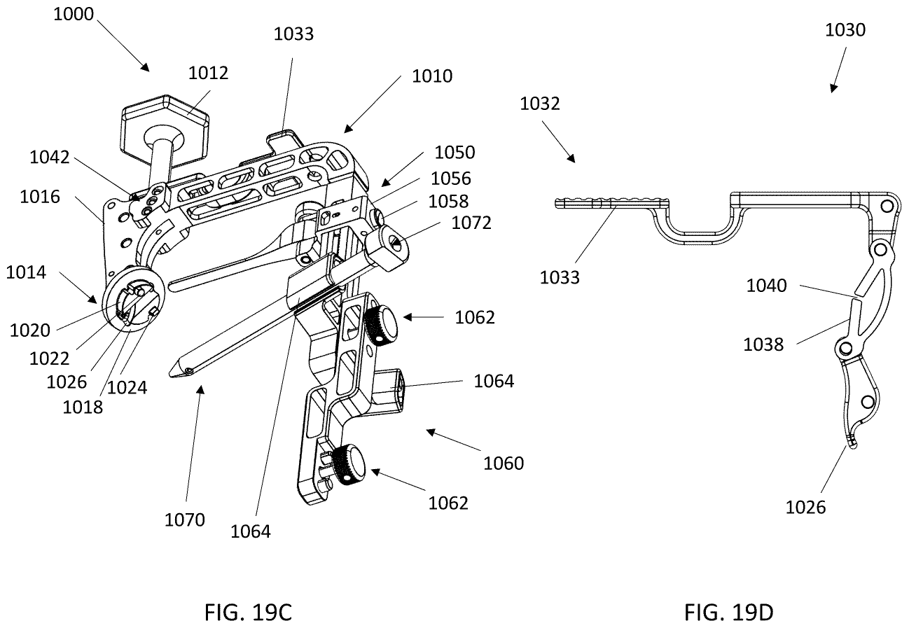

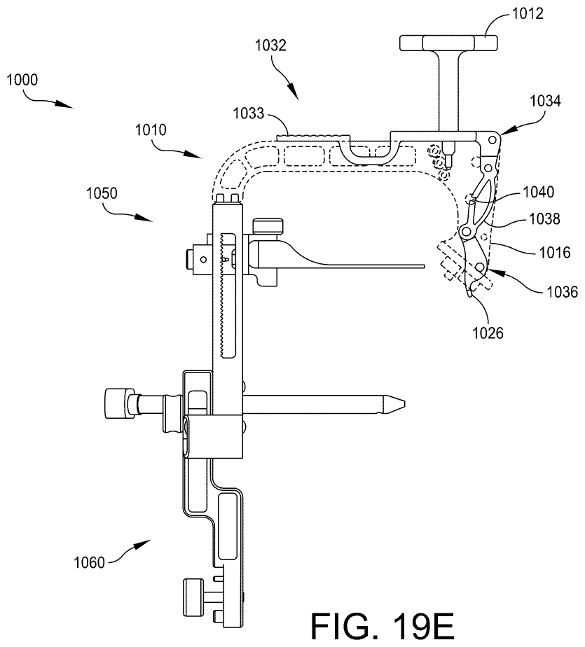

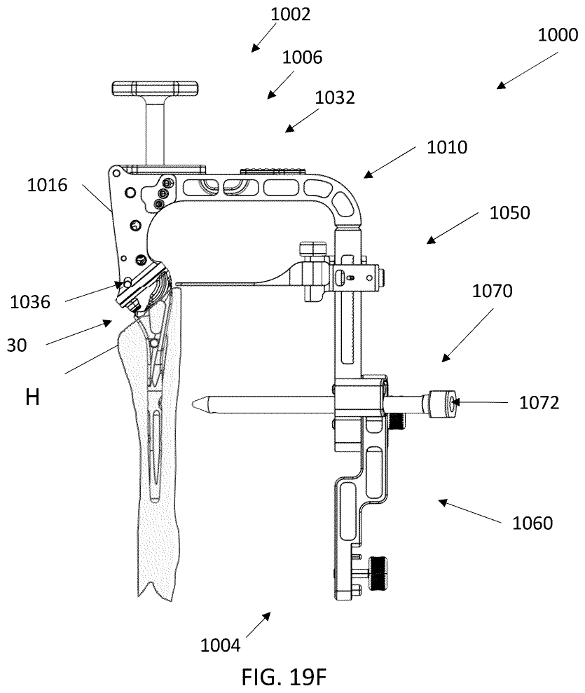

FIGS. 19A-21E illustrate different configurations of a jig 1000, 1100, 1200 configured to position a stem 30, 230 into the bone (e.g., the humerus H). As discussed in more detail below, the jig 1000, 1100, 1200 can be configured to receive impaction forces, for example from a mallet, to properly insert the stem 30, 230 into the bone. For example, the proximal surface of the stem 30, 230 (e.g., the stem face 50, 250) can take most of the impaction force via direct contact with a distal surface 1003, 1103, 1203 of the jig 1000, 1100, 1200.

FIGS. 19A-19F illustrates an embodiment of the jig 1000. The jig 1000 may extend between a first or proximal end 1002 and a second or distal end 1004. At or near the proximal end 1002, the jig can include a proximal portion 1006. The proximal portion 1006 can include an inserter portion 1008 and a connecting bridge 1010. The inserter portion 1008 can include at least one impaction head 1012 and an elongate body 1016.

The elongate body 1016 may generally extend from the impaction head 1012 toward the second end 1004 of the jig 1000. A longitudinal axis of the elongate body 1016 can be substantially perpendicular to a longitudinal axis of the connecting bridge 1010. The elongate body 1016 may include an interfacing portion 1014 at a distal end of the elongate body 1016. The interfacing portion 1014 may be configured engage the stem face 50, 250 of a stem 30, 230. The interfacing portion 1014 can be the same as or similar to the stem interfacing feature 910 described in relation to FIG. 18B. For example, as shown in FIG. 19C, the interfacing portion 1014 can include a plurality of interfacing features 1020, 1022, 1024, 1026 extending from a distal facing surface 1018 of the interfacing portion 1014. The plurality of interfacing features can include a first interfacing feature 1022, a second interfacing feature 1024, a third interfacing feature 1020, and a fourth interfacing feature 1026.

The jig 1000 may also include a moveable assembly 1030 (see FIGS. 19D and 19E) coupled with the elongate body 1016. FIG. 19E illustrates the jig 1000 with the elongate body 1016 and the connecting bridge 1010 partially transparent such that the internal components (e.g., the moveable assembly 1030) are visible. The moveable assembly 1030 may include a handle 1032 disposed along the connecting bridge 1010. The handle 1032 may be coupled, for example pivotably coupled, with the elongate body 1016 at pivot location 1034.

As shown in FIGS. 19D and 19E, the moveable assembly 1030 may also include the fourth interfacing feature 1026 disposed at the distal end of the elongate body 1026. The fourth interfacing feature 1026 may be coupled, for example pivotably coupled, with the elongate body 1026 at pivot location 1036. In some configurations, the fourth interfacing feature 1026 may be a stationary peg that is fixed with respect to the remainder of the stem holder 1000 and does not move. The fourth interfacing feature 1026 may be configured to engage with one of the one or more apertures 51a, 51b, 53, 251a, 251b, 253 of the stem 30, 230. For example, the fourth interfacing feature 1026 may be a peg configured to interface with the anti-rotation aperture 53, 253.

The handle 1032 may be directly or indirectly coupled to the fourth interfacing feature 1026. For example, the handle 1032 may be indirectly coupled to the fourth interfacing feature 1026 by a spring linkage 1038. The spring linkage 1038 may have an arcuate portion and a spring gap 1040. The spring linkage 1038 may be indirectly coupled to the elongate body 1016 by the handle 1032 and/or the fourth interfacing feature 1026 without a direct connection between the spring linkage 1038 and the elongate body 1016.

The handle 1032 can be configured to move the fourth interfacing feature 1026 between a first configuration and a second configuration. A free end 1033 of the handle 1032 can be free to move relative to the elongate body 1016. The transition between the first configuration and the second configuration may include rotation and/or translation of the fourth interfacing feature 1026 with respect to elongate body 1016. For example, actuating (e.g., pivoting) the free end 1033 of the handle 1032 away from connecting bridge 1010 may move the fourth interfacing feature 1026 from the first configuration to the second configuration, while releasing the free end 1033 of the handle 1032 may move the fourth interfacing feature 1026 back to the first configuration. In the second configuration, the fourth interfacing feature 1026 can be rotated and at least partially retracted with respect to the distal surface 1018 of the interfacing portion 1014. In this position, the surgeon may engage the stem face 50, 250 of the stem 30, 230. While the fourth interfacing feature 1026 engages the stem face 50, 250 of the stem 30, 230, the free end 1033 of the handle 1032 may be released (e.g., toward the connecting bridge 1010) so as to apply a gripping force to the stem 30, 230. In the first configuration, the spring linkage 1038 can be compressed (e.g. the spring gap 1040 has been slightly closed), and provide a spring force which can help to hold the fourth interfacing feature 1026 closed against the stem 30, 230.

As show in FIGS. 19A and 19B, the handle 1032 may also include an elongate gap 1031. The elongate gap 1031 may be configured to receive a distal portion of the impaction head 1012 so that the distal portion of the impaction head 1012 can couple to the elongate body 1016 and/or the connecting bridge 1010.

The at least one impaction head 1012 can be configured to receive impaction forces from a tool (e.g., a mallet). For example, the jig 1000 may include a single impaction head 1012 that may be disposed at the first end 1002 of the jig 1000. In some configurations, the at least one impaction head can include two impaction heads with the impaction head 1012 and a second impaction head being positioned closer to the second end 1004 of the jig 1000. The impaction head 1012 may be coupled with the elongate body 1016. In some configurations, the impaction head 1012 may be parallel to or aligned with a longitudinal axis of the elongate body 1016. In some configurations, the impaction head 1012 may be disposed at an angle relative to the longitudinal axis of the elongate body 1016. When a force is applied to the impaction head 1012, the impacting force can be directed to the stem 30, 230 in a direction aligned with a longitudinal axis of the stem 30, 230 to embed the stem 30, 230 in the bone.

The jig 1000 may also be configured to receive a retroversion rod. For example, the retroversion rod may be inserted into one of the openings 1042. Each opening may position the retroversion rod at a different angle, corresponding to the desired angle of resection, and allow the surgeon to evaluate the version. If the proximal bone resection was not accurate or for other reasons dictated by surgeon judgment, the surgeon can modify the resection plane.

As shown in FIGS. 19A-19C, the jig 1000 may also include a height gauge 1050 configured to determine a height of the stem 30, 230 relative to the humerus or a depth of the stem 30, 230 within the humerus. The height gauge 1050 can be similar to or the same as the height gauge 930 of the stem holder 900. For example, the height gauge 1050 can include a ruler 1052 and a marker 1054. The ruler 1052 can include a plurality of markings (not shown) associated with a measurement (e.g., millimeters (mm), centimeters (cm)). In some configurations, the ruler 1052 can have an elongate shape. The ruler 1052 can have a substantially square cross-sectional shape. In some configurations, a longitudinal axis of the ruler 1052 can be substantially parallel to the longitudinal axis of the elongate body 1016. In some configurations, the longitudinal axis of the ruler 1052 can be substantially perpendicular to the longitudinal axis of the connecting bridge 1010. A proximal end of the ruler 1052 can be directly or indirectly coupled to the connecting bridge 1010.

The marker 1054 can extend perpendicularly from the ruler 1052. A distal facing surface of the marker 1054 can be configured to be positioned on the humerus after the humeral head is removed. In some configurations, the height gauge 1050 can include a marker connector 1056. The marker connector 1056 can include an aperture configured to receive the ruler 1052. The marker connector 1056 can be configured to couple the marker 1054 to the ruler 1052. The marker connector 1056 can include an adjustment portion 1058 configured to allow the marker 1054 and marker connector 1056 relative to the ruler 1052. In some configurations, the adjustment portion can be a release button 1058. In use, after the clinician has determined the appropriate stem height and connected the stem 30, 230 to the jig 1000, the clinician can turn the push the release button 1058 and move the marker connector 1056 until the marker connector 1056 and the marker 1054 reaches the appropriate stem height. The clinician can release the release button 1058 to secure a positon of the marker connector 1058 and marker 1054 relative to the ruler 1052. The clinician can apply impaction forces to the impaction head 1012 to insert the stem 30, 230 into the humerus until the marker 1054 contacts the resected portion of the humerus.

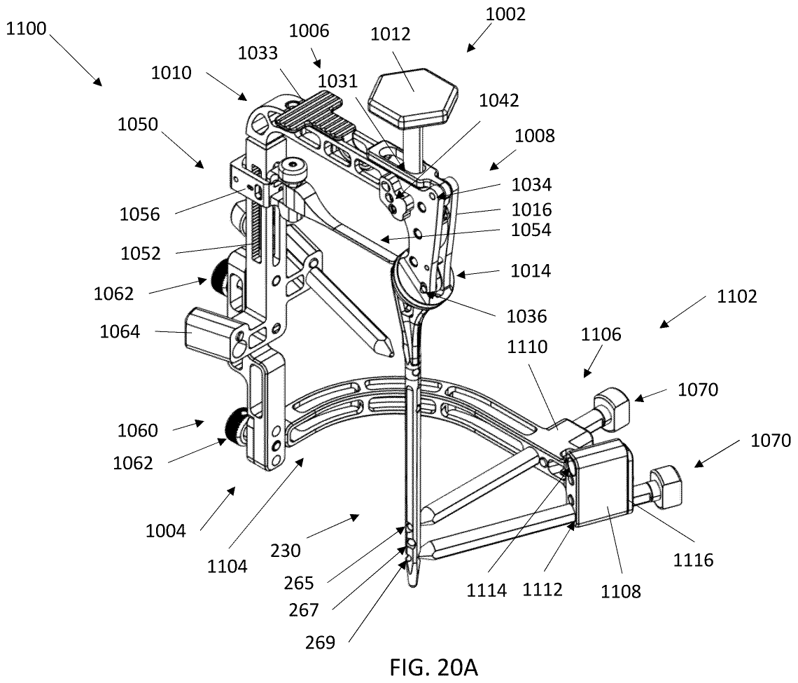

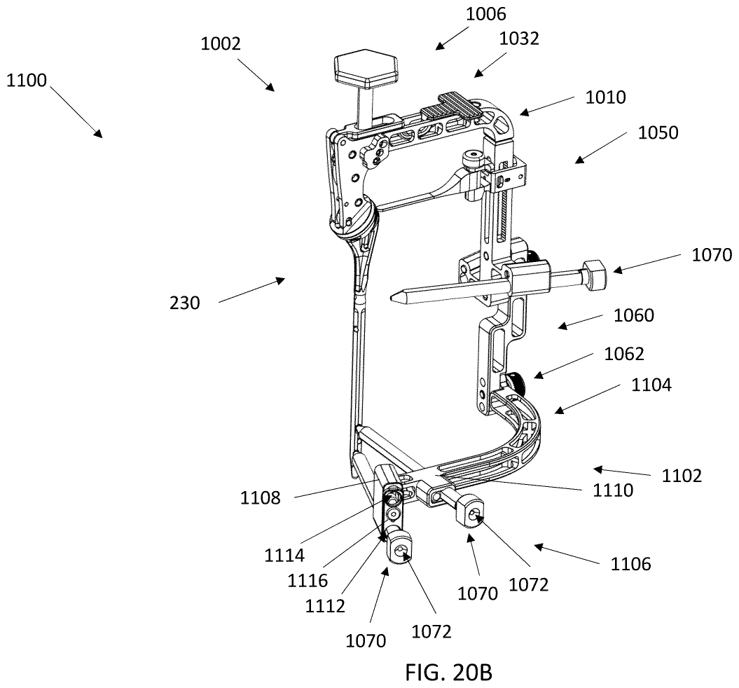

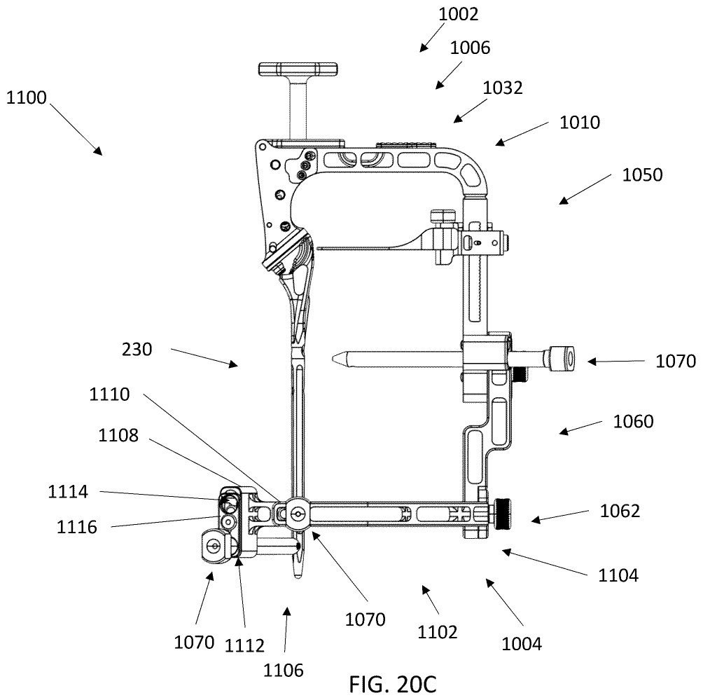

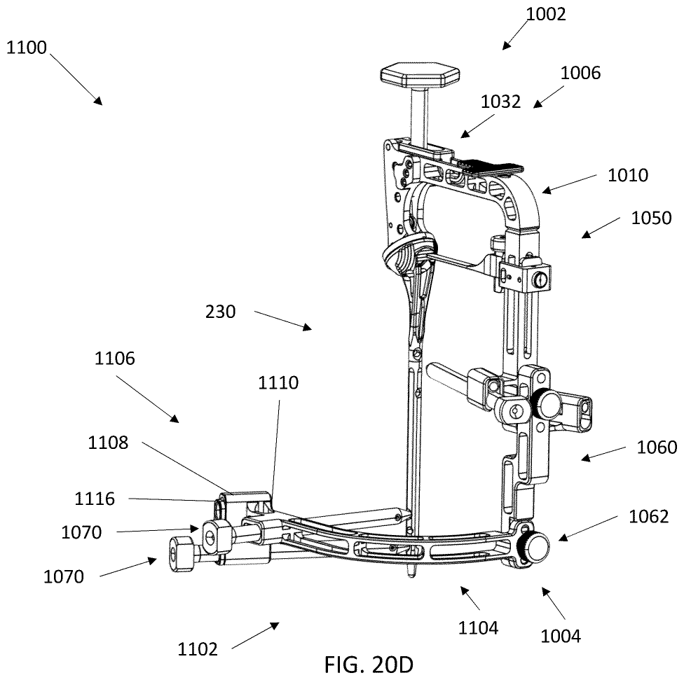

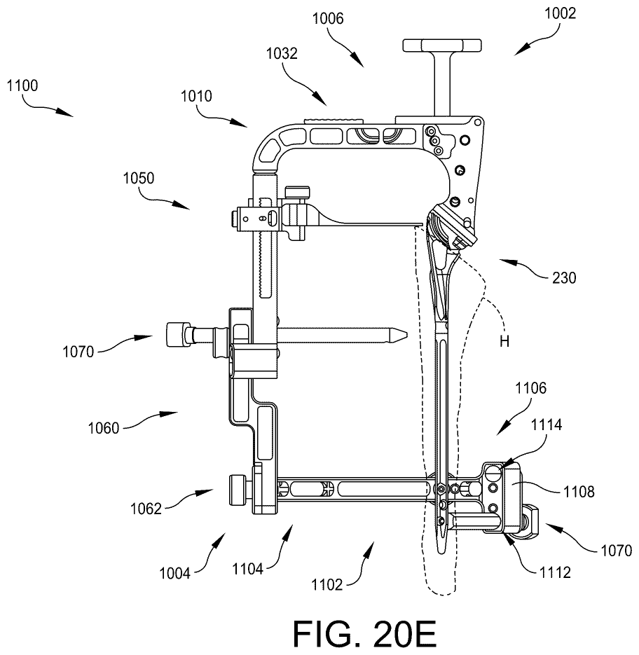

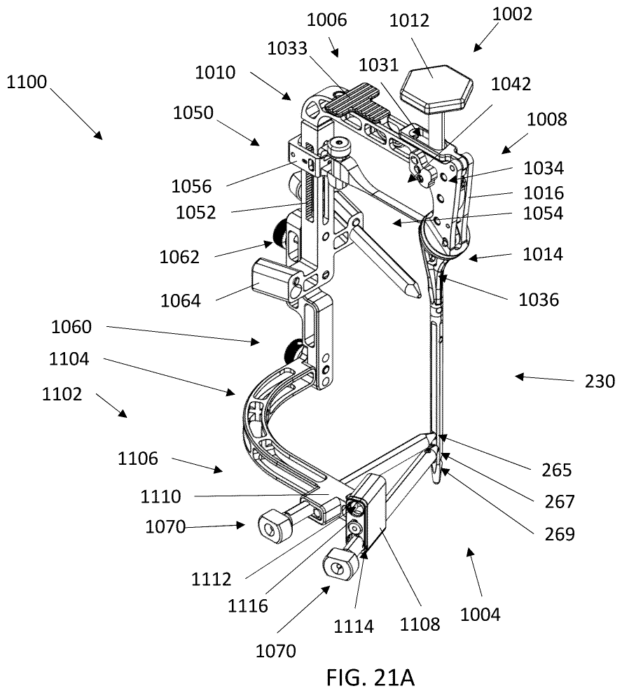

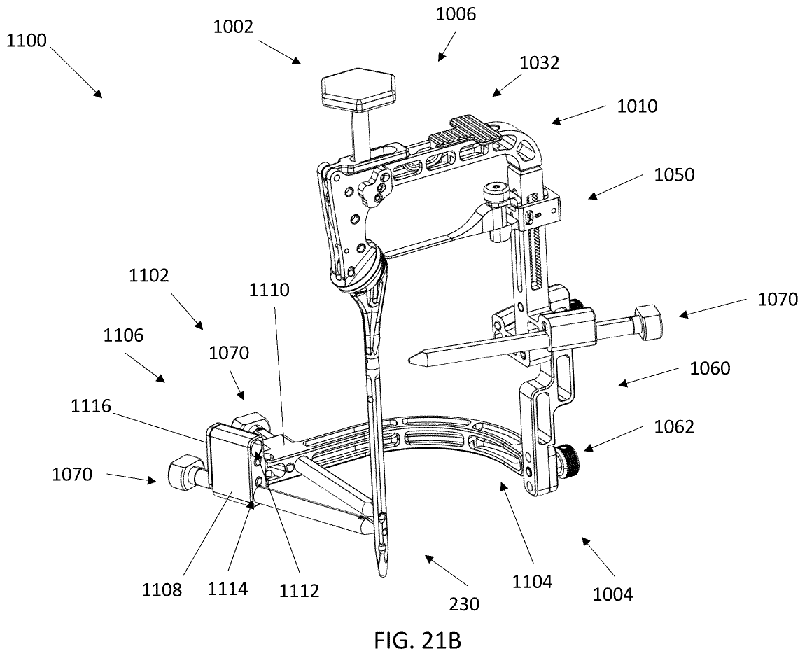

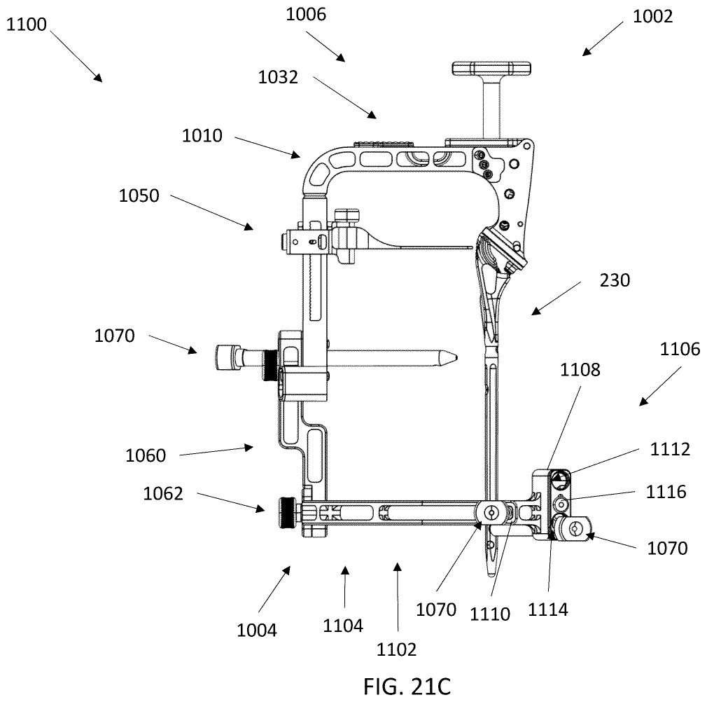

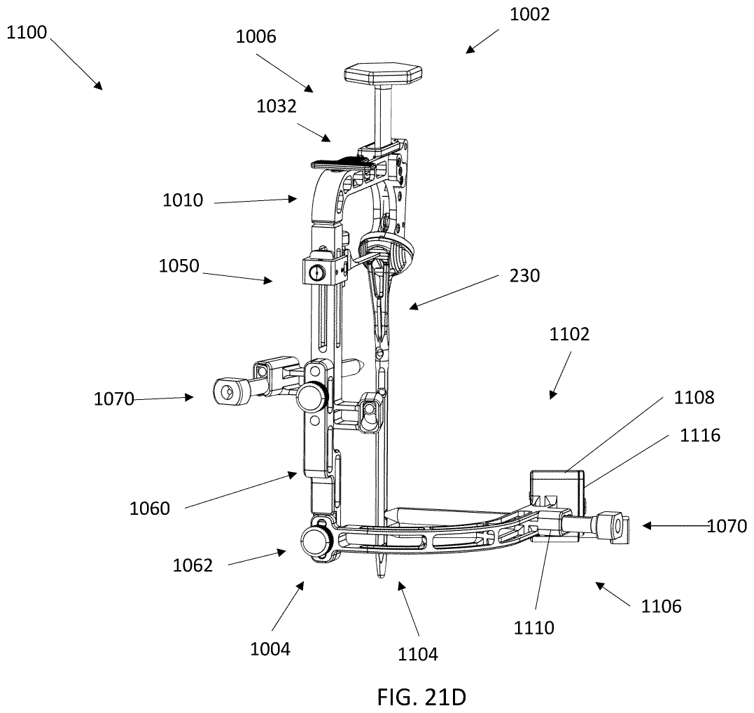

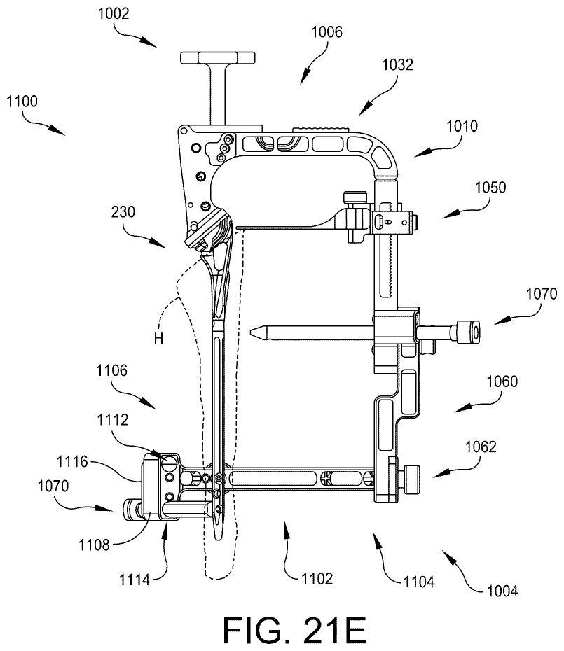

The jig 1000 may further include a vertical support structure 1060 and one or more screw guides 1064. The vertical support structure 1060 can extend from the height gauge 1050 to the distal end 1004 of the jig 1000. The vertical support structure 1060 can couple to the height gauge 1050 at a proximal end of the vertical support structure 1060. For example, the vertical support structure 1060 can be coupled to the height gauge 1050 by one or more fastening screws 1062. The vertical support structure 1060 can be configured to couple the jig 1000 with a distal arm extension 1102, which is further described below FIGS. 20A-21E.

The one or more screw guides 1064 can be configured to align one or more screws 170 with the one or more apertures 62, 64 in the distal shaft portion 32 of the stem 30. For example, as shown in FIGS. 19A-19C, each of the one or more screw guides 1064 can be configured to receive a drill sleeve 1070. The drill sleeve 1070 can include a channel 1072 that extends from a distal end (i.e., the end of the drill sleeve 1070 facing away from the screw 30) to a proximal end (i.e., the end of the drill sleeve 1070 facing the stem 30) of the drill sleeve 1070. The channel 1072 can be sized and shaped to receive a screw 170. For example, a surgeon can insert the screw 170 through the channel 1072 and drill the screw 170 through the one or more aperture 62, 64 in the distal shaft portion 32 of the stem to secure the stem 30 to the humerus of the patient. In some configurations, the drill sleeve 1070 can include a plurality of drill sleeves. The plurality of drill sleeves can be configured to nest inside one another.

FIGS. 20A-21E illustrate a first configuration (FIGS. 20A-20E) and a second configuration (FIGS. 21A-21E) of another embodiment of a jig 1100. The jig 1100 can include the jig 1000 described above in addition to a distal arm extension 1102. The distal arm extension 1102 can be configured to align one or more screws 170 with the one or more apertures 265, 267, 269 of the distal shaft 232 of the stem 230. The distal arm extension 1102 can include a first portion 1104 that includes a first end of the distal arm extension 1102 and a second portion 1106 that includes a second end of the distal arm extension 1102. The distal arm extension 1102 can include a curvature between the first and second ends. The first portion 1104 of the distal arm extension 1102 can be coupled to the distal end of the vertical support structure 1060. For example, the first end of the first portion 1104 can be secured to the distal end of the vertical support structure 1060 by a fastening screw 1062. In some configurations, the distal arm extension 1102 extend radially outward from the first end of the first portion 1104 to the second end of the second portion 1106.

The distal arm extension 1102 can be configured to be moveable between a first side of the jig 1100 (FIGS. 20A-20E) and a second side of the jig 1100 (FIGS. 21A-21E). For example, the clinician can loosen the fastening screw 1062 connecting the distal arm extension 1102 with the vertical support structure 1060 and rotate the distal arm extension 1102 about the distal end of the vertical support structure 1060. When the distal arm extension 1102 is on the appropriate side of the jig 1100, the clinician can tighten the fastening screw 1062 to secure the distal arm extension 1102 on the appropriate side of the jig 1100. The jig 1100 can be configured to implant a stem 230 into a left arm of a patient when the jig 1100 is on the first side of the jig 1110. The jig 1100 can be configured to implant the stem 230 into a right arm of the patient when the jig 1100 is on the second side of the jig 1110.

The distal arm extension 1102 can include one or more screw guides 1108, 1110. For example, the one or more guides 1108, 1110 can include a first screw guide 1108 and a second screw guide 1112. The first and second screw guides 1108, 1110 can be positioned on the second portion 1106 of the distal arm extension 1102. The first screw guide 1108 can be positioned at the second end of the second portion 1106. The second screw guide 1110 can be positioned between the second end of the second portion 1104 of the distal arm extension 1102 and the first portion 1104 of the distal arm extension 1102. For example, the second screw guide 1110 can be adjacent the first screw guide 1108. The first and second screw guides 1108, 1110 can be configured to align a screw 170 with one or more of the apertures 265, 267, 269 of the stem 240. For example, the first screw guide 1108 can align a screw 170 with the fourth or fifth aperture 267, 259 and the second screw guide 1110 can be configured to align the screw 170 with the third aperture 265. In some configurations, as shown in FIGS. 20A-21E, each of the first and second screw guides 1108, 1110 can be configured to receive a drill sleeve 1070.

The first screw guide 1108 can include one or more apertures 1112, 1114 configured to align a screw 170 with one or more apertures 265, 267, 269 of the stem 230. For example, the one or more apertures can include a first aperture 1112 and a second aperture 1114. The first aperture 1112 can align the screw 170 and/or the drill sleeve 1070 with the fifth aperture 269. The second aperture 1114 can align the screw 170 and/or the drill sleeve 1070 with the fourth aperture 269.

The first screw guide 1108 can include a sliding plate 1116 that can move between a first position and a second position. The sliding plate 1116 can be configured to cover the first or second aperture 1112, 1114 of the first screw guide 1108. For example, the sliding plate 1116 can include corresponding first and second apertures that align with the first and second apertures 1112, 1114 of the first screw guide 1108, respectively. When the distal arm extension 1102 is on the first side of the jig 1100, as shown in FIGS. 20A-20E, the sliding plate 1116 can be in the first position. When the sliding plate 1116 is in the first position, the sliding plate 1116 can cover the second aperture 1114 and the first aperture of the sliding plate 1116 can align with the first aperture 1112 of the first screw guide 1108 such that the first screw guide 1108 can align the screw 170 and/or the drill sleeve 1070 with the fifth aperture 269. When the distal arm extension 1102 is on the second side of the jig 1100, as shown in FIGS. 21A-21E, the sliding plate 1116 can be in the second position. When the sliding plate 1116 is in the second position, the sliding plate 1116 can cover the first aperture 1112 and the second aperture of the sliding plate 1116 can align with the second aperture 11124 of the first screw guide 1108 such that the first screw guide 1108 can align the screw 170 and/or the drill sleeve 1070 with the fourth aperture 267.

The sliding plate 1116 can be configured to move along a longitudinal axis of the first screw guide 1108 to move between the first and second positions. The sliding plate 1116 can be configured to move between the first and second positions by gravitational forces and/or the user manually moving the sliding plate 1116. In some configurations, the sliding plate 1116 can include a sliding mechanism that can move the sliding plate 1116 between the first and second positions. For example, when the distal arm extension 1102 is moved to the first side of the jig 1100, gravitational forces, the user, and/or the sliding mechanism can move the sliding plate 1116 distally relative to the body of the first screw guide 1108 to the first position such that the top aperture (e.g., the second aperture 1114) is covered and the bottom aperture (e.g., the first aperture 1112) is uncovered. As a further example, when the distal arm extension 1102 is moved to the second side of the jig 1100, gravitational forces. the user, and/or the sliding mechanism can move the sliding plate 1116 distally relative to the body of the first screw guide 1108 to the second position such that the top aperture (e.g., the first aperture 1112) is covered and the bottom aperture (e.g., the second aperture 1114) is uncovered. Advantageously, whether the surgeon is implanting the stem 230 in the left or right arm of the patient, the sliding plate 1116 can prevent the surgeon from inserting the screw 170 and/or the drill sleeve 1070 into the incorrect aperture of the first and second apertures 1112, 1116 for the procedure.

The jig 1000, 1100 may form part of a kit including a stemless bone anchor and/or a stemmed bone anchor. The stemless and/or stemmed bone anchor may include any of the features of the implants described above. The interfacing portion 1014 may be configured to engage the jig interface of the stemless bone anchor, which is the same as or similar to the stem holder interface described above, and/or the stem face of the stemmed bone anchor.

In use, the same jig 1000, 1100 may engage the jig interface of a first, stemless bone anchor or the stem face of a second, stemmed bone anchor. The stemless and/or stemmed bone anchor may include any of the features of the implants described above. For example, the jig 1000, 1100 may engage the jig interface of the stemless bone anchor and advance the stemless bone anchor into bone matter exposed at a resection of a bone. When advancing the stemless bone anchor, a force may be applied to the impaction head 1012 of the jig 1000, 1100 to apply a force perpendicular to the resection plane of the bone.

The same jig 1000, 1100 may engage the stem face of the stemmed bone anchor and advance the stemmed bone anchor to position the stem of the bone anchor in a medullary canal of the bone. When advancing the stemmed bone anchor, a force may be applied to the impaction head 1012 of the jig 1000, 1100 to apply a force aligned with a longitudinal axis of the stemmed bone anchor to embed the stem in the bone.

### B. Methods of Implanting Humeral Anchors

The humeral anchors described above can be implanted following methods discussed below in connection with FIGS. 18A-21E. These methods can advantageously employ certain tools and instruments, such as the ones described above, that can be shared among the stemless anchors 103 and the stemmed anchors 30, 230. This provides advantages in reducing the training required to complete a surgical procedure.

1. Methods of Using the Stem Holder

FIGS. 18E-18H illustrate the stem holder 900 coupled to a stem 30 and FIG. 18I illustrates the stem holder 900 coupled to the stem 30 with the stem 30 being implanted in a humerus H.

Before implanting the stem 30 into the humerus H, the surgeon can prepare the humerus H. The surgeon can resect the humerus H at the anatomic neck to separate the articular surface of the humerus H from the rest of the humerus H. The separation of the articular surface from the rest of the humerus H creates a resection surface. Optionally, the surgeon can apply a protect tool, such as a plate, to the resected surface to cover the newly exposed cancellous bone. It is important to protect the newly exposed cancellous bone because this bone is to be formed in later parts of the method to have a recess having an inner profile that matches the outer or exterior and distal surface of any of the anchors (e.g., the stemless anchor or the metaphysis portion of the stemmed anchors). The surgeon can optionally remove the protect tool and size the resected humerus H to determine which size of the stem 30, 230 (or other anchors as disclosed herein) should be used for the particular patient. Following the resection step, or the optional protection and/or sizing steps, the surgeon can ream the humerus H to form a recess or cavity in the exposed cancellous bone. The reaming step can produce a stepped internal recess or cavity in the metaphysis of the humerus H shaped to receive a humeral anchor portion, e.g., the stemless anchor 103 or a metaphysis potion of a stemmed anchor 30, 230.

After the humerus H has been prepared, the surgeon can use the stem holder 900 to employ trial anchors, which can have more easily disengaged connections with a trial head assembly or trial insert assembly than would be the case in a final implant. The trial step can enable the surgeon to choose or confirm a size to be used in the final implant. During this step, the surgeon may also use the height gauge 930 to determine an appropriate stem height for the particular patient. The surgeon may couple the stem holder 900 to the trail anchor and move the wheel 942 until the marker is 950 contacts the exposed cancellous bone. Also, the surgeon may use the graft tool 800 to create any plugs 700 from the removed humeral head.

FIGS. 18E-18H show the stem holder 900 coupled with the stem 30. The stem holder 900 can be coupled with the other stem 230 or any of the stemless anchors 103. The use of common instrumentation enables the surgeon to determine during the procedure that the stemless anchor 103 is not appropriate and then to quickly switch to the humeral stem 30, 230 following any additional preparation of the humerus H that would make the humerus H ready for the humeral stem 30, 230.

In the case of the stem 30, the stem holder 900 can grip the stem 30 in the recess thereof by engaging the tooling interfaces, e.g., the one or more apertures 51a, 51b, 53. Optionally, the surgeon can insert one or more plugs 700 into the one or more apertures 62, 64, 98 of the humeral stem 30. In the case of an elongate plug 700A, the surgeon may cut the elongate plug 700A so that the elongate plug 700A is the same length as the aperture 52, 54, 98. In the case of a plug 700B, the surgeon may insert a first plug 700B in one end of the aperture 52, 54, 98 and a second plug 700B in the other end of the aperture 52, 54, 98.

Thereafter, the distal shaft portion 32 of humeral stem 30 can be inserted through the formed recess in the resection surface and further inserted into the intramedullary canal. Once the distal shaft portion 32 is in the diaphysis of the humerus H and the metaphyseal portion 90 is in the metaphysis of the humerus H, an impaction load can be applied to the stem holder 900. In particular, an impactor, e.g., a mallet, can strike the impaction head 924 that is disposed at the proximal end 902 of the stem holder 900 driving the humeral stem 30 into firm engagement with the humerus H generally along the axis of the distal shaft portion 32 of the humeral stem 30. For example, the surgeon can apply impaction forces to the stem holder 900 until the marker 950 contacts the humerus H.

In the case of a stemless anchor 103, the stem holder 900 can grip the anchor in the recess thereof by engaging the tooling interfaces. Thereafter, the anchor 103 can be moved into the recess formed in the humerus H and pressed against the prepared surface. Thereafter, an impactor, e.g., a mallet, can be used to apply a load to the impaction head 924 at the proximal end 904 of the stem holder 900 and along the longitudinal axis thereof. The load can thus be directed transverse to, e.g., generally perpendicular to the plane of the resection surface that is formed in the resection step. Thus the inserting step can be achieved for a stemless implant 103 and for a stemmed implant such as the humeral stem 30, 230 using the same impactor instrument, e.g., the stem holder 900.

An impacting step can follow the previously described inserting step. The impacting step involves impacting an anatomic assembly 160, reverse articular body 180, and/or a spacer 150 into the stemmed anchor 30 (or another stemmed anchor 230). As discussed above, the kit 100 includes shared implant components. As such, the impacting step can be the same for the humeral stem 30, 230 as for the stemless anchors 103.

2. Methods of Using the Jig

FIGS. 19F, 20E, and 21E illustrate the jig 1000, 1100 coupled to a stem 30 or a stem 230 with the stem 30, 230 being implanted in a humerus H. Before implanting the stem 30, 230, the surgeon can prepare the humerus H with the same or similar methods as described above in relation to the method(s) of using the stem holder 900.

FIG. 19F illustrates the jig 1000 coupled with the stem 30. The jig 1000 can be coupled with the other stem 230 or any of the stemless anchors 103. The use of common instrumentation enables the surgeon to determine during the procedure that the humeral stem 30 is not appropriate and then to quickly switch to the other humeral stem 230 following any additional preparation of the humerus H that would make the humerus H ready for the humeral stem 230.

In the case of the stem 30, the jig 1000 can grip the stem 30 in the stem face by engaging the tooling interfaces, e.g., the one or more apertures 51a, 51b, 53. Optionally, the surgeon can insert one or more plugs 700 into the one or more apertures 62, 64, 98 of the humeral stem 30. In the case of an elongate plug 700A, the surgeon may cut the elongate plug 700A so that the elongate plug 700A is the same length as the aperture 52, 54, 98. In the case of a plug 700B, the surgeon may insert a first plug 700B in one end of the aperture 52, 54, 98 and a second plug 700B in the other end of the aperture 52, 54, 98.

Thereafter, the distal shaft portion 32 of humeral stem 30 can be inserted through the formed recess in the resection surface and further inserted into the intramedullary canal. Once the distal shaft portion 32 is in the diaphysis of the humerus H and the metaphyseal portion 90 is in the metaphysis of the humerus H, an impaction load can be applied to the jig 1000. In particular, an impactor, e.g., a mallet, can strike the impaction head 1012 that is disposed at the proximal end 1002 of the jig 1000 driving the humeral stem 30 into firm engagement with the humerus H generally along the axis of the distal shaft portion 32 of the humeral stem 30. For example, the surgeon can apply impaction forces to the jig 1000 until the marker 1053 contacts the humerus H.

An impacting step can follow the previously described inserting step. The impacting step involves impacting an anatomic assembly 160, reverse articular body 180, and/or a spacer 150 into the stemmed anchor 30 (or another stemmed anchor 230). As discussed above, the kit 100 includes shared implant components. As such, the impacting step can be the same for the humeral stem 30, 230 as for the stemless anchors 103.

After the impacting step, in the case of the stem 30, a securing step can be performed. A minimal skin incision may be performed at a planned entry point of the screw 170. The drill sleeve 1070 can be inserted into one of the screw guides 1064. The drill sleeve 1070 can be advanced through the incised entry point to the humerus H. A tool can be inserted through the channel 1072 of the drill sleeve 1070 to create a hole in the humerus H and through one of the apertures 62, 64. For example, a drill can be used to create the hole. The same or another tool can be sued to insert a screw 170 into the channel 1072 of the drill sleeve 1070 and into the previously created hole. These steps can be repeated to insert a screw 170 into the other one of the apertures 62, 64.

FIGS. 20E and 21E illustrate the jig 1100 coupled with the stem 230. The jig 1100 can be coupled with the other stem 30 or any of the stemless anchors 103. In the case of the stem 230, the jig 1100 can grip the stem 230 in the stem face 250 by engaging the tooling interfaces, e.g., the one or more apertures 251a, 251b, 253. Optionally, one or more plugs 700 can be inserted into the one or more apertures 262, 264, 265, 267, 269, 298 of the humeral stem 230. For example, the one or more plugs 700 can be inserted into the fourth aperture 267 of the stem 230 when implanting the stem 230 in the left arm of the patient (FIG. 20E). Alternatively, the one or more plugs 700 can be inserted into the fifth aperture 269 of the stem 230 when implanting the stem 230 in the right arm of the patient (FIG. 21E). In the case of an elongate plug 700A, the surgeon may cut the elongate plug 700A so that the elongate plug 700A is the same length as the aperture 267, 269. In the case of a plug 700B, the surgeon may insert a first plug 700B in one end of the aperture 267, 269 and a second plug 700B in the other end of the aperture 267, 269.

Thereafter, the distal arm extension 1102 can be moved to the first side of the jig 1100 (FIG. 20E) or the second side of the jig 1100 (FIG. 21E) depending on which arm of the patient is being operated on. The distal arm extension 1102 can be secured in this position by tightening the distal-most fastening screw 1062. The distal shaft 232 of humeral stem 230 can be inserted through the formed recess in the resection surface and further inserted into the intramedullary canal. Once the distal shaft 232 is in the diaphysis of the humerus H and the metaphyseal portion 290 is in the metaphysis of the humerus H, an impaction load can be applied to the jig 1100. In particular, an impactor, e.g., a mallet, can strike the impaction head 1012 that is disposed at the proximal end 1002 of the jig 1100 driving the humeral stem 230 into firm engagement with the humerus H generally along the axis of the distal shaft 232 of the humeral stem 230. For example, the surgeon can apply impaction forces to the jig 1100 until the marker 1053 contacts the humerus H.

An impacting step can follow the previously described inserting step. The impacting step involves impacting an anatomic assembly 160, reverse articular body 180, and/or a spacer 150 into the stemmed anchor 230 (or another stemmed anchor 30). As discussed above, the kit 100 includes shared implant components. As such, the impacting step can be the same for the humeral stem 30, 230 as for the stemless anchors 103.

After the impacting step, in the case of the stem 230, a securing step can be performed. A minimal skin incision may be performed at a planned entry point of the screw 170. The drill sleeve 1070 can be inserted into one of the screw guides 1064, 1108, 1110. The drill sleeve 1070 can be advanced through the incised entry point to the humerus H. A tool can be inserted through the channel 1072 of the drill sleeve 1070 to create a hole in the humerus H and through one of the apertures 262, 264, 265, 267, 269. For example, a drill can be used to create the hole. The same or another tool can be used to insert a screw 170 into the channel 1072 of the drill sleeve 1070 and into the previously created hole. These steps can be repeated to insert a screw 170 into the other ones of the apertures 262, 264, 265, 267, 269, as needed.

### Other Variations and Terminology

Although certain embodiments have been described herein, the implants and methods described herein can interchangeably use any articular component, as the context may dictate.

As used herein, the relative terms “proximal” and “distal” shall be defined from the perspective of the implant. Thus, proximal refers to the direction of the articular component and distal refers to the direction of an anchor component, such as a stem of a humeral anchor or a thread or porous surface or other anchoring structure of a stemless anchor when the implant is assembled.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1” includes “1.” Phrases preceded by a term such as “substantially,” “generally,” and the like include the recited phrase and should be interpreted based on the circumstances (e.g., as much as reasonably possible under the circumstances). For example, “substantially spherical” includes “spherical.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Although certain embodiments and examples have been described herein, it should be emphasized that many variations and modifications may be made to the humeral head assembly shown and described in the present disclosure, the elements of which are to be understood as being differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations are to be interpreted broadly based on the language employed and not limited to the examples described in the present specification or during the prosecution of the application, which examples are to be construed as non-exclusive.

Any methods disclosed herein need not be performed in the order recited. The methods disclosed herein include certain actions taken by a practitioner; however, they can also include any third-party instruction of those actions, either expressly or by implication. For example, actions such as “coupling a glenoid guide with the glenoid rim” include “instructing coupling of a glenoid guide with a glenoid rim.”

1. A stem for a shoulder prosthesis comprising:
a medial side;
a lateral side opposite the medial side; and
a plurality of apertures, each aperture of the plurality of apertures configured to receive a screw or one or more plugs, the plurality of apertures comprising a first aperture and a second aperture, wherein the first aperture is positioned proximal to the second aperture, wherein each of the first and second apertures comprises a first opening on the medial side, a second opening on the lateral side, and a length measured along a longitudinal centerline therebetween, the longitudinal centerline of at least one of the first and second apertures being angled relative to a longitudinal plane extending in a medial-lateral direction of the stem.

2. The stem of claim 1, wherein each of the first and second apertures is angled in an anterior-posterior direction relative to the longitudinal plane.

3. The stem of claim 1, wherein the first and second apertures are angled in opposite directions relative to the longitudinal plane.

4. The stem of claim 1, further comprising:
a distal shaft portion adapted to be anchored in a medullary canal of a humerus,
a proximal portion having a stem face, and
a metaphyseal portion extending between and connecting the distal shaft portion and the proximal portion.

5. The stem of claim 4, wherein the metaphyseal portion comprises a medial portion and first and second lateral arms.

6. The stem of claim 4, wherein the distal shaft portion comprises the plurality of apertures.

7. The stem of claim 4, wherein the distal shaft portion comprises a plurality of grooves extending in a longitudinal direction, wherein the plurality of grooves are circumferentially spaced apart.

8. The stem of claim 7, wherein each of the plurality of grooves narrows toward a distal tip of the stem.

9. The stem of claim 7, wherein the first aperture is positioned proximal to the plurality of grooves.

10. The stem of claim 7, wherein the second aperture extends through at least one of the plurality of grooves.

11. The stem of claim 1, wherein the plurality of apertures further comprises a third aperture positioned distal to the second aperture.

12. The stem of claim 1, wherein the longitudinal centerline of each of the first and second apertures is angled 30° relative to the longitudinal plane of the stem.

13. A system, comprising:
the stem of claim 1, wherein each aperture of the plurality of apertures comprises a first opening, a second opening, and a length measured along a longitudinal centerline therebetween; and
at least one plug configured to be received by one or more apertures of the plurality of apertures of the stem.

14. The system of claim 13, wherein the at least one plug comprises at least one elongate plug, wherein a width of the at least one elongate plug is less than a length thereof.

15. The stem of claim 14, wherein the length of the one or more apertures of the plurality of apertures is less than the length of the at least one elongate plug.

16. The system of claim 14, wherein the one or more apertures of the plurality of apertures are configured to receive the at least one elongate plug along the entire length of the one or more apertures.

17. The system of claim 13, wherein a width of the at least one plug is greater than a length thereof, wherein the length of the one or more apertures of the plurality of apertures is greater than the length of the at least one plug.

18. The system of claim 17, wherein two or more of the plugs are configured to be inserted into an aperture of the one or more apertures along the longitudinal centerline.

19. A kit comprising:
a stem for a shoulder prosthesis comprising:
a medial side;
a lateral side opposite the medial side; and
a plurality of apertures, each aperture of the plurality of apertures configured to receive a screw or one or more plugs, the plurality of apertures comprising a first aperture and a second aperture, wherein the first aperture is positioned proximal to the second aperture, wherein each of the first and second apertures comprises a first opening on the medial side, a second opening on the lateral side, and a length measured along a longitudinal centerline therebetween, the longitudinal centerline of at least one of the first and second apertures being angled relative to a longitudinal plane extending in a medial-lateral direction of the stem;
a reverse insert, the reverse insert having a proximal portion and a distal portion, the proximal portion of the reverse insert including a concave surface configured to receive a glenosphere and the distal portion comprising a protrusion, wherein the reverse insert is configured to directly couple to the stem;
an anatomical articular component having a proximal portion including a convex surface and a distal portion including a protrusion, wherein the anatomical articular component is configured to directly couple to the stem; and
a spacer comprising a proximal portion and a distal portion, the spacer configured to couple the reverse insert or the anatomical articular component to the stem, wherein the proximal portion of the spacer is symmetric.

20. The kit of claim 19, wherein each of the first and second apertures is angled in an anterior-posterior direction relative to the longitudinal plane.

21.-86. (canceled)
